(19) United States
(12) Patent Application Publication
(10) Pub. No.: US 2026/0217861 A1
(43) Pub. Date: Jul. 30, 2026

(54) ANTIBODIES FOR USE AS COAGULANTS

(71) Applicant: JJULIUS-MAXIMILIANS-UNIVERSITÄT-WÜRZBURG, Würzburg (DE)

(72) Inventors: Bernhardt NIESWANDT, Eibelstadt (DE); Sarah BECK, Wuerzburg (DE); David STEGNER, Wuerzburg (DE)

(21) Appl. No.: 19/142,118

(22) PCT Filed: Dec. 22, 2023
(86) PCT No.: PCT/EP2023/087547

§ 371 Date: Jun. 20, 2025

(30) Foreign Application Priority Data
Dec. 22, 2022 (EP) 22216237.2

(51) Int. Cl. C07K 16/36; A61K 35/16; A61K 35/19; A61K 39/395; A61P 7/04

(52) U.S. Cl. CPC C07K 16/36; A61K 35/16; A61K 35/19; A61K 39/3955; A61P 7/04

(57) ABSTRACT

The present invention provides novel antibodies, fragments or derivatives thereof—as well as respective (medical) uses of those compounds, such as their use as a coagulant, and/or their use in the treatment or prevention of a haemorrhagic condition.

### BACKGROUND

Platelet activation and subsequent thrombus formation at sites of vascular injury is crucial for normal haemostasis, but it can also cause myocardial infarction and stroke. Platelet adhesion and activation is a multistep process involving multiple platelet receptor-ligand interactions. Upon vessel wall injury, circulating platelets are rapidly decelerated by transient interactions of the glycoprotein (GP) Ib-V-IX complex with von Willebrand factor (vWF) immobilized on the exposed subendothelial extracellular matrix (e.g. on collagen).

In general, haemostasis is the physiological mechanism that limits bleeding after blood vessel injury through intertwined activations of circulating platelets and the plasmatic coagulation cascade¹. The adhesion of platelets to extracellular matrix proteins and von Willebrand factor (VWF) initiates the haemostatic response that is supported by exposure of subendothelial tissue factor (TF), which triggers coagulation and local thrombin generation². This results in a fibrin network encasing platelets in a stable thrombus³. Thrombin generation requires feed-forward reactions that involve platelet activation by thrombin-mediated cleavage and activation of G-protein coupled protease-activated receptors (PARs)⁴ and amplification of coagulation reactions on the surface of activated platelets⁵. Generated thrombin forms fibrin and thereby stabilises thrombi through platelet receptor GPIIb/IIIa engagement and activates FXIII to crosslink fibrin fibres⁶. These processes are regulated with high fidelity⁷ to ensure efficient haemostasis while preventing thrombosis and thrombo-inflammatory diseases^{8,9}. In addition, thrombin activity in the circulation is limited by specific plasmatic coagulation inhibitors and by thrombomodulin on endothelial cells, which captures thrombin to initiate the coagulation regulatory and vascular protective protein C pathway¹⁰.

The glycoprotein (GP) Ib-IX complex mediates platelet binding to VWF and is crucial for haemostasis. Mutations in GP1BA, GP1BB or GP9 cause the Bernard-Soulier syndrome (BSS), a rare bleeding disorder characterised by giant platelets^{11,12}. GPV is associated with the GPIb-IX complex, but not required for GPIb expression or functional interactions¹³. GPV is an abundant 88 kDa platelet/megakaryocyte-specific leucine-rich repeat (LRR) transmembrane protein¹⁴ that interacts with collagen¹⁵ and has minor importance for platelet function^{16,17}. GPV is proteolytically cleaved by thrombin during thrombus formation^{18,19}, but the physiological roles of the shed 69 kDa extracellular fragment in haemostasis and thrombosis have remained elusive.

Prevention of thrombosis while preserving haemostasis has been a central goal of antithrombotic drug development. Despite the broader application and safety of target selective oral anticoagulants, preventing bleeding complications remains an unmet clinical need. Although a recent study provides proof of principle that platelet mediated thrombin generation can rescue bleeding defects due to increased fibrinolysis⁴⁷, there is an unmet clinical need for more general and specific spatio-temporal control of fibrin formation. Novel haemostatic agents are approved or in development to bypass genetic or acquired deficiencies in the coagulation cascade⁴⁸, but platelet transfusion remains the only therapeutic option to acutely restore defective platelet function or severe forms of thrombocytopenia to secure haemostasis.

WO 2017/109180 A1 describes GPV inhibitors for use as coagulants. The examples mention a monoclonal rat anti-mouse GPV antibody. However, the investigated antibodies are described as having no influence on thrombin-mediated cleavage of GPV.

The inventors of the present application surprisingly found that antibodies that inhibit thrombin-mediated cleavage of GPV have excellent pro-coagulatory activity and can be used to promote haemostasis.

### SUMMARY OF THE INVENTION

The present invention particularly relates to the subject-matter as defined in the claims. It e.g. provides novel antibodies, fragments or derivatives thereof as well as respective medical uses.

The present invention specifically relates to the following items [1] to [54]:

[1] An antibody or a functional fragment or derivative thereof capable of binding to human platelet glycoprotein V (GPV), wherein said antibody, functional fragment or derivative inhibits thrombin-mediated cleavage of GPV.

[2] The antibody, fragment or derivative according to [1], wherein said antibody, fragment or derivative is capable of binding to the extracellular domain of GPV.

[3] The antibody, fragment or derivative according to [2], wherein said antibody, fragment or derivative is capable of binding to a region of the extracellular domain of GPV which is different from the collagen-binding site of GPV.

[4] An antibody or a functional fragment or a functional derivative thereof according to any one of [1] to [3], wherein said antibody, fragment or derivative does not delay collagen-induced aggregation.

[5] The antibody or fragment or derivative thereof according to any one of [1] to [4], wherein said antibody, fragment or derivative accelerates fibrin formation, increases fibrin formation and/or improves formed fibrin structure, in particular accelerates and increases fibrin formation, particularly local fibrin formation as opposed to systemic fibrin formation.

[6] The antibody or fragment or derivative thereof according to any one of [1] to [5], wherein said antibody, fragment or derivative does not affect the number of platelets in a subject upon administration to the subject.

[7] An antibody or a fragment or derivative thereof, preferably according to any one of [1] to [6], comprising (i) a V_{H} domain comprising a CDR1 having an amino acid sequence as shown in SEQ ID NO:15, a CDR2 having an amino acid sequence as shown in SEQ ID NO:16, and a CDR3 having an amino acid sequence as shown in SEQ ID NO:17, and (ii) a V_{L} domain comprising a CDR1 having an amino acid sequence in accordance with the amino acid sequence as shown in SEQ ID NO:18, a CDR2 having an amino acid sequence in accordance with the amino acid sequence as shown in SEQ ID NO:19, and a CDR3 having an amino acid sequence in accordance with the amino acid sequence as shown in SEQ ID NO:20, or
wherein the antibody competes for binding to GPV with an antibody comprising (i) a V_{H} domain comprising a CDR1 having an amino acid sequence as shown in SEQ ID NO:15, a CDR2 having an amino acid sequence as shown in SEQ ID NO:16, and a CDR3 having an amino acid sequence as shown in SEQ ID NO:17, and (ii) a V_{L} domain comprising a CDR1 having an amino acid sequence in accordance with the amino acid sequence as shown in SEQ ID NO:18, a CDR2 having an amino acid sequence in accordance with the amino acid sequence as shown in SEQ ID NO:19, and a CDR3 having an amino acid sequence in accordance with the amino acid sequence as shown in SEQ ID NO:20.

[8] The antibody, fragment or derivative according to any one of [1] to [7], wherein the V_{H} domain comprises an amino acid sequence as shown in SEQ ID NO:21, and the V_{L} domain comprises an amino acid sequence as shown in SEQ ID NO:23, or wherein the antibody, fragment or derivative specifically competes for binding to a GPV epitope bound by an antibody with the V_{H} domain comprising an amino acid sequence as shown in SEQ ID NO:21, and the V_{L} domain comprising an amino acid sequence as shown in SEQ ID NO:23.

[9] An antibody or a fragment or derivative thereof, preferably according to any one of [1] to [6], comprising (i) a V_{H} domain comprising a CDR1 having an amino acid sequence as shown in SEQ ID NO:1, a CDR2 having an amino acid sequence as shown in SEQ ID NO:2, and a CDR3 having an amino acid sequence as shown in SEQ ID NO:3, and (ii) a V_{L} domain comprising a CDR1 having an amino acid sequence in accordance with the amino acid sequence as shown in SEQ ID NO:4, a CDR2 having an amino acid sequence in accordance with the amino acid sequence as shown in SEQ ID NO:5, and a CDR3 having an amino acid sequence in accordance with the amino acid sequence as shown in SEQ ID NO:6, or
wherein the antibody competes for binding to GPV with an antibody comprising (i) a V_{H} domain comprising a CDR1 having an amino acid sequence as shown in SEQ ID NO:1, a CDR2 having an amino acid sequence as shown in SEQ ID NO:2, and a CDR3 having an amino acid sequence as shown in SEQ ID NO:3, and (ii) a V_{L} domain comprising a CDR1 having an amino acid sequence in accordance with the amino acid sequence as shown in SEQ ID NO:4, a CDR2 having an amino acid sequence in accordance with the amino acid sequence as shown in SEQ ID NO:5, and a CDR3 having an amino acid sequence in accordance with the amino acid sequence as shown in SEQ ID NO:6.

[10] The antibody, fragment or derivative according to [1] to [6] or [9], wherein the V_{H} domain comprises an amino acid sequence as shown in SEQ ID NO:7, and the V_{L} domain comprises an amino acid sequence as shown in SEQ ID NO:9 or wherein the antibody, fragment or derivative specifically competes for binding to a GPV epitope bound by an antibody with the V_{H} domain comprising an amino acid sequence as shown in SEQ ID NO:7, and the V_{L} domain comprising an amino acid sequence as shown in SEQ ID NO:9.

[11] The antibody, fragment or derivative according to any one of [1] to [10], wherein said antibody is a monoclonal antibody.

[12]A nucleic acid encoding an antibody, fragment or derivative according to any one of [1] to [11].

[13]A host cell comprising the nucleic acid of [12].

[14]A method of preparing the antibody, fragment or derivative according to any one of [1] to [11], comprising culturing the host cell of [13] under suitable conditions allowing expression of the antibody, fragment or derivative, and recovering the antibody, fragment or derivative.

[15]A pharmaceutical composition comprising the antibody, fragment or derivative according to any one of [1] to [11] or the nucleic acid according to [12].

[16] The pharmaceutical composition of [15], wherein the composition further comprises a pharmaceutically acceptable excipient.

[17] The antibody, fragment, or derivative according to any one of [1] to [11] for use in medicine, particularly for use in improving, preferably restoring, haemostasis.

[18] The antibody, fragment, or derivative according to any one of [1] to [11] for use in the treatment or prevention of a haemorrhagic condition and/or for use in reversing the effect of anti-platelet and/or anti-coagulant medication, for example reversing the effect in emergency bleeding control.

[19] The antibody, fragment, or derivative for use according to [17] or [18], wherein i) said use in medicine or haemorrhagic condition is due to/caused by a platelet disorder, especially wherein said platelet disorder is characterized by a decreased number of platelets and/or ii) said use in medicine is due to a condition selected from or said haemorrhagic condition is selected from the group consisting of inflammatory bleeding, haemophilia/FVIII, bleeding due to anti-platelet therapy, bleeding due to anti-coagulant therapy, haemorrhagic stroke, excessive bleeding due to sepsis, excessive bleeding due to thrombocytopenia, excessive bleeding due to disseminated intravascular coagulation (DIC), excessive bleeding due to chemotherapy, excessive bleeding due to haemolytic-uremic syndrome, excessive bleeding upon administration of soluble GPV, and excessive bleeding due to HIV infection.

[20] The antibody, fragment, or derivative for use according to [17] or [18], wherein said use in medicine or haemorrhagic condition is due to/caused by a platelet disorder, especially wherein said platelet disorder is characterized by a decreased number of platelets, and/or wherein said use in medicine or haemorrhagic condition is anti-hemorrhagic therapy, in particular for increasing and/or normalizing bleeding times in patients having a prolonged bleeding time, preferably a prolonged bleeding time resulting from thrombocytopenia, genetic defects, and/or anti-platelet therapy (such as clopidogrel).

[21] The antibody, fragment, or derivative for use according to [17] or [18], wherein said use in medicine is due to a condition selected from or said haemorrhagic condition is selected from the group consisting of inflammatory bleeding, haemophilia/FVIII, bleeding due to anti-platelet therapy, bleeding due to anti-coagulant therapy, haemorrhagic stroke, excessive bleeding due to sepsis, excessive bleeding due to thrombocytopenia, excessive bleeding due to disseminated intravascular coagulation (DIC), excessive bleeding due to chemotherapy, excessive bleeding due to haemolytic-uremic syndrome, excessive bleeding upon administration of soluble GPV, and excessive bleeding due to HIV infection.

[22] The antibody, fragment, or derivative for use according to any one of [17] to [21], wherein said antibody, fragment, or derivative i) reduces cleavage of GPV by thrombin, and/or ii) accelerates fibrin formation, and/or iii) does not affect the number of platelets in a subject upon administration to the subject, iv) is used as a coagulant.

[23] The antibody, fragment, or derivative for use according to any one of [17] to [22], wherein said antibody, fragment, or derivative reduces cleavage of GPV by thrombin.

[24] The antibody, fragment, or derivative for use according to any one of [17] to [23], wherein said antibody, fragment, or derivative accelerates fibrin formation and/or improves fibrin structure.

[25] The antibody, fragment, or derivative for use according to any one of [17] to [24], wherein said antibody, fragment, or derivative does not affect the number of platelets in a subject upon administration to the subject.

[26] The antibody, fragment, or derivative for use according to any one of [17] to [25], wherein said antibody, fragment, or derivative is used as a coagulant.

[27] The nucleic acid according to [12] for use in medicine, particularly for use in improving haemostasis.

[28] The nucleic acid according to [12] for use in the treatment or prevention of a haemorrhagic condition.

[29] The nucleic acid for use according to [28], wherein i) said haemorrhagic condition is caused by a platelet disorder, especially wherein said platelet disorder is characterized by a decreased number of platelets and/or ii) said haemorrhagic condition is selected from the group consisting of inflammatory bleeding, haemophilia/FVIII, bleeding due to anti-platelet therapy, bleeding due to anti-coagulant therapy, haemorrhagic stroke, excessive bleeding due to sepsis, excessive bleeding due to thrombocytopenia, excessive bleeding due to disseminated intravascular coagulation (DIC), excessive bleeding due to chemotherapy, excessive bleeding due to haemolytic-uremic syndrome, excessive bleeding upon administration of soluble GPV, and excessive bleeding due to HIV infection.

[30] The nucleic acid for use according to [28] or [29], wherein said haemorrhagic condition is caused by a platelet disorder, especially wherein said platelet disorder is characterized by a decreased number of platelets.

[31] The nucleic acid for use according to [28], [29] or [30], wherein said haemorrhagic condition is selected from the group consisting of inflammatory bleeding, haemophilia/FVIII, bleeding due to anti-platelet therapy, bleeding due to anti-coagulant therapy, haemorrhagic stroke, excessive bleeding due to sepsis, excessive bleeding due to thrombocytopenia, excessive bleeding due to disseminated intravascular coagulation (DIC), excessive bleeding due to chemotherapy, excessive bleeding due to haemolytic-uremic syndrome, excessive bleeding upon administration of soluble GPV, and excessive bleeding due to HIV infection.

[32] The nucleic acid for use according to any one of [27] to [31], wherein said nucleic acid i) reduces cleavage of GPV by thrombin, and/or ii) accelerates fibrin formation, and/or iii) does not affect the number of platelets in a subject upon administration to the subject, iv) is used as a coagulant.

[33] The nucleic acid for use according to any one of [27] to [31], wherein said nucleic acid reduces cleavage of GPV by thrombin.

[34] The nucleic acid for use according to any one of [27] to [31], wherein said nucleic acid accelerates fibrin formation.

[35] The nucleic acid for use according to any one of [27] to [31], wherein said nucleic acid does not affect the number of platelets in a subject upon administration to the subject.

[36] The nucleic acid for use according to any one of [27] to [31], wherein said nucleic acid is used as a coagulant.

[37] The pharmaceutical composition according to [15] or [16] for use in medicine, particularly for use in improving haemostasis.

[38] The pharmaceutical composition according to [15] or [16] for use in the treatment or prevention of a haemorrhagic condition.

[39] The pharmaceutical composition for use according to [37] or [38], wherein said haemorrhagic condition is selected from the group consisting of inflammatory bleeding, haemophilia/FVIII, bleeding due to anti-platelet therapy, bleeding due to anti-coagulant therapy, haemorrhagic stroke, excessive bleeding due to sepsis, excessive bleeding due to thrombocytopenia, excessive bleeding due to disseminated intravascular coagulation (DIC), excessive bleeding due to chemotherapy, excessive bleeding due to haemolytic-uremic syndrome, excessive bleeding upon administration of soluble GPV, and excessive bleeding due to HIV infection.

[40] The pharmaceutical composition for use according to [37] or [38], wherein said haemorrhagic condition is caused by a platelet disorder, especially wherein said platelet disorder is characterized by a decreased number of platelets.

[41] The pharmaceutical composition for use according to [37] or [38], wherein said haemorrhagic condition is selected from the group consisting of inflammatory bleeding, haemophilia/FVIII, bleeding due to anti-platelet therapy, bleeding due to anti-coagulant therapy, haemorrhagic stroke, excessive bleeding due to sepsis, excessive bleeding due to thrombocytopenia, excessive bleeding due to disseminated intravascular coagulation (DIC), excessive bleeding due to chemotherapy, excessive bleeding due to haemolytic-uremic syndrome, excessive bleeding upon administration of soluble GPV, and excessive bleeding due to HIV infection.

[42] The pharmaceutical composition for use according to any one of [37] to [41], wherein said pharmaceutical composition i) reduces cleavage of GPV by thrombin, and/or ii) accelerates fibrin formation, and/or iii) does not affect the number of platelets in a subject upon administration to the subject, iv) is used as a coagulant.

[43] The pharmaceutical composition for use according to any one of [37] to [41], wherein said pharmaceutical composition reduces cleavage of GPV by thrombin.

[44] The pharmaceutical composition for use according to any one of [37] to [41], wherein said pharmaceutical composition accelerates fibrin formation.

[45] The pharmaceutical composition for use according to any one of [37] to [41], wherein said pharmaceutical composition does not affect the number of platelets in a subject upon administration to the subject.

[46] The pharmaceutical composition for use according to any one of [37] to [41], wherein said pharmaceutical composition is used as a coagulant.

[47] The antibody, fragment, derivative, nucleic acid, or pharmaceutical composition for use according to any one of [17] to [46], wherein said treatment or prevention comprises administering to a subject, preferably to a human subject, a pharmaceutically effective amount of said antibody, fragment, derivative, nucleic acid, or pharmaceutical composition.

[48] The antibody, fragment, derivative, nucleic acid, or pharmaceutical composition for use according to any one of [17] to [47], wherein said treatment or prevention further comprises administering to said subject a coagulant other than said inhibitor.

[49] The antibody, fragment, derivative, nucleic acid, or pharmaceutical composition for use according to [48], wherein said coagulant other than said inhibitor is selected from the group consisting of an anti-fibrinolytic agent, a platelet concentrate, a coagulation factor concentrate and fresh frozen plasma.

[50]A host cell comprising an antibody or fragment or derivative thereof according to any one of [1] to [11] or a nucleic acid according to [12].

[51]A method for producing an antibody or fragment or derivative thereof according to any one of [1] to [11] comprising using a host cell according to [50].

[52]A method of treating a haemorrhagic condition in a subject, preferably a human, comprising administering to the subject an effective amount of an antibody or fragment or derivative thereof according to any one of [1] to [11], a nucleic acid according to [12], or a pharmaceutical composition according to [15] or [16].

[53] The method of [52], further characterized as described in any one of [17] to [49].

[54] An antibody or a fragment or derivative thereof, binding to the same epitope as the antibody as defined in [8], optionally wherein said antibody, fragment or derivative is an antibody, fragment or derivative in accordance with any one of [1] to [11].

The anti-platelet therapy of this disclosure (as also mentioned above) can involve, but is not limited to, aspirin, ADP receptor inhibitors (such as Clopidogrel, Prasugrel or Ticagrelor), anti-GPVI treatment (e.g. Glencozimab), spleen tyrosine kinase inhibitors (e.g. Fostamatinib), Bruton's tyrosin kinase inhibitors, GPIbα inhibitors (e.g. Volociximab), dipyridamole or protease-activated receptor-1 inhibitors (e.g. Vorapaxar). Conditions of anticoagulation that may be treated as described in this disclosure (as also mentioned above) may be caused by previous intake of warfarin, heparin, low molecular weight heparin (LMWH, such as enoxaparin, dalteparin or tinzaparin), activators of antithrombin III (such as fondaparinux), thrombin inhibitors (e.g. dabigatran) or inhibitors of factor Xa (such as Rivaroxaban, Edoxaban or Apixaban).

### DESCRIPTION OF THE DRAWINGS

FIG. 1: Platelet thrombin hyperresponsiveness and accelerated thrombus formation in GPV mutant mice. (A) Quantification and (B) representative images of thrombus formation upon FeCl₃-induced injury of mesenteric arterioles in Gp5^{-/-} or WT mice. Thrombus formation in no more than two arterioles of each mouse were analysed; data points represent measurements of one arteriole. WT: n=18 arterioles, Gp5^{-/-} n=12 arterioles, two-tailed unpaired t-test, p<0.0001. (C) Quantification of thrombus formation and (D) representative images upon FeCl₃-induced injury of mesenteric arterioles in Gp5^{dThr} or WT mice. At most two arterioles of each mouse were analysed. Each dot represents one arteriole. #: indicates occlusive thrombus formation. WT: n=14, Gp5^{dThr} n=18, two-tailed unpaired t-test, p=0.0010. (E) Increased P-selectin exposure of GPV-deficient platelets at threshold thrombin concentrations. Mean±SD, n=4, two-tailed unpaired t-test with Welch's correction. (F) Flow cytometry reveals unaltered reactivity of Gp5^{dThr} platelets upon thrombin stimulation compared to WT controls. Mean±SD, n=4, two-tailed unpaired t-test with Welch's correction. (G, H) Washed platelets were stimulated with the indicated agonists and light transmission of washed platelets upon stimulation with the indicated PAR4 peptide (G) or thrombin (H) concentrations was recorded on a Apact four-channel aggregometer over 10 min. Representative curves for n=3-4 and maximum aggregation expressed as mean±SD. One way ANOVA followed by Tukey's multiple comparisons test. Strain matched controls were used. *P<0.05; **P<0.01; ***P<0.001.

FIG. 2: GPV alters fibrin formation and localises to fibrin fibres outside the thrombus after thrombin cleavage. (A) Recalcified whole blood was perfused over collagen/tissue factor (TF)-coated microspots for 6 min at a wall shear rate of 1000 s⁻¹. Time-dependent fibrin generation of Gp5^{-/-} and WT mice was quantified. mean±SEM, n≥3. Two-tailed unpaired t-test with Welch's correction. (B) Representative microscopic images of platelet thrombus formation (anti-GPIX AF647) and fibrin formation (fibrin(ogen) AF488) on collagen/TF spots after 6 min of flow. Scale bar: 20 μm. (C) Quantification of time to fibrin formation. One dot represents one animal. n=4, Mann-Whitney test. (D-F) Fibrin formation on collagen/TF microspots of Gp5^{dThr} and WT mice. Quantification of fibrin formation (F) and time to fibrin formation (F) and representative images (E). Staining is same as in (B). (D) Values are depicted as mean±SEM, WT: n=6, Gp5^{dThr}: n=8, Two-tailed unpaired t-test with Welch's correction. (F) WT: n=5, Gp5^{dThr}: n=4, Mann-Whitney test. SAC: surface area coverage. (G) Subtraction heatmap of parameters of thrombus (indicated by increased multilayer and contraction score) and fibrin formation in mutant mice compared to their WT controls. Mean averages were normalised to the respective WT control. (H) Pulldown of sGPV from WT platelets using streptavidin beads after stimulation with biotinylated thrombin in the presence and absence of the matrix metalloproteinase inhibitor GM6001. No pulldown of sGPV from Gp5^{dThr} platelets nor in the presence of hirudin, which prevents GPV cleavage by thrombin. Eluates were analysed by Western blotting using GPV-specific antibodies and streptavidin-HRP. Representative result of 5 independent experiments. M: marker, mGPV: murine GPV, Arrow indicates pulldown of sGPV (thrombin cleaved GPV). (I, J) Recalcified blood was perfused over collagen/TF microspots. Samples were stained for platelets (anti-GPIX), fibrin(ogen) and GPV, fixed and analysed with a Zeiss Airyscan microscope using super-resolution mode. (I) Representative WT image and Zoom in. Scale bar: 4 μm. (J) Quantification of GPV intensities inside fibrin-rich, but non-platelet area. Background GPV intensity in Gp5^{-/-} images is displayed as dashed line. 2way ANOVA followed by Tukey's multiple comparisons test, n=12 ROI per group representing n=4 mice. For detailed analysis see FIG. 11. *P<0.05; **P<0.01; ***P<0.001.

FIG. 3: rhGPV reduces fibrin formation and thereby protects from occlusive thrombosis and ischaemic stroke. (A) Simplified scheme of full-length and recombinant ectodomain of human GPV. rhGPV contains the thrombin and ADAM cleavage sites and a C-terminal His-tag. (B) Maximum projection of static fibrin polymerization induced by thrombin (upper panel) or batroxobin (lower panel) in the absence or presence of rhGPV. Fluorophore labelled fibrin(ogen)(1^{st} and 3^{rd} panel), staining for hGPV (2^{nd} and 4^{th} panel as well as zoom-in). Scale bar: 20 μm. (C) Quantification of thrombus formation after mechanical injury of the abdominal aorta. WT: n=18, WT+rhGPV: n=15, Fisher's exact test. (D) Representative images and (E) quantification of thrombus formation upon FeCl₃-induced injury of mesenteric arterioles of rhGPV-treated and WT mice. #: indicates occlusive thrombus formation. WT: n=15, WT+rhGPV: n=15, Fisher's exact test. (C, E) Data points represent measurements of one vessel. Thrombus formation in no more than two arterioles of each mouse were analysed. (F) Quantification of infarct volumes in tMCAO model of ischaemic stroke. WT: n=13, WT+rhGPV: n=6, Gp5^{-/-}: n=6, Gp5^{dThr}: n=7, two-tailed Kruskal-Wallis test with Dunn's post-test. Of note, rhGPV-treatment neither triggered large intracranial haemorrhages, nor elevated mortality (WT: 2/15; WT+rhGPV: 1/7; Gp5^{-/-}: 0/6, Gp5^{dThr}: 1/8). (G) Three consecutive TTC-stained brain sections of one representative mouse. Withish tissue: infarct (H) Neuroscore displaying behavioural outcome after tMCAO. (I) Unaltered tail bleeding time after rhGPV-treatment. WT: n=9, WT+rhGPV: n=10. Ctrl: Human donor, rhGPV: 20 μg/mouse injected prior to surgery. *P<0.05;**P<0.01;***P<0.001.

FIG. 4: The anti-mGPV antibody DOM/B interferes with thrombin-mediated cleavage of GPV and reproduces the Gp5^{-/-} phenotype. (A) Platelets were incubated with the indicated antibody (10 μg/ml) or non-immune rat IgG (depicted as WT) prior to thrombin stimulation. Thrombin-mediated cleavage of GPV was assessed by flow cytometry. Mean±SD, n=3. (B-E) Recalcified whole blood was incubated in vitro with 10 μg/ml anti-mGPV antibody DOM/B or DOM/C prior to perfusion over collagen/TF spots. Quantification of fibrin generation during blood flow (B, E) and time to fibrin formation (C, E). (B) n=7, Mann-Whitney test. (C) n=8, two-tailed unpaired t-test. (D) n=3, Mann-Whitney test. (E) WT: n=6, WT+DOM/C: n=5 Mann-Whitney test. SAC: Surface area coverage. (F) Quantification and (G) representative images of thrombus formation upon FeCl₃-induced injury of mesenteric arterioles in anti-mGPV mAb- or non-immune rat IgG-treated WT mice. WT (=WT+control IgG): n=15, WT+DOM3: n=11, WT+DOM/C: n=6, WT+DOM/B: n=9. #: indicates occlusive thrombus formation. (H-K) Tail bleeding time assays. Mice were injected with 100 μg DOM/B or non-immune rat IgG i.v. prior to the experiment. Mouse tails were clipped from the tail and bleeding times measured. (H) WT (with control IgG): n=8, WT+DOM/B: n=10, RhoA^{fl/fl Pf4-cre}: n=9, RhoA^{fl/fl Pf4-cre}+DOM/B: n=10. (I) WT (with control IgG): n=15, Nbeal2^{-/-}: n=15, Nbeal2^{-/-} +DOM/B: n=22. (J) WT mice were injected with 0.18 μg/g bodyweight platelet depletion antibody (R300, Emfret) to induce thrombocytopenia to 5-10% of normal counts 24 h prior to tail bleeding time assay. WT (with control IgG): n=18, Thrombocytopenia: n=9, Thrombocytopenia+DOM/B: n=11. (K) WT mice were fed with 3 mg/kg clopidogrel 48 h and 24 h before the tail bleeding experiment. n=19 (all groups). (F, H-K) Kruskal-Wallis Test followed by Dunn's multiple comparison test to compare occluded vessels. Fisher's exact test was used to compare occluded vs. non-occluded vessels. *P<0.05; **P<0.01; ***P<0.001.

FIG. 5: The anti-hGPV mAb LUM/B interferes with thrombin cleavage and accelerates fibrin formation. (A) Human platelets were incubated with the indicated antibody or control IgG_(10 μg/ml) prior to thrombin stimulation. Thrombin-mediated cleavage of GPV was assessed by flow cytometry. Mean±SD, n=4. (B) Flow cytometry reveals unaltered reactivity of LUM/B-treated platelets (10 μg/ml) upon thrombin stimulation compared to human controls. Mean±SD, n=4. (C-E) Recalcified whole blood was incubated with LUM/B IgG or LUM/B F(ab)₂ prior to perfusion over collagen/TF spots. (C) Representative images of thrombus (GPIbβ) and fibrin formation. Scale bar: 20 μm. Quantification of time to fibrin formation (D) and fibrin generation during blood flow (E) after LUM/B-treatment. SAC: surface area coverage. (D) Ctrl vs. LUM/B IgG: n=7, Shapiro-Wilk normality test, two-tailed paired t-test; Ctrl vs. LUM/B F(ab)₂: n=7, Wilcoxon matched pairs signed rank test. (E) mean±SEM. n=9. Two-tailed unpaired t-test. Ctrl: Human donor. *P<0.05; **P<0.01; ***P<0.001.

FIG. 6: Graphical abstract highlighting findings of the present invention without intending to limit the invention and without intending to be bound by theory.

FIG. 7: Flow cytometry Exemplified gating strategy based on FSC/SSC characteristics

FIG. 8: R476A point mutation renders GPV insensitive for thrombin-induced cleavage in Gp5^{dThr} mice but does not alter platelet reactivity towards thrombin. (A) Simplified targeting strategy. Gp5^{dThr} mice were generated by introduction of the point mutation R476A in the thrombin cleavage site. (B-E) Washed platelets were left untreated or stimulated with 867 pM thrombin (in the presence of 40 μg/ml integrilin and 5 μM EGTA to prevent platelet aggregation) or 2 mM N-ethylmaleimide (NEM) to induce metalloproteinase-induced shedding of GPV. (B) After stimulation, sGPV levels in the platelet supernatant were measured using a mGPV ELISA. Mean±SD. n=3, two-tailed unpaired t-test with Welch's correction. (C-E) Stimulated platelets were incubated with saturating amounts of fluorophore-conjugated antibodies for 15 min at RT and the surface expression of GPV (C), GPIb (D) and GPVI (E) was immediately analysed by flow cytometry. Mean±SD. n=3, two-tailed unpaired t-test with Welch's correction. (F) Increased αIIbβ3 integrin activation measured by JON/A-PE staining of GPV-deficient platelets at threshold thrombin concentrations. Mean±SD, n=4, two-tailed unpaired t-test with Welch's correction. (G, H) Platelet-rich plasma (PRP) was stimulated with ADP and light transmission of PRP was recorded on a Apact four-channel aggregometer over 10 min. Representative curves for n=3-4 (G) and maximum aggregation (H) expressed as mean±SD. (I) Flow cytometry reveals unaltered reactivity of Gp5^{dThr} platelets upon thrombin stimulation compared to WT controls. Mean±SD, n=4, two-tailed unpaired t-test with Welch's correction. (J) Clot formation in PRP was induced by the addition of thrombin and CaCl₂ and clot retraction was monitored over time. Residual volume of serum after clot retraction was measured. Mean±SD. n≥4 and representative images at 90 min after initiation of clot retraction. *P<0.05; **P<0.01; ***P<0.001.

FIG. 9: GPV regulates platelet responsiveness to thrombin by interference with GPIbα-dependent PAR signalling. (A) Platelets were incubated with increasing concentrations of p0p/B Fab fragments and BP-FIIa binding was assessed by flow cytometry. (B, C) The thrombin-binding site on GPIbα was blocked by p0p/B Fab and αIIbβ3 integrin activation (JON/A-PE binding) of GPV-mutant and WT platelets measured by flow cytometry upon thrombin stimulation (B, C) were measured in one experiment. Mean±SD. n=4. one-way ANOVA followed by Tukey's multiple comparisons test. (D) Scheme of GPIb-thrombin and GPV-thrombin interactions. GPIX, GPIbβ, GPIbα and GPV form a complex on the platelet surface. Thrombin binds to GPIbα via a high affinity binding site, cleaves GPV and activates platelets via PARs. The anti-GPIbα Fab p0p/B blocks the thrombin binding site on GPIbα and thereby reduces thrombin-mediated platelet activation. GPV acts is an inhibitor of GPIb-mediated PAR activation. *P<0.05; **P<0.01; ***P<0.001; ****P<0.0001.

FIG. 10: Unaltered thrombin generation in GPV mutant PRP. TF-initiated thrombin generation was measured in platelet-rich plasma (PRP) upon platelet activation. Platelets were left unstimulated (PRP) or activated by incubation with collagen-related peptide (CRP) (20 μg/ml), rhodocytin (RC, 1 μg/ml), ionomycin (10 μM) or A23187 (10 μM) for 10 min at 37° C. Thrombin generation was triggered with tissue factor/CaCl₂. Lag time (A, D), maximal thrombin concentration (B, E) and time to peak (C, F) were determined. Values are depicted as mean±SD. n 4. Two-tailed unpaired t-test with Welch's correction. *P<0.05.

FIG. 11: Absence of GPV restores thrombotic and haemostatic defects in the absence of GPVI. GPVI was depleted from the platelet surface by injection of the anti-GPVI mAb JAQ1. Confirmation of GPVI depletion by Western blot analysis (A) and flow cytometry (B). (C) Quantification and representative images (D) of thrombus formation upon FeCl₃-induced injury of mesenteric arterioles. Thrombus formation in no more than two arterioles of each mouse were analysed; data points represent measurements of one arteriole. n 12. ##compared to JAQ1-treated WT mice. *** compared to untreated WT mice. # indicates vessel occlusion. (E) The abdominal aorta was mechanically injured by a single firm compression with a forceps and blood flow was monitored with a Doppler flowmeter. Time to cessation of blood flow is shown. Each symbol represents one mouse. ###compared to untreated WT mice. n 7. (F) Haemostatic function was assessed using a tail bleeding assay on filter paper. Each symbol represents one mouse. n 9. Fisher's exact test for open vs. occluded vessels, one-way ANOVA followed by Dunn's test for multiple comparisons to compare occluded vessels. *P<0.05; **, ##P<0.01; ***, ###P<0.001.

FIG. 12: Cleaved GPV preferentially localises to fibrin adjacent to thrombus. (A) Image analysis pipeline to quantify GPV intensities (stained with AF546-labeled DOM/C) inside fibrin fibres (Fibrin(ogen) AF488) and outside the thrombus (platelets labelled with anti-GPIX derivative AF405). (B) First, GPV signal was analysed inside and outside the thrombus/fibrin. (C) GPV intensities was calculated inside fibrin but outside GPIX-positive area.

FIG. 13: rhGPV delays and reduces fibrin formation. (A) P-selectin exposure at threshold thrombin concentrations in rhGPV-treated (20 μg/ml) and WT platelets. Mean±SD of n=5 mice. (B) Unaltered αIIbβ3 integrin activation (JON/A-PE binding) at threshold thrombin concentrations in the presence of rhGPV. Mean±SD, n=5. Welch's test. (C-1) Recalcified whole blood was incubated in vitro with 20 μg/ml rhGPV prior to perfusion over collagen/TF spots. Quantification of fibrin generation during blood flow in human (C-E) and mouse blood (F, G). (B) mean±SD; n=4, Mann-Whitney test. Ctrl: human donor. (D, F) Representative images displaying decreased fibrin formation after rhGPV-treatment. Scale bar: 20 μm. (E) Time to fibrin formation in rhGPV-treated human blood. Ctrl: Human control. n=4, two-tailed unpaired t-test. (G) Time to fibrin formation in rhGPV-treated human blood. WT: n=6, WT+rhGPV: n=7, Mann-Whitney test. (H) Fibrin formation using Fibrin(ogen)A488 or the anti-Fibrin antibody 59D8. Representative images. (I) Analysis of fibrin fibrils by laser scanning confocal microscopy. Fibrin fibres were visualized using a Leica SP8 confocal microscope, 63× oil, Hyvolution mode, z-step size: 0.1 μm, 15 μm z-size. Images were deconvolved using Huygens Software. Maximum projection. Scale bar: 20 μm. Zoom-in: 5 μm. Representative of n=5 mice. (J) Thrombin activity was determined in the outflow of the flow chamber using a fluorogenic thrombin substrate and measured immediately after sample collection. mean±SD; n=7, two-tailed unpaired t-test with Welch's correction. (K) Thrombin activity in flow chambers was determined using the fluorogenic thrombin substrate Z-GGR-AMC and representative images. mean±SD; n=5, two-tailed unpaired t-test with Welch's correction. Scale bar: 20 μm. (L) Carstairs staining of the abdominal aorta after mechanical injury-induced thrombus formation in a WT and rhGPV-treated mouse (red blood cells: block arrow, fibrin: arrowhead, platelets: arrow, collagen). Scale bar: 100 μm. *P<0.05; **P<0.01; ***P<0.001. rhGPV: 20 μg/ml. SAC: surface area coverage.

FIG. 14: Unaltered MCA vessel diameter in Gp5^{-/-} mice. Optically transparent brain samples of Gp5^{-/-} and WT mice were imaged using light sheet fluorescence microscopy (LSFM). (A) Due to its conserved branching and its easy recognition, the present inventors focused on the region around the middle cerebral artery (MCA) to allow better comparability between the samples. (B) The present inventors analyzed the vessel diameter of the MCA (1) and 2 subsequent branches of the caudal (2) and rostral (5) branch of the MCA using Imaris Software. (C-E) Vessels in the left, right hemisphere and the combination of both hemispheres did not show any difference between GPV-deficient and WT mice. (F) Vessel diameter of microvessels in the brain was comparable between Gp5^{-/-} and WT mice. Mean±SD. n=4. two-tailed unpaired t-test with Welch's correction. (G) PcomA scores (posterior communicating artery), which was determined in brains from mice that were perfused with PBS followed 3 ml black ink diluted in 4% PFA (1:5 v/v). n=5. Mann-Whitney test.

FIG. 15: DOM/B does not alter platelet responses upon stimulation with collagen or thrombin in vitro, despite interfering with thrombin-mediated cleavage of GPV. (A-C) Washed platelets were incubated in vitro with 10 μg/ml of the indicated antibodies for 5 min and stimulated with Horm or soluble collagen. Light transmission was recorded on an Apact four-channel aggregometer over 20 min. Representative aggregation curves of n=3, 2 independent experiments. LEN/B: anti-α2 integrin antibody⁵¹. (D, E) Flow cytometry reveals unaltered reactivity of DOM/B-treated WT platelets upon thrombin stimulation compared to WT controls. Mean±SD. n=3, two-tailed unpaired t-test with Welch's correction. (F) Aggregation upon thrombin-stimulation is not affected in the presence of DOM/B. Light transmission was recorded on an Apact four-channel aggregometer over 10 min. Representative curves of at least 3 individual experiments. (G) Thrombin exosites I and II were blocked by the aptamers HD1 and HD22, respectively. Platelets were incubated with the indicated antibody (10 μg/ml) prior to thrombin stimulation. Thrombin-mediated cleavage of GPV was assessed by flow cytometry. Mean±SD. n≥3. (H) Thrombin clotting time was assessed using a ball coagulometer in GPV mutant or anti-mGPV mAb treated PRP in the presence or absence of HD22. Antibody concentration: 10 μg/ml; aptamer concentration: 1.5 μM f.c., thrombin: 17 nM. Mean±SD. WT, WT+DOM/B, WT+DOM/C: n=5, Gp5^{-/-}: n=4, Gp5^{dThr}, Gp5^{-/-}+HD22, Gp5^{dThr}+HD22: n=3, WT+DOM/B+HD22, WT+DOM/C+HD22: n=6, WT+HD22: n=8. One-way ANOVA followed by Dunn's test for multiple comparisons.

FIG. 16: DOM/B restores haemostasis and thrombus formation in the absence of GPVI, thereby reproducing the Gp5−/− phenotype. (A) Recalcified blood was perfused over collagen/TF spots. Thrombin activity was determined in the outflow of the flow chamber using a fluorogenic thrombin substrate and measured immediately after sample collection. Mean±SD. WT, Gp5^{-/-}, WT+DOM/B: n=7, Gp5^{dThr}: n=8, one-way ANOVA followed by Tukey's multiple comparisons test. (B) Recalcified blood was perfused over collagen/TF spots. Samples were stained for platelets (anti-GPIX derivative), fibrin(ogen) and GPV (anti-GPV derivative), fixed and analysed with a Zeiss Airyscan microscope using the superresolution mode. Quantification of GPV intensities inside fibrin-rich but non-platelet area. Background GPV intensity in Gp5^{-/-} images is displayed as dashed line. Mean±SD. 2way ANOVA followed by Tukey's multiple comparisons test, n≥11 ROI per group representing WT: n=3, WT+DOM/B: n=4 mice. (C, D) WT mice were injected with DOM/B (100 μg i.p.) and platelet count assessed for 7 days (C). Platelet count at d0 was set to 100%. Mean±SD. n=4. (D) GPV platelet surface expression was assessed by flow cytometry for up to 7 days after DOM/B injection. Mean±SD. n=4. DOM/C binds to a distinct epitope on the extracellular domain of GPV. Receptor opsonization was measured using an anti-rat IgG FITC antibody. (E-G) GPVI was depleted from the platelet surface by injection of the anti-GPVI mAb JAQ1. Representative images (E) and quantification (F) of thrombus formation upon FeCl₃-induced injury of mesenteric arterioles in WT mice after JAQ1 and DOM/B treatment. Thrombus formation in no more than two arterioles of each mouse were analysed; data points represent measurements of one arteriole. WT: n=18, WT+DOM/B: n=12, WT+JAQ1: n=13, WT+DOM/B+JAQ1: n=16, Fisher's exact test to compare occluded vs. non-occluded vessels was used. # indicates vessel occlusion. (G) Mice lacking both collagen receptors GPVI and α2 were treated with DOM/B and haemostatic function was assessed using a tail bleeding assay on filter paper. Each symbol represents one mouse. WT: n=8, Itga2^{-/-}: n=10, Itga2^{-/-}+JAQ1: n=25, Itga2^{-/-}+JAQ1+DOM/B: n=16, Fisher's exact test for open vs. occluded vessels. (H) Summary of the effects of the different anti-mGPV antibodies. n.e.: no effect. *P<0.05; **P<0.01; ***P<0.001.

FIG. 17: LUM/B has no effect on thrombin-mediated platelet activation. (A) Human platelets were incubated with 10 μg/ml LUM/B prior to flow cytometric analysis of PAC1-binding. Mean±SD, n=3. (B) Human platelets were incubated with the indicated anti-hGPV mAbs light transmission was recorded on a Apact four-channel aggregometer over 10 min. Representative curves for n=3. (C) Human platelets were incubated with the indicated antibody (10 μg/ml) prior to thrombin stimulation. Thrombin-mediated cleavage of GPV was assessed by flow cytometry. Mean±SD, n=2. (D) Flow cytometry reveals unaltered reactivity of LUM/B-treated platelets upon thrombin stimulation compared to controls. (E) Neither blockade of hGPV by LUM/B F(ab)₂ nor rhGPV (290 nM) affect thrombin time. (A, C, D, E) Values are displayed as mean±SD. (D) n=4, two-tailed unpaired t-test with Welch's correction. (E) n=3, one-way ANOVA. (F, G) Recalcified whole blood was incubated in vitro with 10 μg/ml anti-hGPV antibody LUM3 prior to perfusion over collagen/TF spots. Quantification time to fibrin formation (G) and fibrin surface coverage during blood flow of LUM3-treated and control samples (G). (G) n=6, Shapiro-Wilk normality test, two-tailed paired t-test. (G) Values are depicted as mean±SEM. Ctrl: n≥9, LUM3: n≥7, Mann-Whitney test. SAC: Surface area coverage. Ctrl: Human donor.

FIG. 18: The anti-hGPV mAb LUM11 interferes with thrombin cleavage of GPV and accelerates fibrin formation in human blood. (A) Human platelets were incubated with the indicated antibody (10 μg/ml) prior to thrombin stimulation. Thrombin-mediated cleavage of GPV was assessed by flow cytometry. Mean±SD, n=4. (B) Human platelets were incubated with the indicated antibody (2 μg/ml) prior to thrombin stimulation. Thrombin-mediated cleavage of GPV was assessed by flow cytometry. Mean±SD, n=4. (C-E) Recalcified whole blood was perfused over collagen/tissue factor (TF)-coated microspots for 6 min at a wall shear rate of 1000 s⁻¹. Time-dependent fibrin generation of LUM11-treated and human control blood was quantified. Recalcified whole blood was incubated with 10 μg/ml LUM11 prior to perfusion over collagen/TF spots. Quantification of time to fibrin formation (C) and fibrin generation during blood flow (D) after LUM 11-treatment. (E) Representative images of thrombus (anti-GPIbβ A647) and fibrin formation (fibrin(ogen) AF488). Scale bar: 20 μm. Ctrl: Human donor blood with control IgG. *P<0.05; **P<0.01; ***P<0.001.

FIG. 19: The anti-hGPV mAb LUM11 interferes with thrombin cleavage of GPV and accelerates fibrin formation in a humanized GPV mouse model. (A) hGp5^{Kin} platelets were incubated with the indicated antibody (10 μg/ml) prior to thrombin stimulation. Thrombin-mediated cleavage of GPV was assessed by flow cytometry. Mean±SD, n=5. (B-D) Recalcified whole blood was incubated with LUM11 prior to perfusion over collagen/TF spots. Quantification of time to fibrin formation (B) and fibrin generation during blood flow (C) after LUM11-treatment. (D) Representative images of thrombus (anti-GPIX A647) and fibrin formation (fibrin(ogen) AF488). Scale bar: 20 μm. Ctrl: Human donor. *P<0.05; **P<0.01; ***P<0.001.

FIG. 20: The anti-hGPV mAb LUM11 interferes with thrombin cleavage of GPV and accelerates fibrin formation in a humanized GPV mouse model. (A-C) hGp5^{KIN} mice were injected with LUM11 (100 μg i.v.) or control IgG (100 μg i.v.) and platelet count assessed for 2 days by flow cytometry. Platelet count at d0 (prior to injection) was set to 100%. (B) hGPV platelet surface expression was assessed by flow cytometry for 2 days after LUM 11 injection. (C) Receptor opsonization was measured using an anti-rat IgG FITC antibody. Mean±SD. n=5. (D, E) Tail bleeding time assays. Mice were injected with 100 μg LUM11 or non-immune rat IgG i.v. prior to the experiment. Mouse tails were clipped from the tail and bleeding times measured. (D) hGp5^{KIN} mice were injected with 0.18 μg/g bodyweight platelet depletion antibody (R300, Emfret) to induce thrombocytopenia to 5-10% of normal counts 24 h prior to tail bleeding time assay. (E) hGp5^{KIN} mice were fed with 3 mg/kg clopidogrel 48 h and 24 h before the tail bleeding experiment. Kruskal-Wallis Test followed by Dunn's multiple comparison test to compare occluded vessels (indicated by #). Fisher's exact test was used to compare occluded vs. non-occluded vessels. *P<0.05; **P<0.01; ***P<0.001.

FIG. 21: LUM11 accelerates arterial occlusive thrombus formation in hGp5^{KIN} mice after FeCl₃-induced injury of mesenteric arterioles. (A) Representative images and (B) quantification of thrombus formation upon FeCl₃-induced injury of mesenteric arterioles of LUM11-treated or control IgG-treated (100 μg i.v. each) hGp5^{KIN} mice. #: indicates occlusive thrombus formation. Data points represent measurements of one vessel. Thrombus formation in no more than two arterioles of each mouse were analysed. *P<0.05; **P<0.01; ***P<0.001.

### DETAILED DESCRIPTION

In the following, some aspects of the invention will be explained in further detail.

As defined in the claims, a first aspect herein, relates to antibodies or fragments or derivatives thereof, which are preferably characterized by certain CDRs.

Without intending to be bound by theory, antibodies of the invention (as well as their fragments and derivatives) are preferably characterized in that said antibody, fragment or derivative is capable of inhibiting thrombin-mediated cleavage of GPV. Inhibition of thrombin-mediated cleavage can for example be determined according to the following assay:

Washed platelets are adjusted to 50,000 platelets/μl in Tyrode's buffer with Ca²⁺, stimulated with thrombin (human thrombin (e.g. Sigma #10602400001)) and incubated with saturating amounts of fluorophore-conjugated antibodies to determine platelet activation or thrombin-mediated cleavage of GPV. All samples are analysed directly after addition of 500 μl PBS on a FACSCalibur (BD Biosciences, Heidelberg, Germany).

In a related aspect, there is provided an antibody or a fragment or derivative thereof, (preferably as further defined elsewhere herein) comprising (i) a V_{H} domain comprising a CDR1 having an amino acid sequence as shown in SEQ ID NO:15, a CDR2 having an amino acid sequence as shown in SEQ ID NO:16, and a CDR3 having an amino acid sequence as shown in SEQ ID NO:17, and (ii) a V_{L} domain comprising a CDR1 having an amino acid sequence in accordance with the amino acid sequence as shown in SEQ ID NO:18, a CDR2 having an amino acid sequence in accordance with the amino acid sequence as shown in SEQ ID NO:19, and a CDR3 having an amino acid sequence in accordance with the amino acid sequence as shown in SEQ ID NO:20. In particular embodiments, the V_{H} domain comprises an amino acid sequence as shown in SEQ ID NO:21, and the V_{L} domain comprises an amino acid sequence as shown in SEQ ID NO:23.

Similarly, there is further provided an antibody or a fragment or derivative thereof, (preferably as further defined elsewhere herein) competing for binding to GPV with an antibody comprising (i) a V_{H} domain comprising a CDR1 having an amino acid sequence as shown in SEQ ID NO:15, a CDR2 having an amino acid sequence as shown in SEQ ID NO:16, and a CDR3 having an amino acid sequence as shown in SEQ ID NO:17, and (ii) a V_{L} domain comprising a CDR1 having an amino acid sequence in accordance with the amino acid sequence as shown in SEQ ID NO:18, a CDR2 having an amino acid sequence in accordance with the amino acid sequence as shown in SEQ ID NO:19, and a CDR3 having an amino acid sequence in accordance with the amino acid sequence as shown in SEQ ID NO:20. In particular embodiments, the antibody, fragment or derivative specifically competes for binding to a GPV epitope bound by an antibody with the V_{H} domain comprising an amino acid sequence as shown in SEQ ID NO:21, and the V_{L} domain comprising an amino acid sequence as shown in SEQ ID NO:23.

In a further related aspect, there is provided an antibody or a fragment or derivative thereof, (preferably as further defined elsewhere herein) comprising (i) a V_{H} domain comprising a CDR1 having an amino acid sequence as shown in SEQ ID NO:1, a CDR2 having an amino acid sequence as shown in SEQ ID NO:2, and a CDR3 having an amino acid sequence as shown in SEQ ID NO:3, and (ii) a V_{L} domain comprising a CDR1 having an amino acid sequence in accordance with the amino acid sequence as shown in SEQ ID NO:4, a CDR2 having an amino acid sequence in accordance with the amino acid sequence as shown in SEQ ID NO:5, and a CDR3 having an amino acid sequence in accordance with the amino acid sequence as shown in SEQ ID NO:6. Preferably, the V_{H} domain comprises an amino acid sequence as shown in SEQ ID NO:7, and the V_{L} domain comprises an amino acid sequence as shown in SEQ ID NO:9.

Similarly, there is further provided an antibody or a fragment or derivative thereof, (preferably as further defined elsewhere herein) competing for binding to GPV with an antibody comprising (i) a V_{H} domain comprising a CDR1 having an amino acid sequence as shown in SEQ ID NO:1, a CDR2 having an amino acid sequence as shown in SEQ ID NO:2, and a CDR3 having an amino acid sequence as shown in SEQ ID NO:3, and (ii) a V_{L} domain comprising a CDR1 having an amino acid sequence in accordance with the amino acid sequence as shown in SEQ ID NO:4, a CDR2 having an amino acid sequence in accordance with the amino acid sequence as shown in SEQ ID NO:5, and a CDR3 having an amino acid sequence in accordance with the amino acid sequence as shown in SEQ ID NO:6. Preferably, the antibody, fragment or derivative specifically competes for binding to a GPV epitope bound by an antibody with the V_{H} domain comprising an amino acid sequence as shown in SEQ ID NO:7, and the V_{L} domain comprising an amino acid sequence as shown in SEQ ID NO:9.

In another aspect, there is provided an antibody or a fragment or derivative thereof, which comprises a V_{L} domain comprising a CDR1 having an amino acid sequence as shown in SEQ ID NO: 4 or 18, wherein one amino acid of said sequence SEQ ID NO: 4 respectively 18 may be substituted, in particular the first amino acid (R respectively K) may be substituted, a CDR2 having an amino acid sequence as shown in SEQ ID NO: 5 or 19, wherein one amino acid of said sequence SEQ ID NO: 5 respectively 19 may be substituted, in particular the first amino acid (S respectively N) may be substituted, and comprising a CDR3 having an amino acid sequence as shown in SEQ ID NO:6 and 20. In another aspect, there is provided an antibody or a fragment or derivative thereof, which comprises a V_{L} domain comprising a CDR1 having an amino acid sequence as shown in SEQ ID NO: 18, wherein the first amino acid of sequence SEQ ID NO: 18 may optionally be substituted, a CDR2 having an amino acid sequence as shown in SEQ ID NO: 19, wherein the first amino acid of sequence SEQ ID NO: 19 may optionally be substituted, and comprising a CDR3 having an amino acid sequence as shown in SEQ ID NO:20. In another aspect, the antibody comprises a V_{L} domain comprising a CDR1 having an amino acid sequence as shown in SEQ ID NO: 18, wherein the first amino acid K may optionally be substituted by R, a CDR2 having an amino acid sequence as shown in SEQ ID NO: 19, wherein the first amino acid N may optionally be substituted by S, and a CDR3 having an amino acid sequence as shown in SEQ ID NO:20. In another aspect, the antibody comprises a V_{L} domain comprising a CDR1 having an amino acid sequence as shown in SEQ ID NO: 4 or 18, a CDR2 having an amino acid sequence as shown in SEQ ID NO: 5 or 19, and a CDR3 having an amino acid sequence as shown in SEQ ID NO:20. LUM11 and LUM/B are examples of such antibodies.

### Antibodies

The term “antibody”, as used herein, is not particularly limited. In particular, the term refers to an immunoglobulin molecule that binds to or is immunologically reactive with a particular antigen, and includes polyclonal, monoclonal, genetically engineered and otherwise modified forms of antibodies including, but not limited to, chimeric antibodies, humanized antibodies, human antibodies, heteroconjugate antibodies (e.g. bispecific antibodies, diabodies, triabodies, and tetrabodies), single-domain antibodies (nanobodies) and antigen binding fragments of antibodies, including e.g. Fab′, F(ab′)2, Fab, Fv, rIgG, and scFv fragments.

Moreover, generally herein, the term “antibody”, and particularly also the “monoclonal antibody” (mAb) is meant to include both intact molecules and fragments thereof.

Consequently, antibody fragments (such as, for example, Fab and F(ab′)2 fragments) which are capable of binding to a respective antigen, are particularly also envisaged herein. Fab and F(ab′)2 fragments lack the Fc fragment of intact antibody, clear more rapidly from the circulation of the animal, and may have less non-specific tissue binding than an intact antibody⁶⁷.

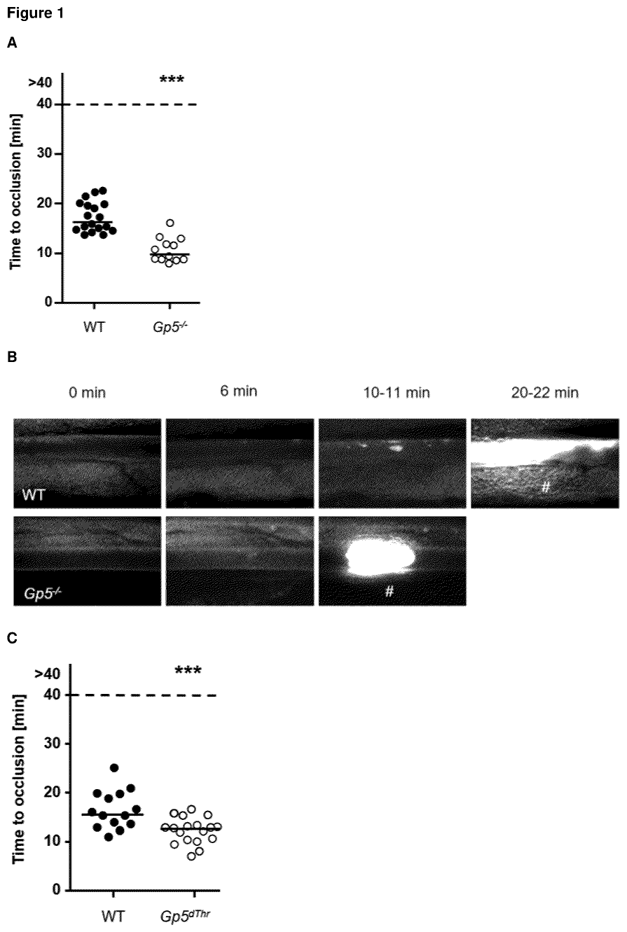

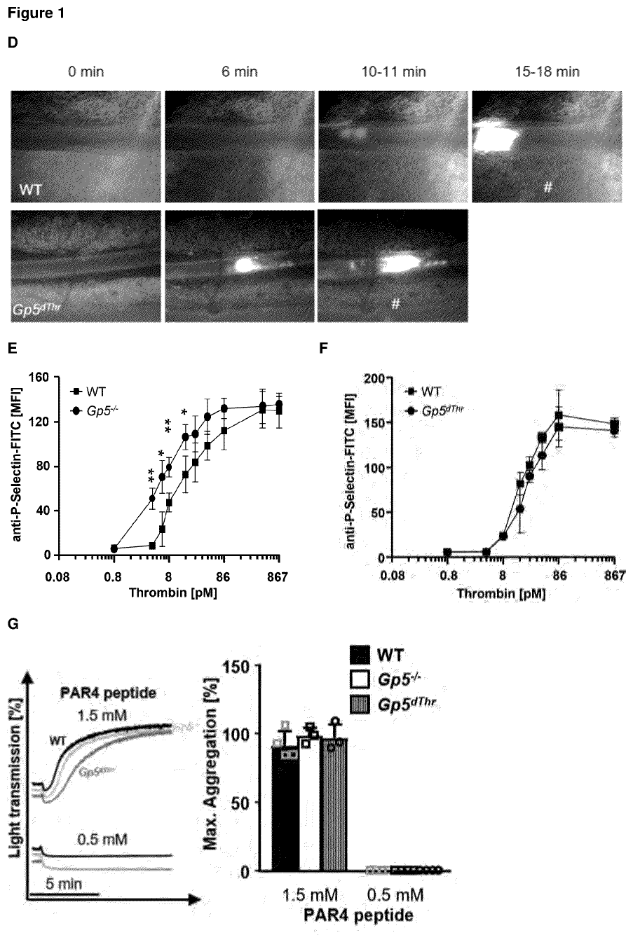

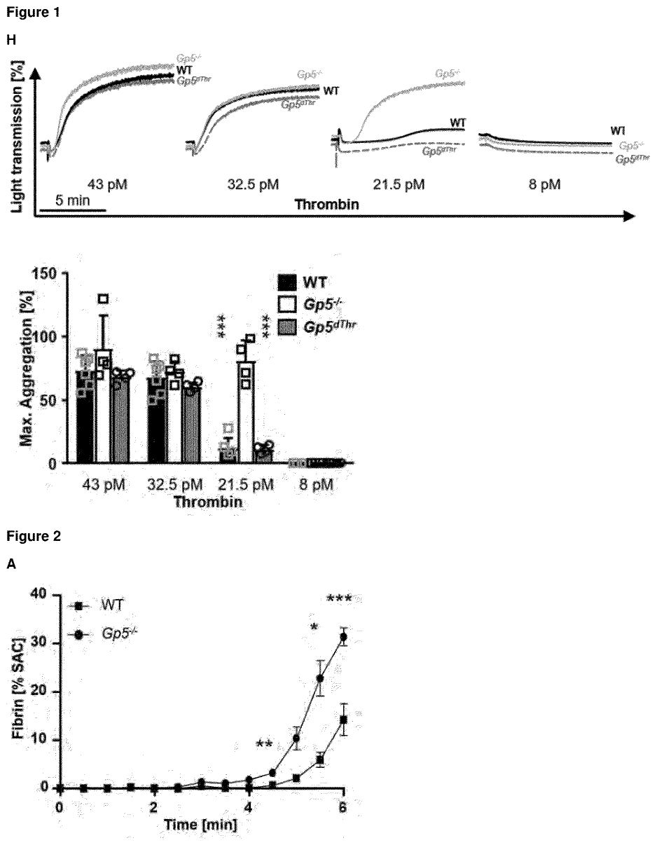

Typically, the antibody or fragment thereof, respectively, is capable of binding to the extracellular domain of GPV, preferably of human GPV. Whether a molecule or an antibody is capable of binding to the extracellular domain of GPV can be determined by a binding assay, e.g. described in Example/FIG. 1A or 1B of WO2017/109180, which is incorporated herein by reference.

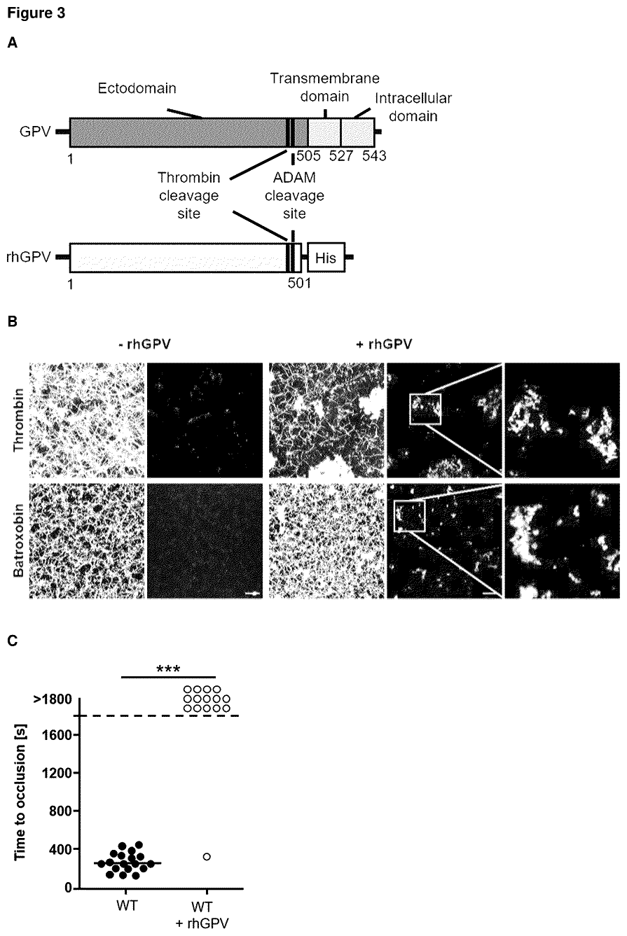

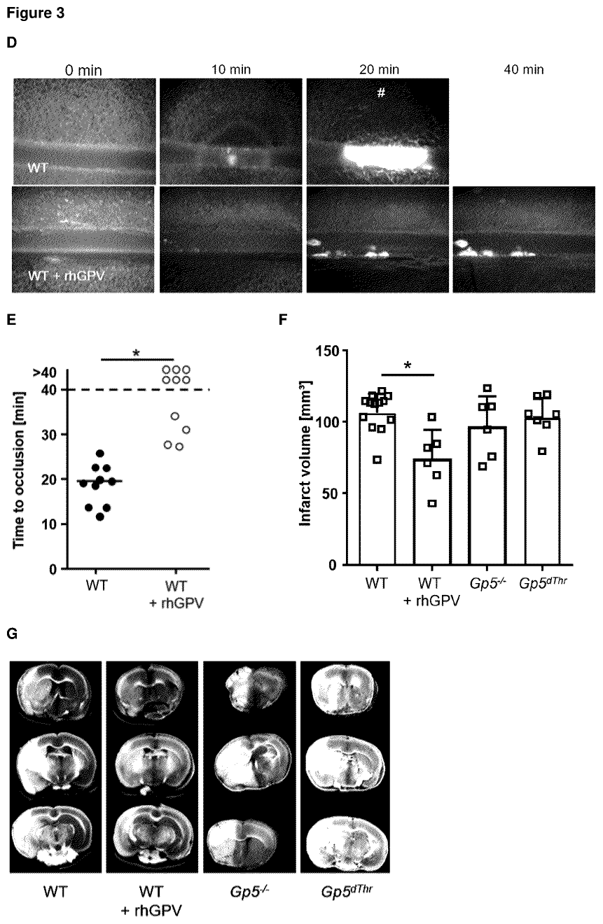

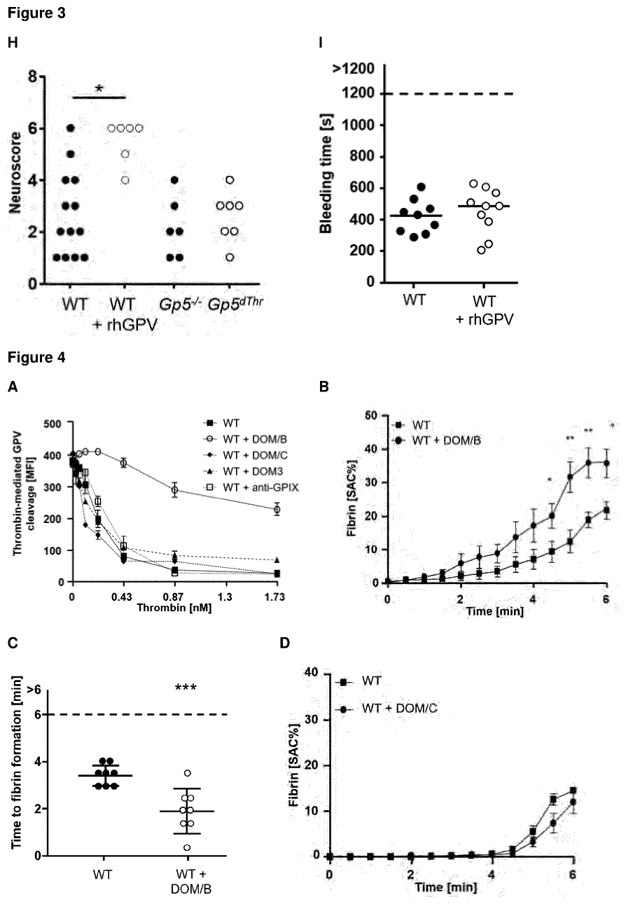

The antibody or fragment thereof referred to herein preferably is capable of binding to a region within the extracellular domain of GPV which is distinct from the collagen-binding site of GPV. In another embodiment, the antibody, fragment or derivative does not delay collagen-induced aggregation. This can be determined in an aggregation assay as described in the Examples (see FIG. 3 and materials and methods of WO2017/109180).

The dissociation constant K_{D} for the complex formed by the extracellular domain of GPV and antibody is preferably less than 100 μM, more preferably less than 10 μM, most preferably less than 5 μM. Typically the K_{D} ranges from about 1 μM to about 10 μM, or from about 10 μM to about 1 μM, or from about 100 μM to about 100 nM. Preferably, the antibody-GPV complex has a K_{D} in the range from 5 μM to 1 nM, most preferably from 10 μM to 500 μM.

Preferably, the antibody is a monoclonal antibody. The term “monoclonal antibody” as used herein is not limited to antibodies produced through hybridoma technology. The term “monoclonal antibody” refers to an antibody that is derived from a single clone, including any eukaryotic, prokaryotic, or phage clone, and not the method by which it is produced. Monoclonal antibodies can be prepared using a wide variety of techniques known in the art including the use of hybridoma, recombinant, and phage display technologies, or a combination thereof⁶⁸.

In other embodiments, including in vivo use of the antibodies in humans, chimeric, primatized, humanized, or human antibodies can be used. In a preferred embodiment, the antibody is a human antibody or a humanized antibody, more preferably a monoclonal human antibody or a monoclonal humanized antibody.

The term “chimeric” antibody as used herein refers to an antibody having variable sequences derived from non-human immunoglobulins, such as rat or mouse antibodies, and human immunoglobulins constant regions, typically chosen from a human immunoglobulin template. Methods for producing chimeric antibodies are known in the art⁶⁹⁻⁷¹, see, also U.S. Pat. Nos. 5,807,715; 4,816,567; and 4,816,397, which are incorporated herein by reference in their entireties.

“Humanized” forms of non-human (e.g. murine) antibodies are chimeric immunoglobulins, immunoglobulin chains or fragments thereof (such as Fv, Fab, Fab′, F(ab′)2 or other target-binding subsequences of antibodies), which contain minimal sequences derived from a non-human immunoglobulin. In general, the humanized antibody will comprise substantially all of at least one and typically two variable domains, in which all or substantially all of the complementarity determining regions (CDRs) correspond to those of a non-human immunoglobulin and all or substantially all of the framework (FR) regions are those of a human immunoglobulin consensus sequence. The humanized antibody can also comprise at least a portion of an immunoglobulin constant region (Fc), typically that of a human immunoglobulin template chosen. Humanization is a technique for making a chimeric antibody in which one or more amino acids or portions of the human variable domain have been substituted by the corresponding sequence from a non-human species. Humanized antibodies are antibody molecules generated in a non-human species that bind the desired antigen having one or more CDRs from the non-human species and FRs from a human immunoglobulin molecule. Often, framework residues in the human framework regions will be substituted with the corresponding residue from the CDR donor antibody to alter, preferably improve, antigen binding. These framework substitutions are identified by methods well known in the art, e.g. by modeling of the interactions of the CDR and framework residues to identify framework residues important for antigen binding and sequence comparison to identify unusual framework residues at particular positions⁷², see also e.g. and Queen et al. U.S. Pat. Nos. 5,530,101; 5,585,089; 5,693,761; 5,693,762; and 6,180,370 (each of which is incorporated by reference in its entirety). Antibodies can be humanized using a variety of techniques known in the art including, for example, CDR-grafting (EP239400; WO 91/09967; U.S. Pat. Nos. 5,225,539; 5,530,101 and 5,585,089), veneering or resurfacing⁷³⁻⁷⁵ (also EP0592106; EP0519596; and chain shuffling (U.S. Pat. No. 5,565,332)), all of which are hereby incorporated by reference in their entireties.

In some embodiments, humanized antibodies are prepared as described in Queen et al., U.S. Pat. Nos. 5,530,101; 5,585,089; 5,693,761; 5,693,762; and 6,180,370 (each of which is incorporated by reference in its entirety).

In some embodiments, the antibodies are human antibodies. Completely “human” antibodies can be desirable for therapeutic treatment of human patients. As used herein, “human antibodies” include antibodies having the amino acid sequence of a human immunoglobulin and include antibodies isolated from human immunoglobulin libraries or from animals transgenic for one or more human immunoglobulin and that do not express endogenous immunoglobulins. Human antibodies can be made by a variety of methods known in the art including phage display methods described above using antibody libraries derived from human immunoglobulin sequences. See U.S. Pat. Nos. 4,444,887 and 4,716,111; and WO 98/46645; WO 98/50433; WO 98/24893; WO 98/16654; WO 96/34096; WO 96/33735; and WO 91/10741, each of which is incorporated herein by reference in its entirety. Human antibodies can also be produced using transgenic mice which are incapable of expressing functional endogenous immunoglobulins, but which can express human immunoglobulin genes. See, e.g. WO 98/24893; WO 92/01047; WO 96/34096; WO 96/33735; U.S. Pat. Nos. 5,413,923; 5,625,126; 5,633,425; 5,569,825; 5,661,016; 5,545,806; 5,814,318; 5,885,793; 5,916,771; and 5,939,598, which are incorporated by reference herein in their entireties. Completely human antibodies that recognize a selected epitope can be generated using a technique referred to as “guided selection.” In this approach a selected non-human monoclonal antibody, e.g. a mouse antibody, is used to guide the selection of a completely human antibody recognizing the same epitope⁷⁶.

In some embodiments, the antibodies are primatized antibodies. The term “primatized antibody” refers to an antibody comprising monkey variable regions and human constant regions. Methods for producing primatized antibodies are known in the art. See e.g. U.S. Pat. Nos. 5,658,570; 5,681,722; and 5,693,780, which are incorporated herein by reference in their entireties.

### Antibody Derivatives

Further embodiments herein relate to derivatives of the antibodies (or fragments thereof, respectively) of the invention. For example, but not by way of limitation, suitable antibody derivatives include antibodies that have been modified, e.g. by glycosylation, acetylation, pegylation, phosphorylation, amidation, derivatization by known protecting/blocking groups, proteolytic cleavage or linkage to a cellular ligand or other proteins (see below for a discussion of antibody conjugates). Any of numerous chemical modifications may be carried out by known techniques, including, but not limited to, specific chemical cleavage, acetylation, formylation or metabolic synthesis of tunicamycin, etc. Additionally, the derivative may contain one or more non-classical amino acids.

In addition, in some embodiments, antibody derivatives thereof can be those, whose sequence has been modified to reduce at least one constant region-mediated biological effector function relative to the corresponding wild type sequence. To modify an antibody, such that it exhibits reduced binding to the Fc receptor, the immunoglobulin constant region segment of the antibody can be mutated at particular regions necessary for Fc receptor (FcR) interactions (e.g. ⁷⁷⁻⁷⁸). Reduction in FcR binding ability of the antibody can also reduce other effector functions which rely on FcR interactions, such as opsonization, phagocytosis and antigen-dependent cellular cytotoxicity.

In yet another aspect, antibody derivatives thereof can be those that have been modified to increase or reduce their binding affinities to the fetal Fc receptor, FcRn. To alter the binding affinity to FcRn, the immunoglobulin constant region segment of the antibody can be mutated at particular regions necessary for FcRn interactions (see, e.g. WO 2005/123780). Increasing the binding affinity to FcRn should increase the antibody's serum half-life, and reducing the binding affinity to FcRn should conversely reduce the antibody's serum half-life. Specific combinations of suitable amino acid substitutions are identified in Table 1 of WO 2005/123780, which table is incorporated by reference herein in its entirety. See also, Hinton et al., U.S. Pat. Nos. 7,217,797, 7,361,740, 7,365,168, and 7,217,798, which are incorporated herein by reference in their entireties.

In yet other aspects, an antibody derivative has one or more amino acids inserted into one or more of its hypervariable regions, for example as described in US 2007/0280931.

### Antibody Conjugates

In some embodiments, the antibodies of the invention or their derivatives, respectively, are antibody conjugates that are modified, e.g. by the covalent attachment of any type of molecule to the antibody, such that covalent attachment preferably does not interfere with antigen binding. Techniques for conjugating effector moieties to antibodies are well known in the art (e.g. ⁷⁹⁻⁸¹).

In one example, the antibody or fragment thereof is fused via a covalent bond (e.g. a peptide bond), at optionally the N-terminus or the C-terminus, to an amino acid sequence of another protein (or portion thereof; preferably at least a 10, 20 or 50 amino acid portion of the protein). Preferably, the antibody or fragment thereof is linked to the other protein at the N-terminus of the constant domain of the antibody. Recombinant DNA procedures can be used to create such fusions, for example as described in WO 86/01533 and EP 0392745. In another example, the effector molecule can increase half-life in vivo. Examples of suitable effector molecules of this type include polymers, albumin, albumin binding proteins or albumin binding compounds, such as those described in WO 2005/117984.

In some embodiments, the antibodies can be attached to poly(ethyleneglycol) (PEG) moieties. For example, if the antibody is an antibody fragment, the PEG moieties can be attached through any available amino acid side-chain or terminal amino acid functional group located in the antibody fragment, for example any free amino, imino, thiol, hydroxyl or carboxyl group. Such amino acids can occur naturally in the antibody fragment or can be engineered into the fragment using recombinant DNA methods. See, for example U.S. Pat. No. 5,219,996. Multiple sites can be used to attach two or more PEG molecules. Preferably, PEG moieties are covalently linked through a thiol group of at least one cysteine residue located in the antibody fragment. Where a thiol group is used as the point of attachment, appropriately activated effector moieties, for example thiol selective derivatives, such as maleimides and cysteine derivatives, can be used.

In another example, an antibody conjugate is a modified Fab′ fragment which is PEGylated, i.e., has PEG (poly(ethyleneglycol)) covalently attached thereto, e.g. according to the method disclosed in EP 0948544 (see also ⁸²⁻⁸⁵).

Further preferred antibodies are antibodies, fragments and derivatives thereof which compete with the specific antibodies of the present invention for binding to human GPV. Further preferred inhibitors are antibodies, fragments and derivatives thereof which bind to an epitope on GPV which overlaps with the epitope on GPV of the specific antibodies of the present invention. Further preferred antibodies herein are antibodies, fragments and derivatives thereof which bind to the same epitope on GPV as the specific antibodies of the present invention.

In certain embodiments herein, the antibody, fragment or derivative of the invention is not antibody 89F12 (disclosed in WO2017/109180).

In certain embodiments herein, the antibody, fragment or derivative of the invention is not antibody 89H111 (disclosed in WO2017/109180).

In certain embodiments herein, the antibody, fragment or derivative of the invention is not antibV.3⁸⁶⁻⁸⁷.

The embodiments in the preceding three paragraphs may particularly be combined with each other.

### Glycoprotein V (GPV)

The term “Glycoprotein V” or “GPV”, as used interchangeably herein, denotes a membrane protein having a sequence identity of at least 50% to the amino acid sequence as shown in SEQ ID NO: 31. Preferably, the GPV has an amino acid identity of at least 60%, or at least 70%, or at least 80%, such as at least 90%, in particular at least 95% to the amino acid sequence as shown in SEQ ID NO: 31.

The GPV referred to herein typically is platelet glycoprotein V and has a functional transmembrane domain. Typically, the GPV is a naturally occurring GPV. Preferably, the GPV is of mammalian origin. Most preferably, the GPV is a human GPV. According to this embodiment, the GPV preferably comprises or consists of the amino acid sequence as shown in SEQ ID NO: 31.

Besides, in embodiments herein referring to GPV in general (i.e. not in the context of the cleavage of GPV), GPV may also be “soluble GPV” or “sGPV”, as used interchangeably herein—wherein the skilled reader will appreciate that sGPV may result from the cleavage of GPV by thrombin.

### Thrombin

Thrombin is well known to the skilled person as an important enzyme (more particularly a serine protease) in haemostasis. It is capable of converting fibrinogen to fibrin. Moreover, thrombin is capable of cleaving GPV as will be readily understood by a person skilled in the art.

### Percent Identity

In accordance with the present invention, a sequence being evaluated (the “Compared Sequence”) has a certain “percent identity with”, or is certain “percent identical to” a claimed or described sequence (the “Reference Sequence”) after alignment of the two sequences. The “Percent Identity” is determined according to the following formula:

$$Percent Identity = 100 [ 1 - ( C / R ) ]$$

In this formula, C is the number of differences between the Reference Sequence and the Compared Sequence over the length of alignment between the two sequences wherein (i) each base in the Reference Sequence that does not have a corresponding aligned base in the Compared Sequence, and (ii) each gap in the Reference Sequence, and (iii) each aligned base in the Reference Sequence that is different from an aligned base in the Compared Sequence constitutes a difference. R is the number of bases of the Reference Sequence over the length of the alignment with the Compared Sequence with any gap created in the Reference Sequence also being counted as a base.

If an alignment exists between the Compared Sequence and the Reference Sequence for which the Percent Identity (calculated as above) is about equal to, or greater than, a specified minimum, the Compared Sequence has that specified minimum Percent Identity even if alignments may exist elsewhere in the sequence that show a lower Percent Identity than that specified.

In a preferred embodiment, the length of aligned sequence for comparison purposes is at least 30%, preferably at least 40%, more preferably at least 50%, even more preferably at least 60%, and even more preferably at least 70%, 80%, or 90% of the length of the Reference Sequence.

The comparison of sequences and determination of percent identity (and percent similarity) between two amino acid sequences can be accomplished using any suitable program, e.g. the program “BLAST 2 SEQUENCES (blastp)”⁸⁸ with the following parameters: Matrix BLOSUM62; Open gap 11 and extension gap 1 penalties; gap x_dropoff50; expect 10.0 word size 3; Filter: none. According to the present invention, the sequence comparison covers at least 40 amino acids, preferably at least 80 amino acids, more preferably at least 100 amino acids, and most preferably at least 120 amino acids.

### Nucleic Acids

A further aspect of the invention relates to nucleic acids encoding the antibody or fragment or derivative thereof. Preferred embodiments of this aspect correspond to preferred embodiments described herein in context with the said antibody or fragment or derivative thereof.

### Inhibitors of the Invention

The antibodies, derivatives, fragments and nucleic acids of the invention may also be referred to herein as an “inhibitor of the invention” (and particularly also as a “GPV inhibitor”). Likewise, the antibodies, derivatives, fragments and nucleic acids of the invention may also be referred to as an “antibody or another inhibitor of the invention”.

Consequently, as used herein, an inhibitor of the invention is a compound which preferably (i) has pro-coagulant activity and/or (ii) is capable of binding to the extracellular domain of GPV. Preferably, the inhibitor is a compound which (i) has pro-coagulant activity and (ii) is capable of binding to the extracellular domain of GPV.

In accordance with this invention, pro-coagulant activity may be determined in a “Bleeding Time Assay” as described in the examples, with the proviso that the mouse used in the Bleeding Time Assay is a transgenic mouse lacking endogenous GPV and expressing human GPV.

In accordance with this invention, binding to the extracellular domain of GPV can be determined by flow cytometry or in an ELISA as described in the examples.

The type or class of the inhibitor is not particularly limited. Preferably, however, the compound is an antibody or a fragment thereof. In yet another embodiment, the GPV inhibitor is a nucleic acid.

Generally, and as outlined above, the antibody or another inhibitor of the invention is preferably capable of interfering with thrombin cleavage of GPV. In other words, an antibody or another inhibitor of the invention may affect, e.g. inhibit, the thrombin-mediated cleavage of GPV, particularly in a subject upon administration of the inhibitor to the subject.

### Haemorrhagic Conditions

The antibody or another inhibitor of the invention described herein is preferably used in the treatment or prevention of a haemorrhagic condition. Haemorrhagic conditions are characterized by excessive bleeding. The excessive bleeding can have various causes. In some embodiments the haemorrhagic condition is a haemorrhagic disease associated with a prolonged bleeding time.

In one embodiment, the haemorrhagic condition is caused by a platelet disorder.

The platelet disorder may be characterized by a decreased number of platelets, e.g. in the case of thrombocytopenia. Specific thrombocytopenias include, but are not limited to, idiopathic thrombocytopenic purpura, thrombotic thrombocytopenic purpura, drug-induced thrombocytopenia due to immune-mediated platelet destruction (e.g. by heparin, trimethoprim/sulfamethoxazole), drug-induced thrombocytopenia due to dose-dependent bone marrow suppression (e.g. by chemotherapeutic agents), thrombocytopenia accompanying systemic infection, thrombocytopenia caused by chemotherapy, gestational thrombocytopenia, and immune thrombocytopenia (ITP, formerly called immune thrombocytopenic purpura).

The platelet disorder may be characterized by a dysfunction of the platelets, e.g. in the case of defective platelet signaling due to lack of platelet receptors or signaling molecules.

Alternatively, in some embodiments the haemorrhagic condition instead of being a haemorrhagic disease associated with a prolonged bleeding time may be caused by a previous intervention with anti-platelet and/or anti-coagulant medication resulting in a prolonged bleeding time. Such a haemorrhagic condition may be undesirable, for example, as it may increase the risk of an emergency surgery (e.g. after a car accident) or it may be associated with an overdose of anti-platelet and/or anti-coagulant medication. In some of said embodiments the haemorrhagic condition is not a haemorrhagic disease.

In a preferred embodiment, the haemorrhagic condition is selected from the group consisting of inflammatory bleeding, haemophilia/FVIII, bleeding due to anti-platelet therapy, bleeding due to anti-coagulant therapy, haemorrhagic stroke, excessive bleeding due to sepsis, excessive bleeding due to thrombocytopenia, excessive bleeding due to disseminated intravascular coagulation (DIC), excessive bleeding due to chemotherapy, excessive bleeding due to haemolytic-uremic syndrome, and excessive bleeding due to HIV infection.

In a specific embodiment, the present invention relates to the use of the antibody or another inhibitor of the invention described herein as antidote for the administration of soluble GPV.

### Pharmaceutical Compositions and Treatment

Treatment of a disease encompasses the treatment of patients already diagnosed as having any form of the disease at any clinical stage or manifestation; the delay of the onset or evolution or aggravation or deterioration of the symptoms or signs of the disease; and/or preventing and/or reducing the severity of the disease.

A “subject” or “patient” to whom an antibody or another inhibitor of the invention is administered may be a mammal, such as a non-primate (e.g. cow, pig, horse, cat, dog, rat, etc.) or a primate (e.g. monkey or human). In certain aspects, the human is a pediatric patient. In other aspects, the human is an adult patient.

The antibody, fragment, or derivative of this disclosure may be used in medicine, particularly for use in improving, preferably restoring, haemostasis. In some embodiments, it may be used in the treatment or prevention of a haemorrhagic condition. Such a condition may be due to/caused by a platelet disorder, especially wherein said platelet disorder is characterized by a decreased number of platelets and/or ii) said use in medicine is due to a condition selected from or said haemorrhagic condition is selected from the group consisting of inflammatory bleeding, haemophilia/FVIII, bleeding due to anti-platelet therapy, bleeding due to anti-coagulant therapy, haemorrhagic stroke, excessive bleeding due to sepsis, excessive bleeding due to thrombocytopenia, excessive bleeding due to disseminated intravascular coagulation (DIC), excessive bleeding due to chemotherapy, excessive bleeding due to haemolytic-uremic syndrome, excessive bleeding upon administration of soluble GPV, and excessive bleeding due to HIV infection.

In some embodiments, the antibody, fragment, or derivative of this disclosure may be used for treatment that is reversing the effect of anti-platelet and/or anti-coagulant medication, for example reversing the effect of anti-platelet medication and/or anti-coagulant medication in emergency bleeding control. In some embodiments the effect of anti-platelet medication and/or anti-coagulant medication is reversed, wherein the anti-platelet medication and/or anti-coagulant medication is selected from a group consisting of aspirin, ADP receptor inhibitors (such as Clopidogrel, Prasugrel or Ticagrelor), anti-GPVI treatment (e.g. Glencozimab), spleen tyrosine kinase inhibitors (e.g. Fostamatinib), Bruton's tyrosin kinase inhibitors, GPIbα inhibitors (e.g. Volociximab), dipyridamole or protease-activated receptor-1 inhibitors (e.g. Vorapaxar), warfarin, heparin, low molecular weight heparin (LMWH, such as enoxaparin, dalteparin or tinzaparin), activators of antithrombin III (such as fondaparinux), thrombin inhibitors (e.g. dabigatran) or inhibitors of factor Xa (such as Rivaroxaban, Edoxaban or Apixaban). In some embodiments the effect of, in particular anti-platelet, medication is reversed, wherein the medication is selected from a group consisting of aspirin, ADP receptor inhibitors (such as Clopidogrel, Prasugrel or Ticagrelor), anti-GPVI treatment (e.g. Glencozimab), spleen tyrosine kinase inhibitors (e.g. Fostamatinib), Bruton's tyrosin kinase inhibitors, GPIbα inhibitors (e.g. Volociximab), dipyridamole or protease-activated receptor-1 inhibitors (e.g. Vorapaxar). In some embodiments the effect of, in particular anti-coagulant, medication is reversed, wherein the medication is selected from a group consisting of warfarin, heparin, low molecular weight heparin (LMWH, such as enoxaparin, dalteparin or tinzaparin), activators of antithrombin III (such as fondaparinux), thrombin inhibitors (e.g. dabigatran) or inhibitors of factor Xa (such as Rivaroxaban, Edoxaban or Apixaban). In particularly suitable embodiments, the antibody, fragment, or derivative of this disclosure may be used for treatment that is reversing the effect of ADP receptor inhibitors, anti-GPVI treatment, spleen tyrosine kinase inhibitors.

Pharmaceutical compositions comprising an antibody or another inhibitor of the invention and optionally one or more additional therapeutic agents, such as the second therapeutic agents described below, are described herein. The compositions typically are supplied as part of a sterile, pharmaceutical composition that includes a pharmaceutically acceptable carrier. This composition can be in any suitable form (depending upon the desired method of administering it to a patient).

The antibody or another inhibitor of the invention can be administered to a patient by a variety of routes such as orally, transdermally, subcutaneously, intranasally, intravenously, intramuscularly, intrathecally, topically or locally, in particular subcutaneously or intravenously. The most suitable route for administration in any given case will depend on the particular antibody, the subject, and the nature and severity of the disease and the physical condition of the subject. Typically, an antibody or another inhibitor of the invention will be administered intravenously.

Another aspect of the invention is a pharmaceutical composition comprising the antibody or antigen-binding fragment thereof of the invention. The antibody or antigen-binding fragment thereof can be formulated according to known methods for preparing a pharmaceutical composition. For example, it can be mixed with one or more pharmaceutically acceptable carriers, diluents or excipients. For example, sterile water or physiological saline may be used. Other substances, such as pH buffering solutions, viscosity reducing agents, or stabilizers may also be included.

A wide variety of pharmaceutically acceptable excipients and carriers are known in the art. Such pharmaceutical carriers and excipients as well as suitable pharmaceutical formulations have been amply described in a variety of publications (see for example “Pharmaceutical Formulation Development of Peptides and Proteins”, Frokjaer et al., Taylor & Francis (2000) or “Handbook of Pharmaceutical Excipients”, 3rd edition, Kibbe et al., Pharmaceutical Press (2000) A. Gennaro (2000) “Remington: The Science and Practice of Pharmacy”, 20th edition, Lippincott, Williams, & Wilkins; Pharmaceutical Dosage Forms and Drug Delivery Systems (1999) H. C. Ansel et al., eds 7th ed., Lippincott, Williams, & Wilkins; and Handbook of Pharmaceutical Excipients (2000) A. H. Kibbe et al., eds., 3rd ed. Amer. Pharmaceutical Assoc). In particular, the pharmaceutical composition comprising the antibody of the invention may be formulated in lyophilized or stable soluble form. The polypeptide may be lyophilized by a variety of procedures known in the art. Lyophilized formulations are reconstituted prior to use by the addition of one or more pharmaceutically acceptable diluents such as sterile water for injection or sterile physiological saline solution.

The pharmaceutical composition of the invention can be administered in dosages and by techniques well known in the art. The amount and timing of the administration will be determined by the treating physician or veterinarian to achieve the desired purposes. The route of administration can be via any route that delivers a safe and therapeutically effective dose to the blood of the subject to be treated. Possible routes of administration include systemic, topical, enteral and parenteral routes, such as intravenous, intraarterial, subcutaneous, intradermal, intraperitoneal, oral, transmucosal, epidural, or intrathecal. Preferred routes are intravenous or subcutaneous.

The effective dosage and route of administration are determined by factors, such as age and weight of the subject, and by the nature and therapeutic range of the antibody or antigen-binding fragment thereof. The determination of the dosage is determined by known methods, no undue experimentation is required.

A therapeutically effective dose is a dose of the antibody or antigen binding fragment thereof of the invention that brings about a positive therapeutic effect in the patient or subject requiring the treatment. A therapeutically effective dose is in the range of about 0.01 to 50 mg/kg, from about 0.01 to 30 mg/kg, from about 0.1 to 30 mg/kg, from about 0.1 to 10 mg/kg, from about 0.1 to 5 mg/kg, from about 1 to 5 mg/kg, from about 0.1 to 2 mg/kg or from about 0.1 to 1 mg/kg. The treatment may comprise giving a single (e.g. bolus) dose or multiple doses. Alternatively continuous administration is possible. If multiple doses are required, they may be administered daily, every other day, weekly, biweekly, monthly, or bimonthly or as required. A depository may also be used that slowly and continuously releases the antibody or antigen-binding fragment thereof. A therapeutically effective dose may be a dose that inhibits GPV in the subject by at least 50%, preferably by at least 60%, 70%, 80%, 90%, more preferably by at least 95%, 99% or even 100%.

The antibody can be formulated as an aqueous solution.

Pharmaceutical compositions can be conveniently presented in unit dose forms containing a predetermined amount of an antibody or another inhibitor of the invention, per dose. Such a unit can contain 0.5 mg to 5 g, for example, but without limitation, 1 mg, 10 mg, 20 mg, 30 mg, 40 mg, 50 mg, 100 mg, 200 mg, 300 mg, 400 mg, 500 mg, 750 mg, 1000 mg, or any range between any two of the foregoing values, for example 10 mg to 1000 mg, 20 mg to 50 mg, or 30 mg to 300 mg. Pharmaceutically acceptable carriers can take a wide variety of forms depending, e.g. on the condition to be treated or route of administration.

Determination of the effective dosage, total number of doses and length of treatment with an antibody or another inhibitor of the invention is well within the capabilities of those skilled in the art and can be determined using a standard dose escalation study.

Therapeutic formulations of the an antibody or another inhibitor of the invention, suitable in the methods described herein can be prepared for storage as lyophilized formulations or aqueous solutions by mixing the inhibitor, e.g. the antibody, having the desired degree of purity with optional pharmaceutically-acceptable carriers, excipients or stabilizers typically employed in the art (all of which are referred to herein as “carriers”), i.e. buffering agents, stabilizing agents, preservatives, isotonifiers, non-ionic detergents, antioxidants and other miscellaneous additives. See, Remington's Pharmaceutical Sciences, 16th edition (Osol, ed. 1980). Such additives must be nontoxic to the recipients at the dosages and concentrations employed.

Buffering agents help to maintain the pH in the range which approximates physiological conditions. They can be present at concentrations ranging from about 2 mM to about 50 mM. Suitable buffering agents include both organic and inorganic acids and salts thereof, such as citrate buffers (e.g. monosodium citrate-disodium citrate mixture, citric acid-trisodium citrate mixture, citric acid-monosodium citrate mixture, etc.), succinate buffers (e.g. succinic acid-monosodium succinate mixture, succinic acid-sodium hydroxide mixture, succinic acid-disodium succinate mixture, etc.), tartrate buffers (e.g. tartaric acid-sodium tartrate mixture, tartaric acid-potassium tartrate mixture, tartaric acid-sodium hydroxide mixture, etc.), fumarate buffers (e.g. fumaric acid-monosodium fumarate mixture, fumaric acid-disodium fumarate mixture, monosodium fumarate-disodium fumarate mixture, etc.), gluconate buffers (e.g. gluconic acid-sodium gluconate mixture, gluconic acid-sodium hydroxide mixture, gluconic acid-potassium gluconate mixture, etc.), oxalate buffer (e.g. oxalic acid-sodium oxalate mixture, oxalic acid-sodium hydroxide mixture, oxalic acid-potassium oxalate mixture, etc), lactate buffers (e.g. lactic acid-sodium lactate mixture, lactic acid-sodium hydroxide mixture, lactic acid-potassium lactate mixture, etc.) and acetate buffers (e.g. acetic acid-sodium acetate mixture, acetic acid-sodium hydroxide mixture). Additionally, phosphate buffers, histidine buffers and trimethylamine salts, such as Tris can be used.

Preservatives can be added to retard microbial growth, and can be added in amounts ranging from 0.2%-1% (w/v). Suitable preservatives include phenol, benzyl alcohol, meta-cresol, methyl paraben, propyl paraben, octadecyldimethylbenzyl ammonium chloride, benzalconium halides (e.g. chloride, bromide, and iodide), hexamethonium chloride, and alkyl parabens, such as methyl or propyl paraben, catechol, resorcinol, cyclohexanol, and 3-pentanol. Isotonicifiers sometimes known as “stabilizers” can be added to ensure isotonicity of liquid compositions and include polhydric sugar alcohols, preferably trihydric or higher sugar alcohols, such as glycerin, erythritol, arabitol, xylitol, sorbitol and mannitol. Stabilizers refer to a broad category of excipients, which can range in function from a bulking agent to an additive, which solubilizes the therapeutic agent or helps to prevent denaturation or adherence to the container wall. Typical stabilizers can be polyhydric sugar alcohols (enumerated above); amino acids, such as arginine, lysine, glycine, glutamine, asparagine, histidine, alanine, ornithine, L-leucine, 2-phenylalanine, glutamic acid, threonine, etc., organic sugars or sugar alcohols, such as lactose, trehalose, stachyose, mannitol, sorbitol, xylitol, ribitol, myoinisitol, galactitol, glycerol and the like, including cyclitols, such as inositol; polyethylene glycol; amino acid polymers; sulfur containing reducing agents, such as urea, glutathione, thioctic acid, sodium thioglycolate, thioglycerol, α-monothioglycerol and sodium thio sulfate; low molecular weight polypeptides (e.g. peptides of 10 residues or fewer); proteins, such as human serum albumin, bovine serum albumin, gelatin or immunoglobulins; hydrophylic polymers, such as polyvinylpyrrolidone monosaccharides, such as xylose, mannose, fructose, glucose; disaccharides, such as lactose, maltose, sucrose and trisaccacharides, such as raffinose; and polysaccharides, such as dextran. Stabilizers can be present in the range from 0.1 to 10,000 weights per part of weight active protein.

Non-ionic surfactants or detergents (also known as “wetting agents”) can be added to help solubilize the therapeutic agent as well as to protect the therapeutic protein against agitation-induced aggregation, which also permits the formulation to be exposed to shear surface stressed without causing denaturation of the protein. Suitable non-ionic surfactants include polysorbates (20, 80, etc.), polyoxamers (184, 188, etc.), pluronic polyols, polyoxyethylene sorbitan monoethers (TWEEN®-20, TWEEN®-80, etc.). Non-ionic surfactants can be present in a range of about 0.05 mg/ml to about 1.0 mg/ml, or in a range of about 0.07 mg/ml to about 0.2 mg/ml.

Additional miscellaneous excipients include bulking agents (e.g. starch), chelating agents (e.g. EDTA), antioxidants (e.g. ascorbic acid, methionine, vitamin E), and co-solvents.

The formulation herein can also contain a second therapeutic agent in addition to an antibody or another inhibitor of the invention. Examples of suitable second therapeutic agents are provided below.

The dosing schedule can vary from once a month to daily depending on a number of clinical factors, including the type of disease, severity of disease, and the patient's sensitivity to the antibody or another inhibitor of the invention. In specific embodiments, an antibody or another inhibitor of the invention, is administered daily, twice weekly, three times a week, every 5 days, every 10 days, every two weeks, every three weeks, every four weeks or once a month, or in any range between any two of the foregoing values, for example from every four days to every month, from every 10 days to every two weeks, or from two to three times a week, etc.

The dosage of an antibody or another inhibitor of the invention, to be administered will vary according to the particular antibody, the subject, and the nature and severity of the disease, the physical condition of the subject, the therapeutic regimen (e.g. whether a second therapeutic agent is used), and the selected route of administration; the appropriate dosage can be readily determined by a person skilled in the art.

It will be recognized by one of skill in the art that the optimal quantity and spacing of individual dosages of an antibody or another inhibitor of the invention, will be determined by the nature and extent of the condition being treated, the form, route and site of administration, and the age and condition of the particular subject being treated, and that a physician will ultimately determine appropriate dosages to be used. This dosage can be repeated as often as appropriate. If side effects develop, the amount and/or frequency of the dosage can be altered or reduced, in accordance with normal clinical practice.

### Combination Therapy

In certain embodiments, the patient being treated in accordance with the invention, e.g. by an antibody of the invention, is also treated with conventional coagulants. For example, a patient suffering from excessive bleeding is typically also being treated with an anti-fibrinolytic agent, a platelet concentrate, a coagulation factor concentrate and/or fresh frozen plasma.

### Further Aspects

Yet another aspect of the invention is the use of an inhibitor (preferably an antibody) as defined hereinabove for promoting haemostasis.

Yet another aspect of the invention is a compound (preferably an antibody) as defined hereinabove for use in reducing the bleeding time in a patient suffering from excessive bleeding.

The invention further relates to a method of reducing the bleeding time, comprising administering to a subject an effective amount of an inhibitor (preferably an antibody) as defined hereinabove.

A further aspect of this invention is a method of treating a haemorrhagic condition, comprising administering to a patient in need thereof an effective amount of an inhibitor (preferably an antibody) as defined hereinabove. The haemorrhagic condition is preferably one of the conditions described above.

A further aspect of this invention is a method of preventing a haemorrhagic condition, comprising administering to a patient in need thereof an effective amount of an inhibitor (preferably an antibody) as defined hereinabove. The haemorrhagic condition is preferably one of the conditions described above.

### Further Discussion

Among others, and without intending to be bound by theory, the present inventors delineate an unexpected spatio-temporal control mechanism of thrombin activity that is platelet orchestrated and locally limits excessive fibrin formation after initial haemostatic platelet deposition. During platelet activation, the abundant platelet glycoprotein (GP) V is cleaved by thrombin. The present inventors e.g. demonstrated that thrombin-mediated shedding of GPV does not primarily regulate platelet activation in thrombus formation, but rather has a distinct function after platelet deposition and specifically limits thrombin-dependent generation of fibrin, a crucial mediator of vascular thrombo-inflammation. The spatio-temporal control of thrombin-dependent fibrin generation is also considered a potential therapeutic target to improve haemostasis.

The present inventors further delineate the function of platelet GPV that is proteolytically released by thrombin in the context of platelet activation at sites of vascular injury. Genetic blockade of thrombin-mediated shedding of GPV uncovered the crucial role of sGPV as a regulator of fibrin formation and thrombus growth. By localizing to the growing thrombus, sGPV restricts thrombin's function in thrombosis, and—as demonstrated by pharmacological application of rhGPV—provides protection from thrombo-inflammatory neurological damage in an experimental model of ischaemic stroke without causing haemostatic impairments. Conversely, specific blockade of thrombin-mediated GPV shedding can enhance local fibrin formation in a variety of contexts associated with severe defects in platelet function. This unique spatio-temporal control of thrombin activity by GPV can thus be harnessed to promote haemostasis.

Accordingly, in view of the goals described above, and akin to strategies that inhibit the inhibitors' of coagulation, the present inventors propose a therapeutic strategy of tailored activation of haemostatic fibrin plug formation in the spatio-temporal context of platelet deposition at sites of vessel wall injury. By increasing local thrombin bioavailability without compromising scavenging of thrombin by endothelial cell-expressed thrombomodulin, this approach has little risk to interfere with physiological anticoagulation in the body and vascular protective and anti-inflammatory signalling of the protein C-PAR1 pathway¹⁰.

### Overview of Nucleotide and Amino Acid Sequences

An overview of nucleotide and amino acid sequences is provided in the following Table 1, where “LUM/B” and “LUM11” designate particularly preferred antibodies of the present invention, “HC” refers to “heavy chain” and “LC” refers to “light chain” Table 1:

| SEQ ID NO: | Type of Sequence | Sequence/Comments |
|---|---|---|
| 1 | LUM/B HC CDR1 | DYNMA |
| 2 | LUM/B HC CDR2 | TFIYDGSNTYYGDSVKG |
| 3 | LUM/B HC CDR3 | HTWANWFAY |
| 4 | LUM/B LC CDR1 | RGSQNINNYLA |
| 5 | LUM/B LC CDR2 | STNSLQT |
| 6 | LUM/B LC CDR3 | YQYNNGYT |
| 7 | LUM/B HC AA | LUM/B heavy chain amino acid sequence |
| 8 | LUM/B HC AA sig | LUM/B heavy chain amino acid sequence with signal |
| | | sequence |
| 9 | LUM/B LC AA | LUM/B light chain amino acid sequence |
| 10 | LUM/B LC AA sig | LUM/B light chain amino acid sequence with signal |
| | | sequence |
| 11 | LUM/B HC NU | LUM/B heavy chain nucleic acid sequence |
| 12 | LUM/B HC NU sig | LUM/B heavy chain nucleic acid sequence with signal |
| | | sequence |
| 13 | LUM/B LC NU | LUM/B light chain nucleic acid sequence |
| 14 | LUM/B LC NU sig | LUM/B light chain amino acid sequence with signal |
| | | sequence |
| 15 | LUM11 HC CDR1 | SYGVN |
| 16 | LUM11 HC CDR2 | AISSGGSTYYNSSLKS |
| 17 | LUM11 HC CDR3 | GYYGYDYDYFDY |
| 18 | LUM11 LC CDR1 | KGSQNINNYLA |
| 19 | LUM11 LC CDR2 | NTNSLQT |
| 20 | LUM11 LC CDR3 | YQYNNGYT |
| 21 | LUM11 HC AA | LUM11 heavy chain amino acid sequence |
| 22 | LUM11 HC AA sig | LUM11 heavy chain amino acid sequence with signal |
| | | sequence |
| 23 | LUM11 LC AA | LUM11 light chain amino acid sequence |
| 24 | LUM11 LC AA sig | LUM11 light chain amino acid sequence with signal |
| | | sequence |
| 25 | LUM11 HC NU | LUM11 heavy chain nucleic acid sequence |
| 26 | LUM11 HC NU sig | LUM11 heavy chain nucleic acid sequence with signal |
| | | sequence |
| 27 | LUM11 LC NU | LUM11 light chain nucleic acid sequence |
| 28 | LUM11 LC NU | LUM11 light chain amino acid sequence with signal |
| | | sequence |
| 29 | human GPV NU | nucleic acid sequence encoding human GPV |
| 30 | human GPV AA sig | human GPV amino acid sequence with signal sequence |
| 31 | human GPV AA | human GPV amino acid sequence |
| 32 | murine GPV NU | nucleic acid sequence encoding murine GPV |
| 33 | murine GPV AA sig | human GPV amino acid sequence with signal sequence |
| 34 | murine GPV AA | murine GPV amino acid sequence |

### EXAMPLES

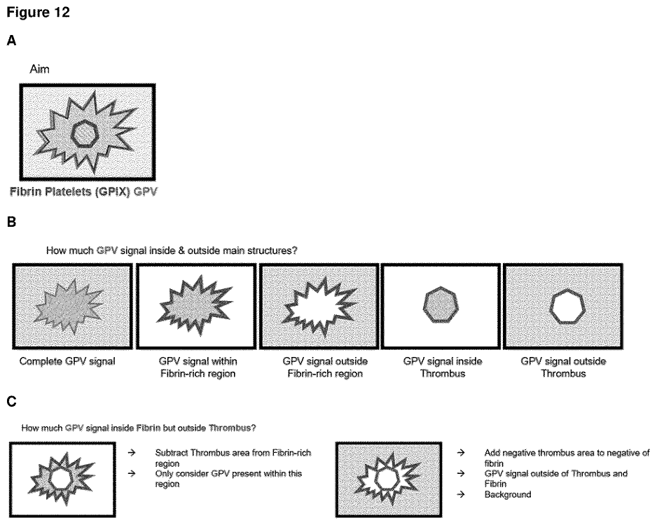

### Example 1.1—Methods for Experiments for FIGS. 1 to 5 and 8 to 17

### Animals

Mice were maintained under specific pathogen-free conditions (constant temperature of 20-24° C. and 45-65% humidity with a 12-h light-dark cycle, ad libitum water and food access) and experiments were performed in accordance with German law and the governmental bodies, and with approval from the District of Lower Franconia.

Gp5^{-/- 12} and Itga2^{-/- 49} mice were kindly provided by Frangois Lanza (Inserm-Université de Strasbourg, Strasbourg, France) and Beate Eckes (Department of Dermatology, University of Cologne, Cologne, Germany), respectively. Gp5^{dThr} mice, which carry a point mutation in the thrombin cleavage site of GPV, were generated are described in Supplemental FIG. 1A). Gp5^{dThr} mice were intercrossed with Flip-positive mice to delete the Neo-cassette and backcrossed to C57Bl/6J background. RhoA^{fl/fl 50} mice were kindly provided by Cord Brakebusch (University of Copenhagen, Copenhagen, Denmark). To generate MK-/platelet-specific knockout mice, the floxed mice were intercrossed with mice carrying the Cre recombinase under control of the Pf4 (platelet factor) promoter. Nbeal2^{-/-43} mice were described previously. All mice were kept on a C57Bl/6J background and all animal experiments and the analysis of the corresponding data were performed blinded.

### Human Blood Samples

For this study, blood samples were obtained from healthy volunteers, free from anticoagulant or anti-platelet therapy for at least 4 weeks, following written informed consent in accordance with the Declaration of Helsinki and after approval by the Institutional Review Boards (IRB) of University of Wurzburg.

### Chemicals

Midazolam (Roche Pharma AG), dorbene (Pfizer) and fentanyl (Janssen-Cilag GmbH) were used according to regulations of the local authorities. Clopidogrel was from Sanofi, low-molecular-weight heparin from ratiopharm GmbH and recombinant hirudin from Coachrom. Human fibrinogen (#F4883), bovine thrombin (T4648), N-ethylmaleimide (NEM, 23030) and Prostacyclin (PGI₂) were from Sigma-Aldrich. Prolong Glass antifade mountant (P36980) and fibrin(ogen) AF488 (F13191) were from Thermo Scientific. Iron(III)chloride (FeCl₃) was from Roth. A23187 was from AppliChem, ionomycin from VWR. Convulxin was from Axxora. Fibrillar type I collagen (Horm) was from Takeda, Rhodocytin was provided by Johannes Eble (University of Munster, Munster, Germany). Collagen-related peptide (CRP) was generated as previously described⁵¹. Integrilin was from GlaxoSmithKline. Human thrombin was from Roche (Sigma-Aldrich #10602400001). Apyrase type III was from GE Healthcare, biotinylated thrombin (69672-3) was from Merck/Millipore. Z-GGR-AMC-HCl was from Bachem, thrombin calibrator was from Stago, human recombinant tissue factor (Dade Innovin) was from Siemens Healthcare. Fluorogenic thrombin substrate Pefafluor TH was from Pentapharm. Donkey anti-rat IgG FITC (#112095068) was from Jackson Immuno Research (West Grove, PA, USA). DNA aptamers (HD1: GGTTGGTGTGGTTGG, HD22: AGTCCGTGGTAGGGCAGGTTGGGGTGACT) to block thrombin exosites and control aptamer HD23 (AGTCCGTAATAAAGCAGGTTAAAAT GACT) were from Future Synthesis (Poznan, Poland).

### Antibodies

PAC-1-FITC (#340507) and anti-CD62P-APC (#550888) antibodies were from BD Biosciences, control rat IgG (#14131) was from Sigma. Anti-mGPV antibody (#AF6990) for Western blot analysis was from R&D. The platelet-depletion antibody R300 (rat anti-GPIbα IgG antibody) was from Emfret Analytics (Eibelstadt, Germany).

Table 2. In house generated antibodies

| | | | |
|---|---|---|---|
| Mouse P-selectin | WUG1.9 | In-house generated | 52 |
| Mouse GPllb/Illa | JON/A-PE | In-house generated | 53 |
| Mouse GPIX | p0p6 | In-house generated | 54 |
| Mouse GPV | DOM/B | In-house generated | this study |
| Mouse GPV | DOM/C | In-house generated | this study |
| Mouse GPV | DOM3 | In-house generated | 55 |
| Human GPV | LUM/B | In-house generated | this study |
| Human GPV | LUM1 | In-house generated | this study |
| Human GPV | LUM2 | In-house generated | this study |
| Human GPV | LUM3 | In-house generated | this study |
| Human GPV | LUM4 | In-house generated | this study |
| Human GPV | LUM5 | In-house generated | this study |
| Mouse α2 integrin | LEN/B | In-house generated | 56 |
| Mouse GPlbα | p0p/B | In-house generated | 28 |
| Mouse/human GPlbß | p0p1 | In-house generated | 57 |

The novel antibodies were generated by hybridoma technology following immunisation of Gp5^{-/-} mice or Wistar rats with recombinant hGPV protein or GPV immunoprecipitated from mouse or human platelet lysates.

Expression and Purification of Recombinant hGPV

The gene fragment encoding the GP64 signal peptide (MVSAIVLYVLLAAAAHSAFA), human GPV extracellular domain (aa 17-518), and a decahistidine tag was amplified, inserted into pFastBac™ dual vector and transformed into DH10Bac E. coli strain (ThermoFisher Scientific). The resulting bacmid DNA was prepared and then transfected into Sf9 insect cells using cellFectin II reagent (ThermoFisher). The high-titer P2 baculovirus stock was prepared from scaled-up Sf9 cells in Sf-900 II serum-free media following the instruction of Bac-to-Bac™ Baculovirus Expression System (ThermoFisher Scientific) and used to induce hGPV expression in Sf9 cells (2×10⁶ cells/ml, MOI 2) for 72 hours. hGPV was purified from insect cell media by Ni-affinity chromatography using a GE Healthcare Ni Sepharose excel column (elution buffer: 20 mM sodium phosphate, 500 mM NaCl, 500 mM imidazole, pH 7.4), followed by size exclusion chromatography using a GE Healthcare HiLoad 16/600 Superdex 200 μg column (elution buffer: phosphate buffered saline (PBS) containing 0.1% tween20, pH 7.4). The purified protein was stored at −80° C. in PBS containing 0.1% tween20 and 20% glycerol.

### Antibody Treatment

For the antibody treatment experiments the mice were separated into antibody and control treatment group in a randomized manner using https://www.random.ora/iists/. 100 μg JAQ1 IgG were injected intraperitoneally at day 7 and day 5 prior to the experiment, resulting in a GPVI knockout like phenotype³¹. All other antibodies (each 100 μg) were injected iv. or i.p. directly before the experiment.

### Platelet Depletion

Thrombocytopenia was induced by intravenous injection of rat anti-GPIbα IgG antibody R300 (Emfret Analytics, Eibelstadt, Germany, 0.14-0.18 μg/g body weight). This low dose of platelet depletion antibody reduced the platelet count to 5-10% of the initial platelet count⁴⁶. Peripheral platelet count was determined by flow cytometry 16 h after platelet depletion (prior to tail bleeding time experiment).

Treatment with Clopidogrel or rhGPV

Mice were fed orally with 3 mg/kg clopidogrel 48 h and 24 h before the experiment. Mice were injected intravenously with 20 μg rhGPV 5 min before the experiment.

### Preparation of PRP and Washed Platelets

Mice were anesthetised using isoflurane and bled to 300 μl heparin (20 U/ml in TBS, pH 7.3, Ratiopharm). The blood was centrifuged twice at 300 g for 6 min to obtain platelet-rich plasma (PRP). PRP was supplemented with 0.02 U/ml apyrase (A610, Sigma-Aldrich) and 0.1 μg/ml PGI₂ (P6188, Sigma-Aldrich) and platelets were pelleted by centrifugation at 800 g for 5 min, washed twice with Tyrode's buffer (134 mM NaCl, 0.34 mM Na₂HPO₄, 2.9 mM KCl, 12 mM NaHCO₃, 5 mM HEPES, 5 mM glucose, 0.35% BSA, pH7.4) containing 0.02 U/ml apyrase and 0.1 μg/ml PGI₂. The platelets were allowed to rest for at least 30 min at 37° C. prior to experiments.

### Aggregation Assay

Washed platelets (160 μl with 1.5×10⁵ platelets/μl) and PRP (only used for ADP stimulation) were prepared as described. For aggregometry, washed platelets were analysed in the absence (thrombin) or presence (all other agonists) of 70 μg/ml human fibrinogen. Antibodies (10 μg/ml) were preincubated for 5 min at 37° C. prior to the experiment. Light transmission was recorded on a four-channel aggregometer (Fibrintimer; APACT, Hamburg, Germany) for 10 min or 20 min (in the presence of LEN/B) and expressed in arbitrary units, with buffer representing 100% light transmission. Platelet aggregation was induced by addition of the indicated agonists.

### Flow Cytometry

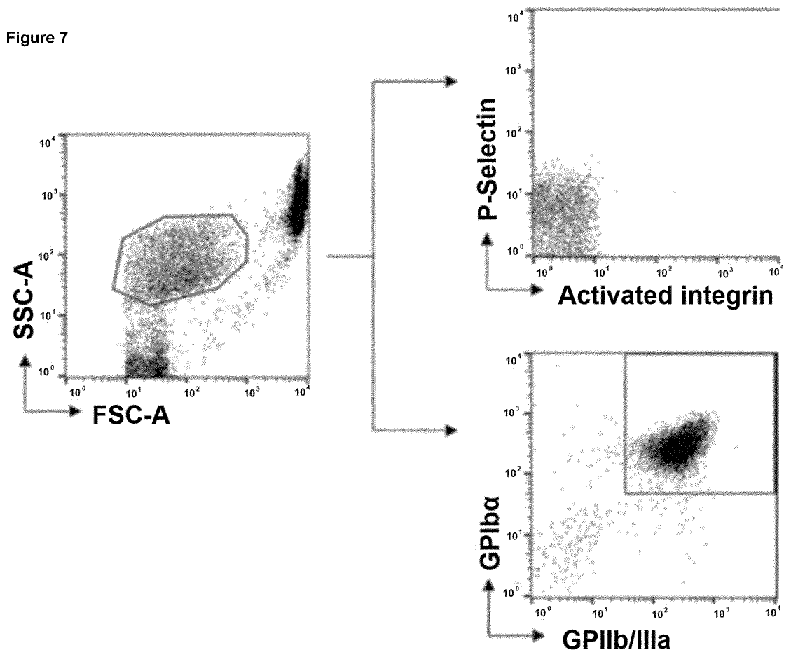

To determine glycoprotein surface expression levels, whole blood was diluted 1:20 with Ca²⁺-free Tyrode's buffer or PBS and stained with saturating amounts of fluorophore-conjugated antibodies for 15 min at RT in the dark. Washed platelets adjusted to 50000/μl in Tyrode's buffer with Ca²⁺ were stimulated with the indicated agonists and incubated with saturating amounts of fluorophore-conjugated antibodies to determine platelet activation or thrombin-mediated cleavage of GPV. Human Thrombin (Sigma #10602400001) was used to stimulate platelets. All samples were analysed directly after addition of 500 μl PBS on a FACSCalibur (BD Biosciences, Heidelberg, Germany). Exemplified gating strategy based on FSC/SSC characteristics are shown in FIG. 7.

### Thrombin- and NEM-Induced Cleavage of GPV

Washed platelets were adjusted to a concentration of 1×10⁶ platelets/μl in Tyrode's buffer without Ca²⁺ and diluted 1:1 with Tyrode's buffer without Ca²⁺ (for resting and thrombin-stimulated samples (human Thrombin (Sigma #10602400001)) and with Tyrode's buffer with Ca²⁺ (for NEM (2 mM f.c.)-incubated samples). Stimulation with 867 μM thrombin (human thrombin: 0.1 U/ml is equivalent for 867 μM thrombin) for 30 min at 37° C. was performed in the presence of 40 μg/ml integrilin and 5 μM EGTA to prevent platelet aggregation. Afterwards, platelet suspension was diluted and incubated with saturating amounts of FITC-conjugated platelet surface antibodies and were directly analysed on a FACSCalibur. The residual platelet suspension was pelleted, and the supernatant analysed in a GPV ELISA.

### GPV ELISA

96-well plates (Hartenstein, Wurzburg, Germany, F-Form) were coated with 50 μl/well DOM/C antibody (30 μg/ml) in carbonate buffer o/n at 4° C., blocked with 5% non-fat dried milk in PBS for 2 h at 37° C. and washed. Samples were applied to plates, incubated for 1 h at 37° C. and washed. Plates were incubated with HRP-labeled DOM/B antibody for 1 h, washed again 3 times and developed using TMB substrate. The reaction was stopped by addition of 0.5 M H₂SO₄. Optical density was measured on a Multiskan Ex device (Thermo Electron Corporation, Braunschweig, Germany). Absorbance was read at 450 nm, the 620 nm filter served as reference wavelength. Plasma samples from Gp5^{-/-} mice served as negative control, supernatant after platelet thrombin stimulation as positive control.

### Clot Retraction

Clot retraction studies were performed at 37° C. in an aggregometer tube containing diluted PRP (3×10⁵ platelets/μl), thrombin (34.68 nM), and CaCl₂ (20 mM). Clot retraction was recorded with a camera over a time span of 2 h after activation.

### Thrombin Time

To determine thrombin time, citrated PRP was diluted 1:1 in PBS and stimulated with 17.5 nM f.c. bovine thrombin (equivalent to 2 U/ml). Thrombin time was analysed with a 4-channel mechanical ball coagulometer (Merlin medical, Lemgo, Germany).

### Thrombin Generation

Thrombin generation was quantified in recalcified citrate-anticoagulated PRP with platelet count adjusted to 1.5×10⁵ platelets/μl. Platelets were resuspendend in pooled plasma preparations from 2-4 mice with the same genotype. Platelets were activated with the indicated agonists for 15 min at 37° C. After stimulation, samples in duplicates (4 vol) were transferred to a polystyrene 96-Immulon 2HB well plate already containing 1 vol of thrombin calibrator or tissue factor (1 μM f.c.). Coagulation was started by adding 1 vol of fluorescent thrombin substrate (2.5 mM Z-GGR-AMC). Thrombin generation was measured as described previously^{58,59} and analysed using the Thrombinoscope™ software (version 5.0.0.742, ThrombinoscopeBV, Maastricht, The Netherlands).

### Static Fibrin Polymerisation

Unlabelled fibrinogen (1.35 mg/ml f.c.) and Alexa Fluor A488-labelled fibrinogen (45 μg/ml f.c.) were mixed (30:1) in the absence or presence of rhGPV (20 μg/ml, stained with LUM/B AF647). Fibrin polymerisation was initiated by addition of 867 μM thrombin or 1 U/ml batroxobin (Loxo, Dossenheim, Germany) in the presence of 5 mM CaCl₂. The mixture was immediately transferred to an uncoated 15μ-slide 8-well (Ibidi GmbH, Gräfelfing, Germany), and placed in a dark humidity chamber for 2 h at room temperature to allow fibrin polymerisation. Images were obtained using a Leica SP8 inverted microscope with a 63× oil immersion lens. Optical z-stacks (8 μm, 0.1 step size, Nyquist conform) were deconvolved (Huygens Essential Software) and are shown as maximum projection (Image J software).

### Coagulation Flow Chamber

Glass coverslips were coated with collagen type I (10 μl, 50 μg/ml) and tissue factor (TF; 10 μl, 100 μM or 10 μM for experiments with human blood or mouse blood, respectively) and blocked with 1% BSA/PBS. Citrated whole blood was recalcified by co-infusion with 6.3 mM CaCl₂ (f.c.) and 3.2 mM MgCl₂ (f.c.) and perfused over the collagen/TF spots for up to 6 min at a shear rate of 1000 s^{-1 60} Before each experiment, blood samples were pre-labeled with Alexa Fluor™ (AF) 488-conjugated fibrinogen, an anti-GPIX derivative (mouse) or anti-GPIbβ derivative (human) to stain platelets. For human samples, control and antibody-treated samples of the same donor were always run in parallel.

### 1. Time Series Experiments:

Before each experiment, blood samples were preincubated with AF488-conjugated fibrinogen, AF647-conjugated anti-GPIX derivative (mouse) or AF647-conjugated anti-GPIbβ derivative (human) platelets. For time series experiments, fluorescence microscopic images were captured at 30 s intervals, to evaluate kinetics for up to 6 min (Leica DMI 6000 B, 63× objective). Recorded images were further processed with the background subtraction method ICC (Instant Computational Clearing) to remove out-of-focus blur on a Leica Thunder microscope. Next, the exported images were analysed for surface area coverage of fibrin formation with self-written Python scripts⁶¹. In detail, in the first step an entropy filter with a disk size of 5 pixel was applied, followed by a median filter (disk size 10 pixel) and Otsu-thresholding. To compensate for a nearly empty field of view during the first images in the time series, the present inventors introduced a “scaling factor” sf with which the present inventors multiplied the found Otsu-threshold value, and thus increased the threshold slightly for the first images (usually the first 60-150 s, with sf=1.03-1.05). The thresholded area (as fraction of the whole field of view) represents the area covered by fibrin.

For heatmap representation, mean values were univariate scaled from −4 to 4. Gene effect heatmaps were constructed by subtracting scaled average values of the control strain from the mutant strain. For details see³⁵.

### 2. 3D Confocal Microscopy

Samples were stained as described for 1. Time series experiments. To acquire z-stacks of complete thrombi, samples were fixed with 4% PFA/PBS, mounted with Prolong Glass (Thermo Scientific) and further analysed by confocal microscopy (Leica SP8 inverted microscope). Z-stack of thrombi were acquired at a SP8 confocal microscope (Leica) Hyvolution mode, 63×, z-step: 0.1 μm. Images were deconvolved using Huygens Professional Software (v 21.04) with a signal-to-noise ratio (SNR) between 2-10 (identical for regions of interest in the same animal), an automatic background subtraction using the in/near object option with a search radius of 2 μm, and maximal 40 iterations. The deconvolved data set was exported as Imaris file format and visualised with Fiji⁶² (https://www.biovoxxel.de/development/).

To analyse GPV-fibrin localisation outside the thrombus, blood samples were preincubated with AF488-conjugated fibrinogen, AF546-conjugated anti-GPV derivative and AF405-conjugated anti-GPIX derivative. Single images from the bottom of a thrombus were acquired using a Zeiss LSM 980 Airyscan microscope (63× objective) in superresolution mode using the smart setup. Images were deconvolved by Zeiss ZEN software and analysed with Fiji. First, masks from the fibrin(ogen) and platelet (GPIX) channel were generated using Li thresholding. Masks were then used as “positive” (pixel intensity=1 inside structure and =0 outside the structure) and “negative” imprints (pixel intensity=0 inside the structure and =1 outside the structure) and applied to the GPV channel. To analyse GPV intensities outside thrombus but inside fibrin fibres, the positive fibrin mask is multiplied with the negative thrombus mask.

In detail, after Li thresholding the intensity values in the binarized images were changed to 0 and 1 by dividing through 255. Next, this procedure for preparation of the positive and negative binary mask was repeated for the GPIX channel. Afterwards, the prepared masks based on single-cannel thresholds were combined by multiplication (leading to a value of 1, if positive in both masks). This resulted in the following regions:
Inside fibrin but non-platelet area
Outside fibrin & non-platelet area=>background count rate

Finally, the intensity of GPV was determined in the area of fibrin by multiplication of the obtained masks with GPV channel. Values outside the mask were set to 0, pixel inside the mask have an intensity of 1, thus the original intensity of GPV was preserved. This calculation was performed for all masks and surface coverage as well as raw integrated density were determined. Then, the average intensity per pixel was calculated inside the covered area and the raw integrated density was divided by the number of pixels to obtain the average intensity per non-zero pixel.

For each step, a Fiji macro was recorded. All Fiji macros and Python scripts used for the fluorescence image analysis can be downloaded from https:\\github.com\HeinzeLab\GPV-flowchamber.

### Thrombin Activity

Coagulation flow assay was performed as described above without staining for platelets and fibrin(ogen). The outflow was collected in 10 mM EDTA and 1.5 μM HD1. Thrombin activity was measured immediately using the fluorogenic thrombin substrate Pefafluor TH (Pentapharm) at 460 nm.

Thrombin activity in the formed thrombi was determined using the fluorogenic thrombin substrate Z-GGR-AMC.

### Western Blot After Pulldown

Washed platelets were adjusted to 1×10⁶ platelets/μl and either left unstimulated or were stimulated with biotinylated thrombin (433 μM) for 15 min at 37° C. Where indicated, Hirudin (0.1 U/ml) or GM6001 (100 μM f.c.) were added before platelet stimulation. Platelets were pelleted and the supernatant was incubated with magnetic Streptavidin beads to pulldown biotinylated thrombin. After incubation, beads were collected and washed. The eluate was used for Western blot analysis and the samples were detected with an anti-GPV antibody (R&D).

### Tail Bleeding Time

Mice were anaesthetised by intraperitoneal injection of triple anaesthesia (Dormitor 0.5 μg/g, Midazolam 5 μg/g, and Fentanyl 0.05 μg/g body weight) and a 1-mm segment of the tail tip was removed using a scalpel. Tail bleeding was monitored by gently absorbing blood on filter paper at 20 s intervals without directly contacting the wound site. When no blood was observed on the paper, bleeding was determined to have ceased. The experiment was manually stopped after 20 min by cauterization.

### Light Sheet Fluorescence Microscopy (LSFM)

### Sample Preparation

The vasculature was stained by intravenous injection of AF647-conjugated anti-CD105 (clone MJ7/19, purified in-house, 0.4 μg⁻¹) and AF647-conjugated anti-CD31 (BioLegend, clone 390, 0.4 μg g⁻¹). 30 min after in vivo labeling mice were anesthetized by intraperitoneal injection of medetomidine 0.5 μg/g, midazolam 5 μg/g and fentanyl 0.05 μg/g body weight and transcardially perfused with ice-cold PBS to wash out the blood and ice-cold 4% paraformaldehyde (PFA, P6148, Sigma-Aldrich, Germany, pH 7.2). Brains were removed, dehydrated in methanol solutions of increasing concentrations (50%, 70%, 95%, 100%) to fix the tissue. Brains were then harvested and stored in 4% PFA for 30 min. Samples were then washed in PBS, followed by dehydration in a graded methanol (Sigma-Aldrich) series (50%, 70%, 95%, 100% for 30 min each) at RT and stored at 4° C. overnight. The methanol was replaced stepwise by a clearing solution consisting of one part benzyl alcohol to two parts benzyl benzoate (BABB, catalog nos. 305197 and B6630, Sigma-Aldrich). After incubation in the clearing solution for at least 2 h at RT, tissue specimens became optically transparent and were used for LSFM imaging on the following day.

Optically cleared brains were imaged with a custom-build light sheet microscope equipped with two EC Epiplan. Neofluar 2.5×/0.06 M27 excitation objectives (Zeiss, Germany) and a HCX FLUOTAR 5×/0.15 Dry detection objective (Leica, Germany) as previously described²⁹ with a voxel size of 2.6 μm and a z-spacing of 5 μm (Pixel size: 2.6×2.6×5 μm). Major parts of the LSFM have been described previously⁶³. Additionally, to the fluorescence signal of the AF647-conjugated antibodies staining the vessel system also the brain autofluorescence was collected by excitation at 488 nm/emission 520 nm.

### Segmentation of Brain LSFM Images

Images acquired by LSFM were saved in TIFF format and converted to the Imaris file format (Imaris 9.9, Bitplane, Oxford) for further processing and segmentation. Using the built-in image processing tools, first the background was subtracted from both channels and secondly a 3×3×3 voxel median filter was applied to the vessel channel (AF647 fluorescence). Next, the median filtered vessel channel was segmented using the surface tool. Here, a four-voxel smoothing (10.4 μm) and a local contrast intensity thresholding (10 μm diameter) was applied. The intensity threshold was adjusted manually to ~50% of the automatically proposed value. Finally, objects smaller than 1000 voxel were removed.

### Determination of Vessel Diameter

To determine the diameter of the vessel at selected regions of interest, the generated vessel surface was masked onto the vessel fluorescence such that the intensity outside the surface was zero while inside the surface the original, median filtered intensity values are present.

To estimate the diameter, the measurement points option in Imaris was used, which were placed directly on the border of the selected vessel regions of interest. Correct placing of the measurement points was ensured by 3D inspection of the images.

Transient Middle Cerebral Artery Occlusion (tMCAO)

Focal cerebral ischaemia was induced by a transient MCA occlusion (tMCAO) as described⁶⁴. Briefly, a silicon-coated thread was advanced through the carotid artery up to the origin of the MCA causing an MCA infarction. After an occlusion time of 60 min, the filament was removed allowing reperfusion of the MCA territory. The extent of oedema corrected brain infarction was quantitatively assessed 24 h after reperfusion on 2,3,5-triphenyltetrazolium chloride-stained consecutive brain sections. Neurological function was analyzed calculating a neuroscore (score 0-10) based on the direct sum of the Grip test (score 0-5) and the inverted Bederson score (score 0-5).

### PcomA Scores

PcomA scores (posterior communicating artery) were determined in brains from mice that were perfused with PBS followed 3 ml black ink diluted in 4% PFA (1:5 v/v).

### Mechanical Injury of the Abdominal Aorta

To open the abdominal cavity of anaesthetised mice (10 to 16-weeks old), a longitudinal midline incision was performed, and the abdominal aorta was exposed. A Doppler ultrasonic flow probe (0.5PSB699, Transonic Systems, Maastricht, The Netherlands) was placed around the vessel and thrombus formation was induced by a single firm compression (20 s) with a forceps upstream of the flow probe. Blood flow was monitored over 30 min or until complete occlusion occurred (blood flow stopped for >5 min). The abdominal aorta was excised and embedded in Tissue Tek. Sections (5 μm) were fixed and stained according to Carstairs method to distinguish platelets and fibrin⁶⁵.

### FeCl₃-Induced Injury of Mesenteric Arterioles

3 to 4-weeks old mice were anaesthetised, and the mesentery was exteriorised. Arterioles (35-60 μm diameter) were visualised with a Zeiss Axiovert 200 inverted microscope (10×/0.25 air objective) equipped with a 100-W HBO mercury lamp and a CooISNAP-EZ camera (Visitron, Munich, Germany). Endothelial injury was induced by topical application of a 3 mm² filter paper saturated with ferric chloride (FeCl₃; 20%). Adhesion and aggregation of fluorescently labeled platelets (Dylight 488-conjugated anti-GPIX derivative) was monitored for 40 min or until complete occlusion occurred (blood flow stopped for >1 min).

### Data Analysis

The presented results are mean±SD from three independent experiments per group and lines represent mean values, if not stated otherwise. Normal distribution was tested using the Shapiro-Wilk normality test. If passed, p-values were calculated using the two tailed unpaired t-test (2 groups), if the values were not normally distributed, differences between two groups were analysed using the Mann-Whitney two-tailed test. For more than two groups, one-way ANOVA (Kruskal-Wallis test) followed by Dunn's test for multiple comparisons was performed using GraphPad Prism software (V7.05.). Two tailed paired t-test (2 groups, normally distributed), Wilcoxon matched-pairs signed rank test (2 groups, not normally distributed) and Friedman test followed by Dunn's test for multiple comparisons (more than 2 groups, not normally distributed) were used for paired comparisons. For statistical analysis of non-occluded vs. occluded vessels, Fisher's exact t-test was used. P-values <0.05 were considered statistically significant.

### Example 1.2—Results of Experiments of Example 1.1

### Thrombus Formation is Accelerated in GPV Mutant Mice

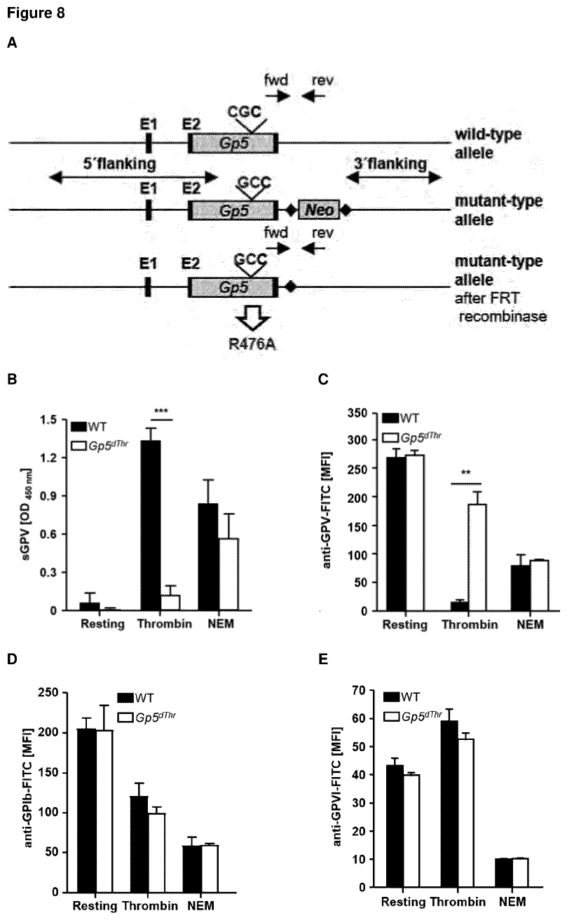

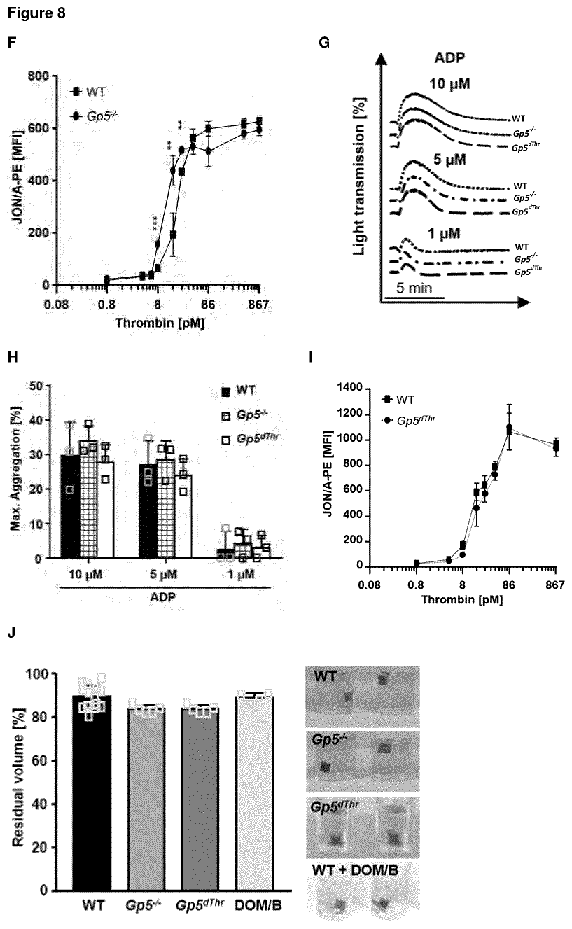

The present inventors studied the role of GPV in thrombus formation by comparing WT and Gp5^{-/-} mice in FeCl₃-induced thrombosis of mesenteric arterioles in vivo. In line with previous observations²⁰, Gp5^{-/-} mice displayed faster onset of thrombus formation and shortened occlusion times without increased embolization, indicating a prothrombotic phenotype in the absence of GPV (FIG. 1A, B). Platelet hyperreactivity to thrombin is the presumed but unproven mechanism for enhanced thrombosis in Gp5^{-/-} mice and thought to be related to thrombin-mediated cleavage of GPV. To directly study the relevance of thrombin-mediated GPV cleavage, the present inventors generated a mouse carrying a point mutation in the thrombin cleavage site of GPV (Gp5^{dThr}; FIG. 8A). Platelets of these mice showed unaltered surface expression levels of GPV compared to WT and GPV was completely resistant to cleavage by thrombin (FIG. 8B, C, Table 3). In contrast, cleavage of the mutant GPV by endogenous a disintegrin and metalloproteinase (ADAM)17²¹ was not affected (FIG. 8B-E), demonstrating the thrombin specificity of the Gp5^{dThr} mutation. Unexpectedly, Gp5^{dThr} mice displayed accelerated thrombus formation in the FeCl₃ arteriolar injury model, and in this respect resembled Gp5^{-/-} mice (FIG. 1C, D).

### Table 3

### Analysis of Platelet Count, Size, and Surface Expression of Glycoproteins in Gp5^{dThr} Mice.

Mean platelet count and size were determined using a Sysmex cell counter. Surface expression of platelet glycoproteins was determined by flow cytometry. Diluted whole blood was stained with FITC-labelled antibodies at saturating amounts for 15 min at RT. Platelets were analysed immediately on a FACSCalibur. Results are expressed as mean fluorescence intensity (MFI)±SD. n=4-6, two-tailed unpaired t-test with Welch's correction, *p<0.05.

| | WT | Gp5dThr | P-value | Sig. |
|---|---|---|---|---|
| Platelet count | 85.8 ± 25.0 | 102.8 ± 20.4 | 0.246 | n.s. |
| [10³/ul] | | | | |
| MPV [fl] | 5.4 ± 0.2 | 5.5 ± 0.2 | 0.468 | n.s. |
| WBC [10/ul] | 76.7 ± 1.6 | 76.0 ± 15.2 | 0.954 | n.s. |
| RBC [104/ul] | 89.0 ± 25 | 93.2 ± 3.1 | 0.700 | n.s. |
| HGB [g/dl] | 1.4 ± 0.3 | 1.5 ± 0.1 | 0.430 | n.s. |
| HCT [%] | 4.8 ± 1.3 | 5.1 ± 0.2 | 0.522 | n.s. |
| GPlb [MFI] | 341.3 ± 20.3 | 354.3 ± 5.3 | 0.293 | n.s. |
| GPV [MFI] | 318.5 ± 11.9 | 289 ± 32.6 | 0.120 | n.s. |
| GPIX [MFI] | 308.75 ± 8.66 | 317.75 ± 4.79 | 0.132 | n.s. |
| GPVI [MFI] | 33.25 ± 0.96 | 34 ± 1.41 | 0.418 | n.s. |
| αllbß3 [MFI] | 338.75 ± 27.68 | 352 ± 12 | 0.428 | n.s. |
| ß3 [MFI] | 170.6 ± 16.1 | 153.2 ± 5.7 | 0.072 | n.s. |
| α5 [MFI] | 18.3 ± 0.5 | 18.3 ± 1.7 | 1 | n.s. |
| α2 [MFI] | 38.75 ± 0.957 | 39.25 ± 3.59 | 0.803 | n.s. |
| CD9 [MFI] | 669.75 ± 45.15 | 746 ± 2.83 | 0.043 | * |
| CLEC-2 [MFI] | 90.75 ± 6.24 | 82.5 ± 15.26 | 0.374 | n.s. |
| CD84 [MFI] | 25 ± 1.63 | 25.5 ± 0.58 | 0.597 | n.s. |

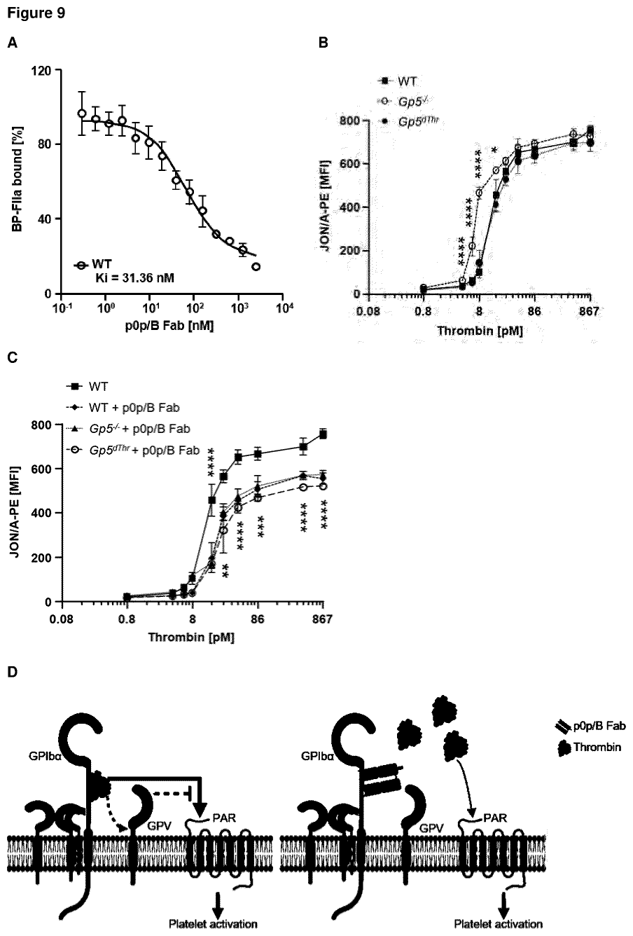

In a series of experiments, the present inventors addressed the possibility that excessive platelet activation also caused the prothrombotic phenotype of Gp5^{dThr} mice. Loss of surface GPV led to hyperreactivity of Gp5^{-/-} platelets specifically at lower thrombin concentrations but not with other agonists (FIG. 1E, G, H, FIG. 8F-H), as previously shown^{1520,22-24}. In sharp contrast to Gp5^{-/-} platelets, measurements of P-selectin exposure (FIG. 1F) and αIIbβ3 integrin activation and platelet aggregation (FIG. 81, 1G, H) showed that Gp5^{dThr} platelets were not hyperreactive at threshold thrombin concentration. Of note, both Gp5^{-/-} and Gp5^{dThr} PRP showed unaltered clot retraction (FIG. 8J). The present inventors next tested the hypothesis that membrane-bound GPV might act as a regulator of thrombin-mediated PAR activation²⁵ supported by the GPIbα high affinity binding of thrombin^{26,27}. Blockade of the GPIbα-thrombin interaction on mouse platelets with Fab-fragments of the anti-GPIbα antibody p0p/B^{28 29} (FIG. 9A) indeed diminished platelet activation, particularly at low thrombin concentrations (FIG. 9C). Although human and mouse platelets are activated by thrombin through different PARs, these antibody inhibition data indicated that mouse platelets are similar to human platelets²⁵ in requiring GPIbα for thrombin-induced activation at threshold agonist concentrations. Remarkably, the anti-GPIbα antibody completely abolished the enhanced activation of Gp5^{-/-} relative to WT platelets (FIG. 9C), implying that loss of GPV sensitised to GPIbα-dependent thrombin signalling. In contrast, activation of Gp5^{dThr} platelets at threshold concentrations of thrombin was indistinguishable from WT platelets with or without anti-GPIbα p0p/B (FIG. 9B, C). Thus, surface GPV regulates platelet responsiveness to thrombin primarily by interference with GPIbα-dependent PAR signalling in mouse platelets (FIG. 9D).

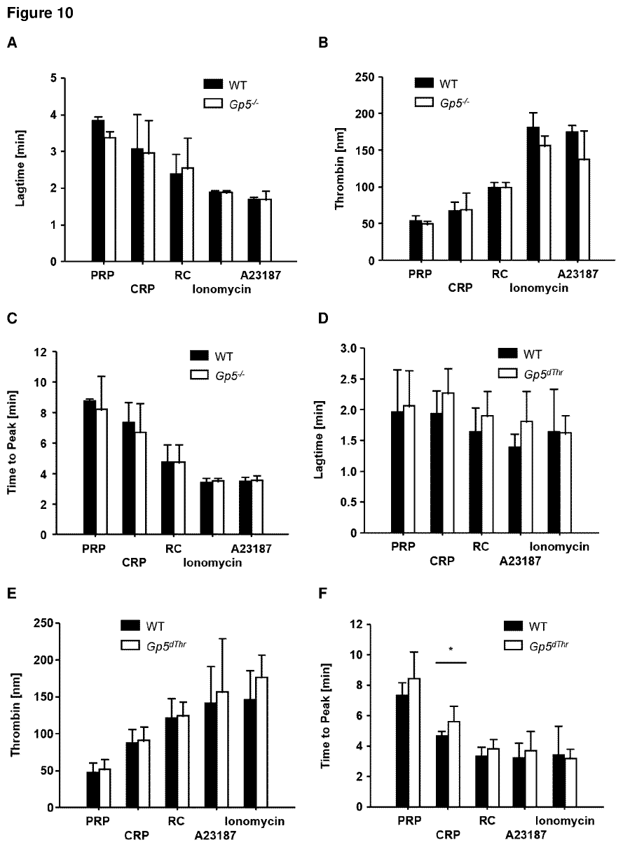

The delineated pathway of enhanced in vitro thrombin signalling in Gp5^{-/-} platelets could not explain the similar prothrombotic phenotype of Gp5^{-/-} and Gp5^{dThr} mice in vivo, suggesting that shed GPV regulated thrombus formation by a mechanism unrelated to the regulation of platelet activation following vascular injury. The present inventors therefore next asked whether platelet procoagulant function might be regulated by GPV. Measurements of TF-initiated thrombin generation in platelet-rich plasma did, however, not uncover differences between Gp5^{-/-}, Gp5^{dThr}, and WT platelets (FIG. 10), in line with previous results with GPV-deficient platelets¹², excluding alterations in platelet membrane procoagulant activity.

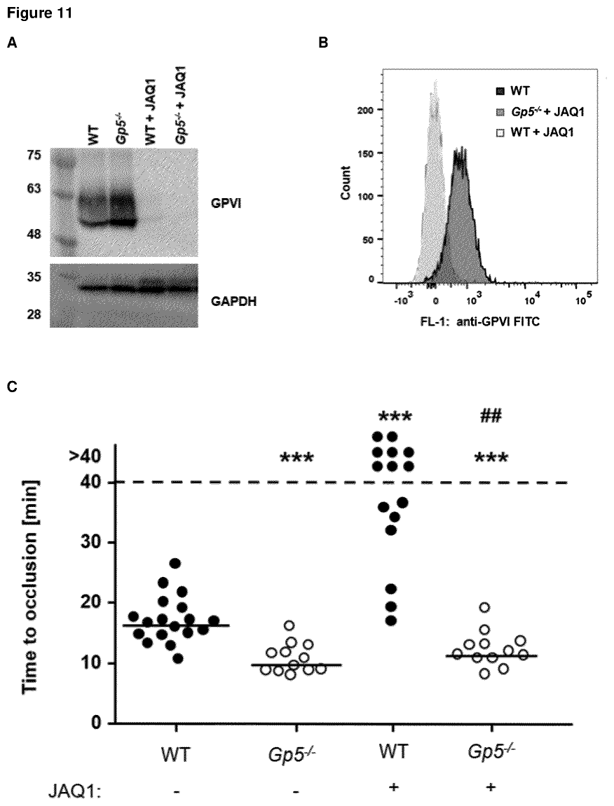

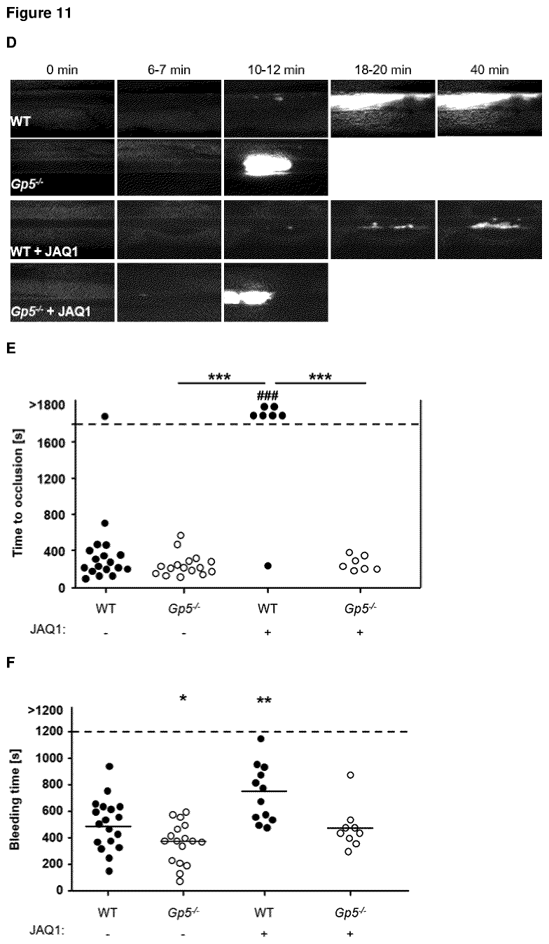

The present inventors next evaluated whether the known collagen interaction of GPV might contribute to the thrombus growth modulation by GPV. Platelet activation is triggered through two major signalling pathways. Specifically, soluble agonists, including thrombin and secondary mediators ADP and thromboxane A2, act through G protein-coupled receptors (GPCRs), whereas immobilised/multimeric ligands signal through immunoreceptor tyrosine-based activation motif (ITAM) coupled receptors, C-lectin like receptor 2 (CLEC-2) and GPVI. Platelet GPVI is the major activating collagen receptor and GPVI deficiency and antagonism protects from arterial thrombosis with more moderate effects on haemostasis³⁰. The present inventors analysed thrombus formation in the absence or presence of platelet GPVI to uncover potential collagen binding functions of GPV. GPVI was immunodepleted from platelets by injection of the anti-GPVI antibody, JAQ1,³¹ 5 days before inducing the FeCl₃ mesenteric arteriole injury (FIG. 11A, B). As reported previously³¹, GPVI depletion markedly attenuated occlusive thrombus formation in WT mice in vivo. Surprisingly, loss of GPVI was without effect in the absence of GPV and the shortened occlusion times of Gp5^{-/-} mice persisted even after GPVI depletion in two distinct vascular beds (FIG. 11 C-E).

In addition, GPV deficiency prevented the prolongation of the bleeding time associated with GPVI depletion in WT animals (FIG. 11F). These data essentially excluded that GPV regulated GPVI-collagen interaction or contributed to collagen-dependent platelet activation under these experimental conditions. Rather, GPV deficiency overruled the haemostatic and thrombotic defects caused by the absence of GPVI and restored thrombus formation in vivo. It has previously been shown that functional defects related to GPVI-ITAM-mediated platelet activation can be attenuated by increased local thrombin generation in different vascular beds³² and mouse GPVI does not interact with mouse fibrinogen³³. Thus, the demonstrated reversal of GPVI inhibition in Gp5^{-/-} mice suggested that soluble GPV (sGPV) regulated thrombin activity during thrombus formation.

sGPV Binds to Thrombin and Localises to Fibrin

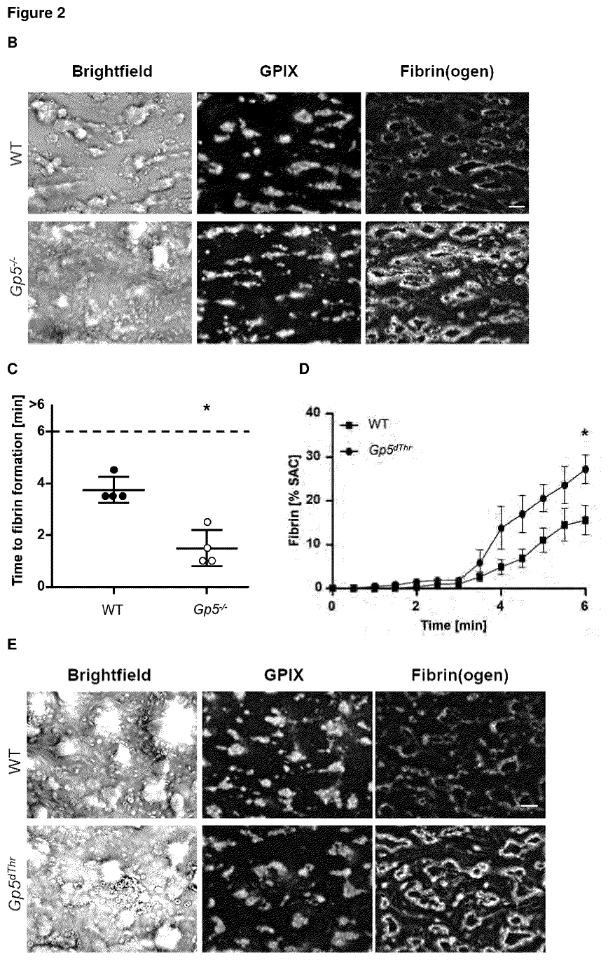

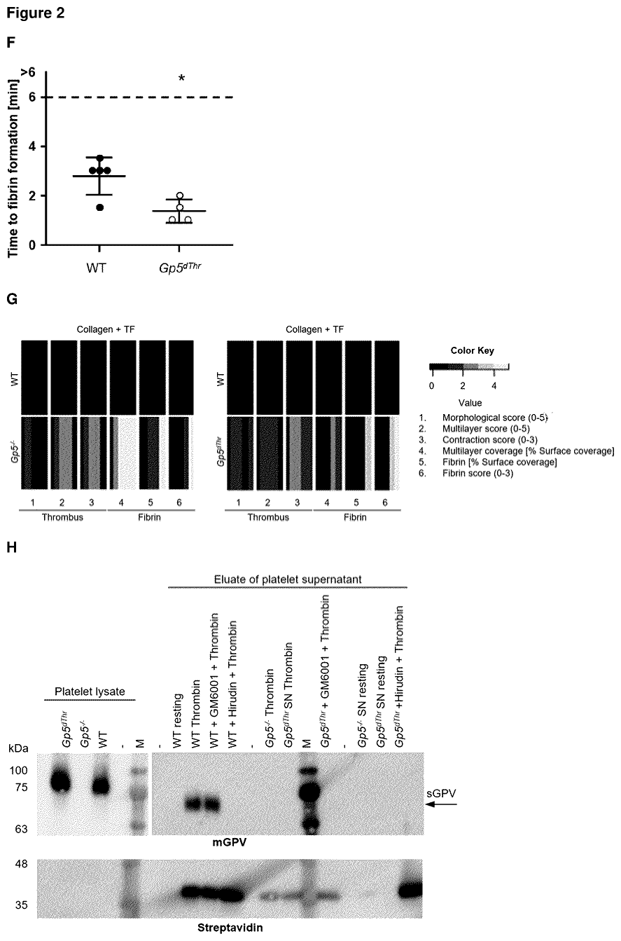

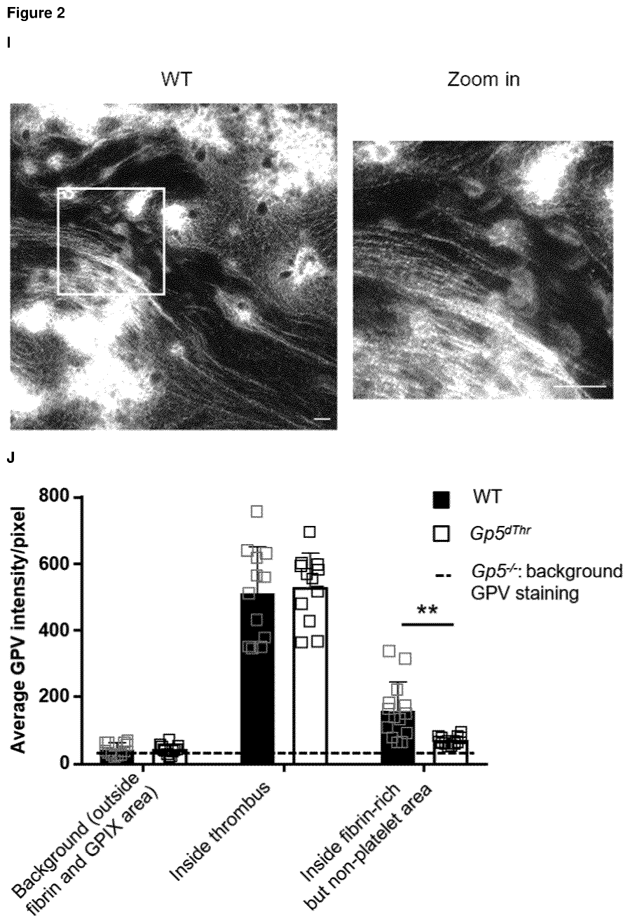

The present inventors therefore evaluated the role of GPV in thrombin-mediated fibrin formation on collagen/TF spots in recalcified whole blood under flow in vitro³⁴. Time to fibrin formation was shortened and the overall amount of fibrin generated was increased in Gp5^{-/-} mice (FIG. 2A-C) and, importantly, also in Gp5^{dThr} mice (FIG. 2D-F) compared to WT controls. Quantitative imaging of formed thrombi and generated fibrin³⁵ showed increased thrombus height, based on multilayer and contraction scores, as well as fibrin formation, based on fibrin surface coverage and fibrin score, in the blood of both mutant mouse lines (FIG. 2G). Of note, this ex vivo experimental setup produced results entirely in line with the in vivo findings that Gp5^{-/-} and Gp5^{dThr} mice concordantly displayed accelerated thrombus formation.

These data indicated that cleavage of GPV is a critical step in an autoregulatory limitation of fibrin generation. Thrombin binds to de novo generated fibrin via the regulatory thrombin exosites I and II and thereby becomes protected from coagulation inhibitors in the blood³⁶. The present inventors hypothesised that sGPV directly or indirectly affected thrombin-fibrin interactions. The present inventors first evaluated the direct interaction of sGPV and thrombin. The present inventors stimulated platelets with biotinylated thrombin and showed that sGPV coprecipitated in the thrombin pull down using streptavidin-coated beads (FIG. 2H), consistent with direct interaction of thrombin with sGPV.

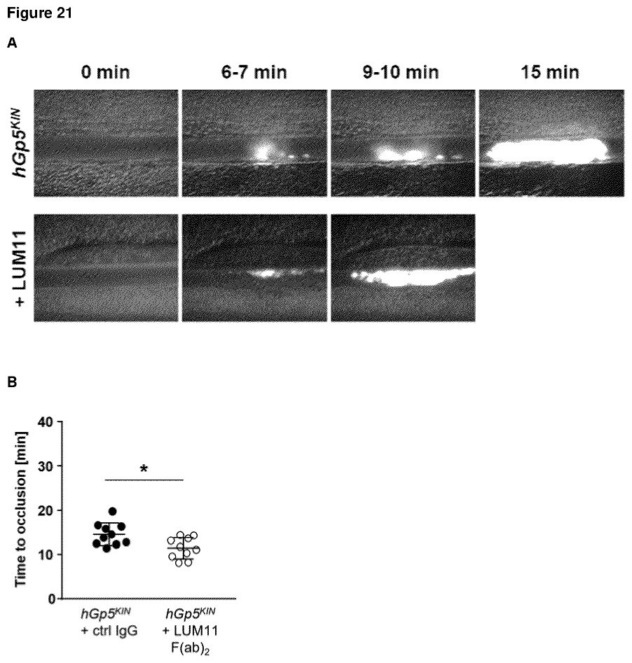

Because GPV release was required to attenuate fibrin formation of recalcified whole blood perfused over collagen/TF spots (FIG. 2A-F), the present inventors next quantified the colocalization of GPV with fibrin in this setting. In confocal microscopy with super-resolution mode, the present inventors excluded in the image analysis platelet-rich areas based on GPIX staining and quantified subsequently the colocalization of GPV with fibrin (FIG. 21, J, 12). Quantification of GPV intensities showed that GPV accumulated with fibrin in platelet-free areas of thrombi (FIG. 2J).

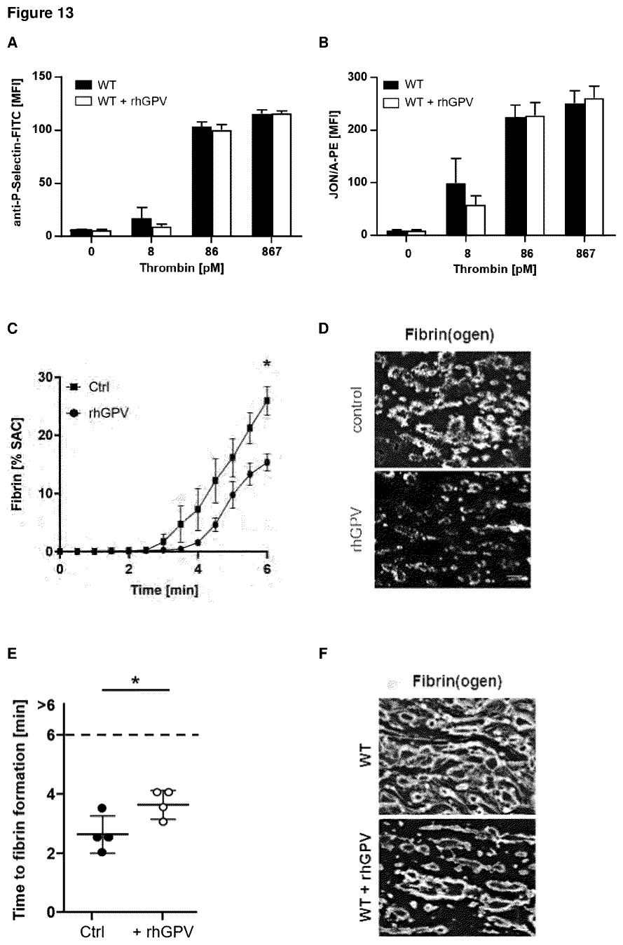

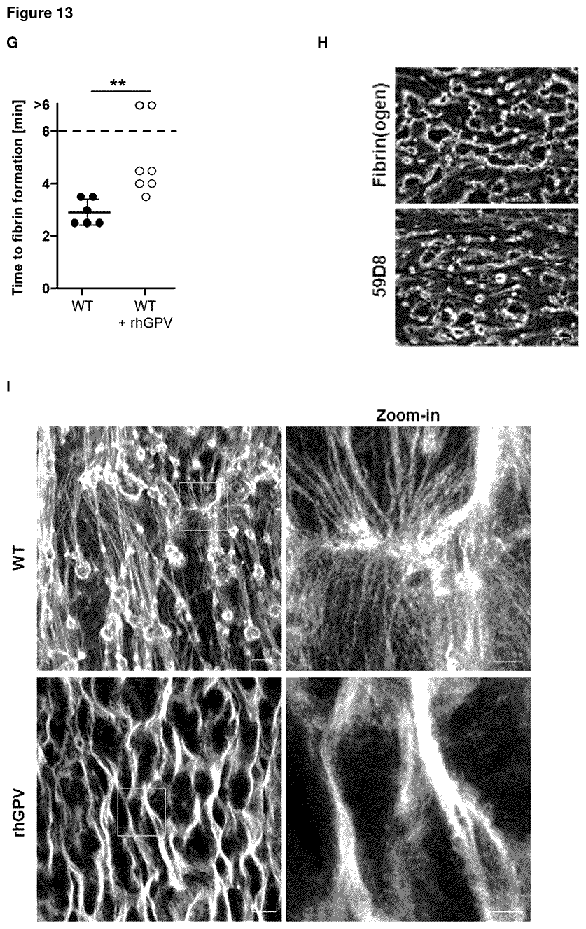

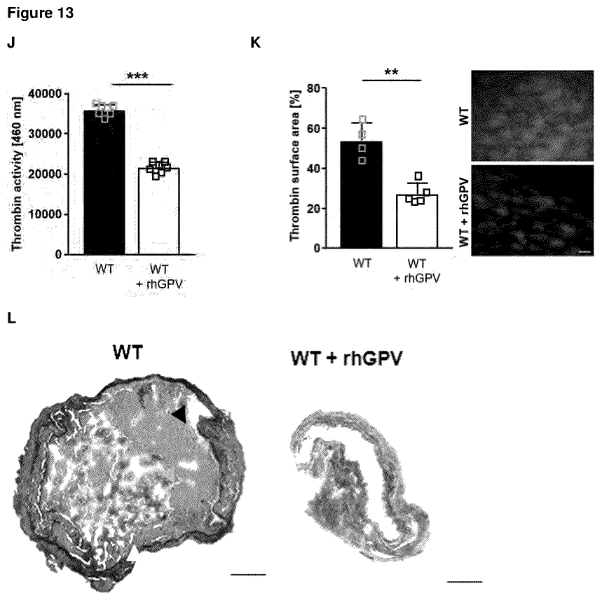

Based on these data, the present inventors reasoned that upon initiation of a haemostatic platelet response, thrombin-mediated cleavage of GPV formed sGPV-thrombin complexes, which limited thrombin diffusion and activity in the forming fibrin clot. To test this concept, the present inventors recombinantly expressed the ectodomain of human GPV in a construct that included the thrombin cleavage site (rhGPV) (FIG. 3A). Aggregation of rhGPV at high concentrations prevented us from performing experiments with full dose response curves. However, thrombin-mediated platelet activation was only marginally inhibited by 290 nM (20 μg/ml) rhGPV at threshold thrombin concentrations (FIG. 13A, B), in line with the conclusion that platelet activation by thrombin is primarily regulated by membrane bound GPV. In sharp contrast, rhGPV at the same concentration impaired fibrin formation in a static polymerization assay (FIG. 3B) triggered specifically by thrombin, whereas fibrin polymerization induced by another protease, batroxobin, was unaltered in the presence of rhGPV (FIG. 3B). Importantly, sGPV localized to fibrin polymers independent of the clot inducing enzyme, indicating direct interactions of GPV with fibrin independent of thrombin-GPV complex formation.

rhGPV Reduces Fibrin Formation and Protects from Thrombosis

In addition, rhGPV impaired fibrin formation in human (FIG. 13C-E) and mouse blood (FIG. 13F-H) in the collagen/TF-induced thrombus formation assay under flow, supporting a role for sGPV in limiting thrombin activity towards fibrin. Analysis of the formed fibrin fibrils by confocal microscopy revealed a fine, dense, and branched network consisting of thin, clearly distinguishable fibres in control samples, whereas fibres were generally thicker, but less frequently and structurally less defined in the presence of rhGPV (FIG. 13I), confirming that rhGPV impedes fibrin formation.

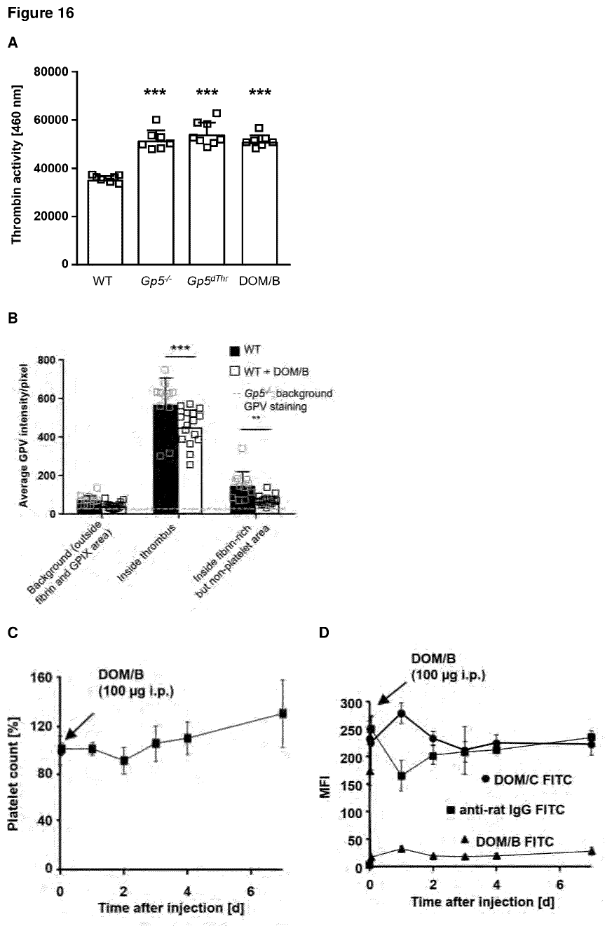

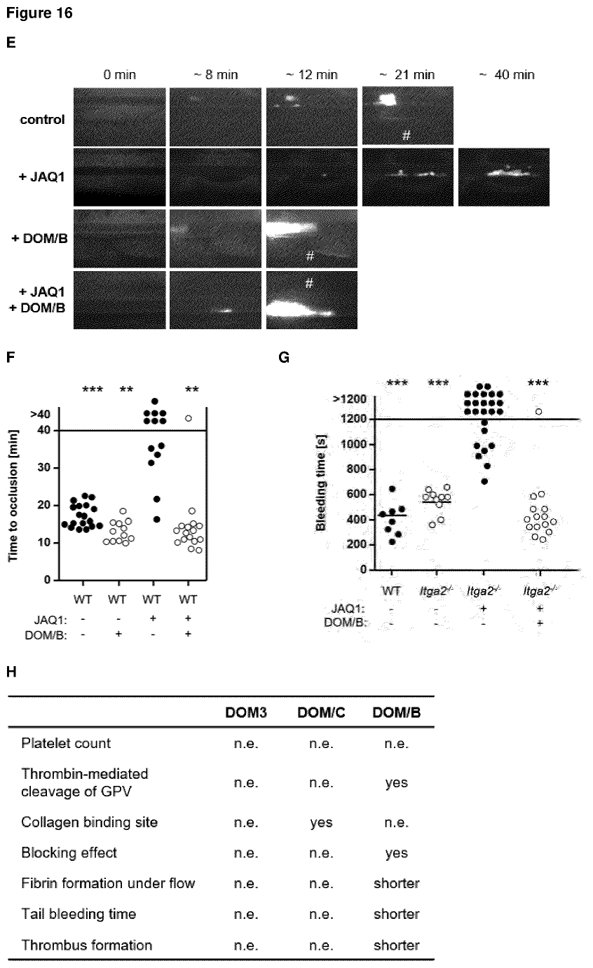

The present inventors next measured thrombin activity in the outflow of the flow chamber and found that less thrombin activity was recovered in rhGPV-treated samples compared to controls (FIG. 13J). Conversely, the present inventors found more thrombin in the outflow of the chambers perfused with Gp5^{-/-} and Gp5^{dThr} versus WT blood (FIG. 16A), further supporting the conclusion that sGPV controlled thrombin activity specifically in fibrin clots. The present inventors next imaged thrombin activity in flow chambers cleared of blood by perfusion with Tyrode's buffer and thrombin substrate Z-GGR-AMC. The present inventors found reduced thrombin activity in clots formed in the presence of rhGPV (FIG. 13K). Taken together, these data support a role for GPV in retaining thrombin in fibrin clots and limiting thrombin's activity in fibrin formation.

The present inventors next tested whether rhGPV could modulate thrombus formation in vivo. Indeed, a single intravenous dose of 20 μg rhGPV prior to thrombosis induction reduced arterial thrombus formation in two different experimental models. In a model of mechanical injury to the abdominal aorta where blood flow and occlusive thrombus formation was monitored by an ultrasonic flow probe (FIG. 3C, 13L), 14/15 mice did not form stable thrombi after rhGPV administration within the observation period of 30 minutes, whereas 18/18 arteries occluded in the control group. In FeCl₃-induced mesenteric arteriole injury, time to occlusion was markedly prolonged in mice treated with rhGPV (FIG. 3D, E).

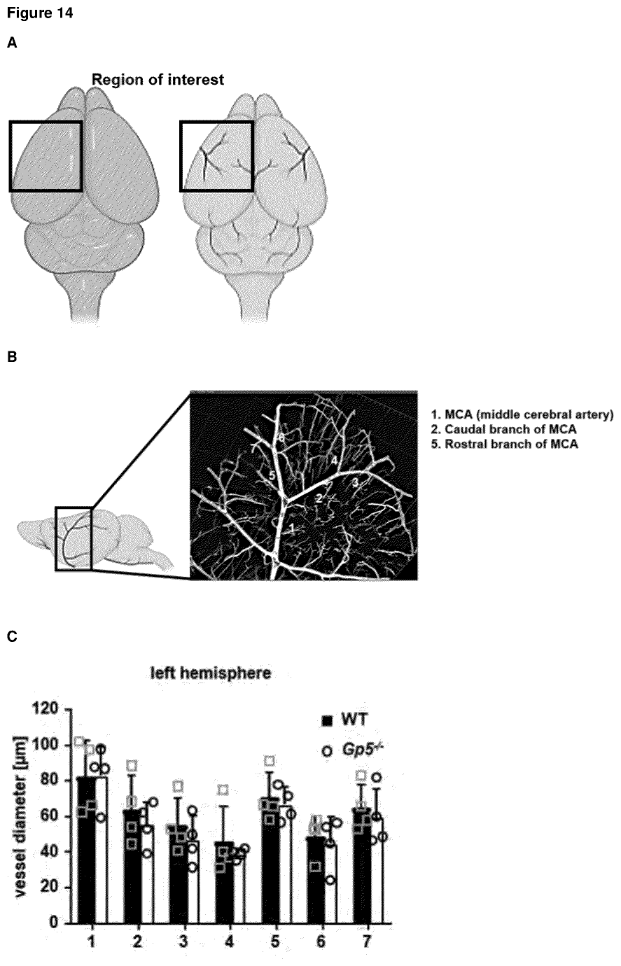

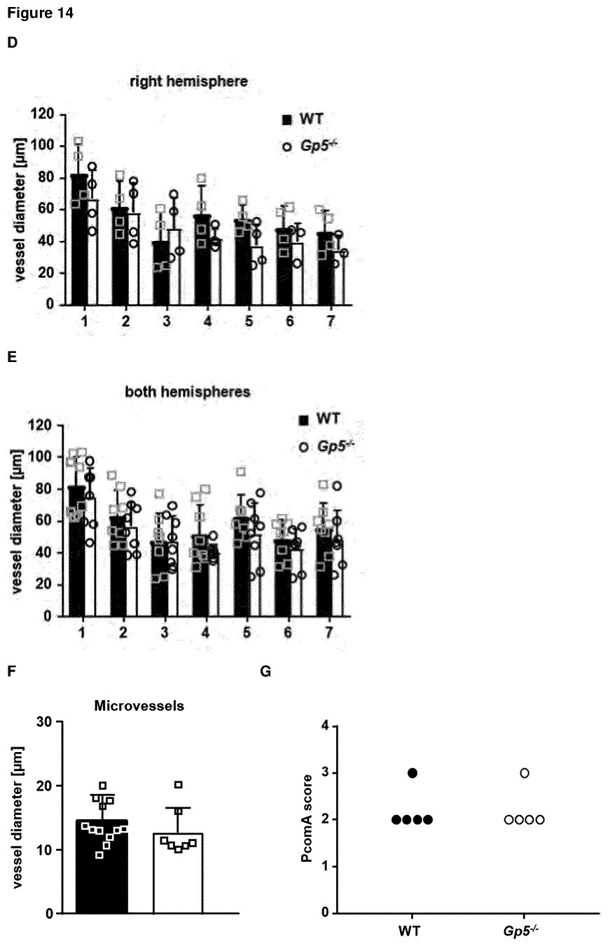

In addition, rhGPV treatment provided protection from thrombo-inflammatory neurological damage and improved neurological outcome in the transient middle cerebral artery occlusion (tMCAO) model of ischaemic stroke (FIG. 3F-H) in which the concerted action of platelets, the coagulation system and immune cells is known to drive post-ischaemic cerebral infarct growth³⁷. Of note, infarct volumes of Gp5^{-/-} and Gp5^{dThr} mice after tMCAO were comparable to WT mice (FIG. 3F-G), suggesting that thrombin activity in WT mice is already above threshold values needed to fully promote infarct progression under these experimental conditions. Importantly, no large intracranial haemorrhages were observed in Gp5^{-/-} or Gp5^{dThr} and rhGPV-treated WT mice (FIG. 3G). Of note, MCA vessel diameters were similar in Gp5^{-/-} and WT mice (FIG. 14). In addition, haemostatic function evaluated by tail bleeding time assay was also comparable between rhGPV-injected mice and vehicle-treated controls (FIG. 3l), indicating that this pathway might be targeted safely. Together, these data showed that sGPV specifically limited fibrin formation and pathological intravascular thrombus growth without impairing initial platelet activation required for haemostasis.

### Blockade of Thrombin-Mediated GPV Cleavage Offsets Severe Defects in Haemostatic Platelet Function

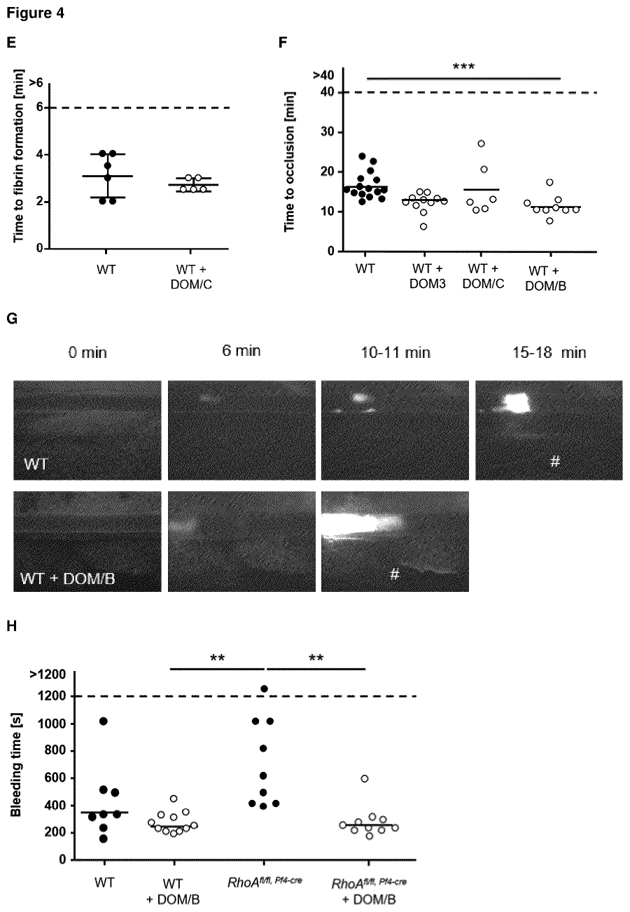

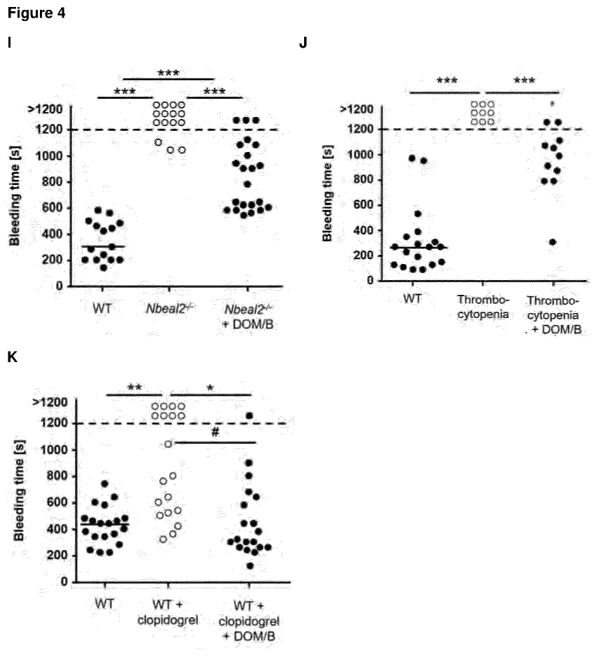

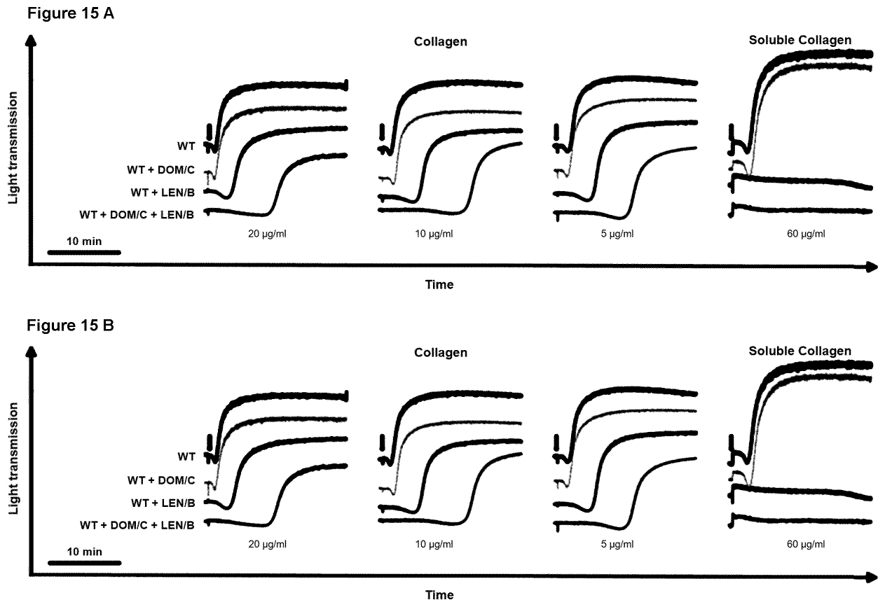

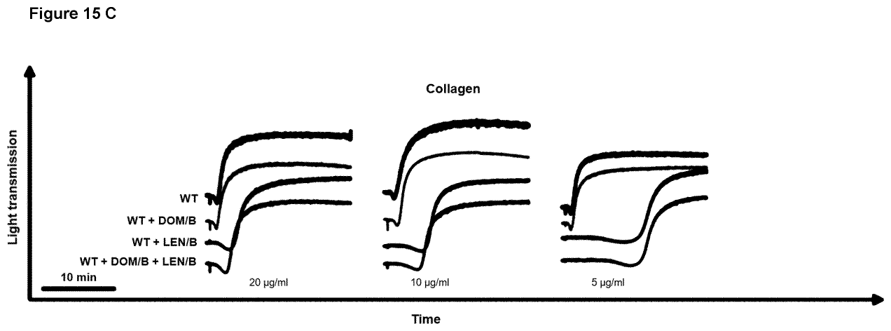

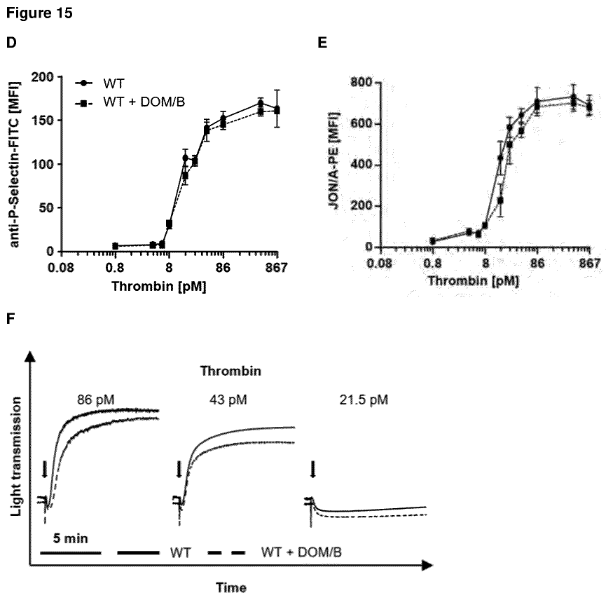

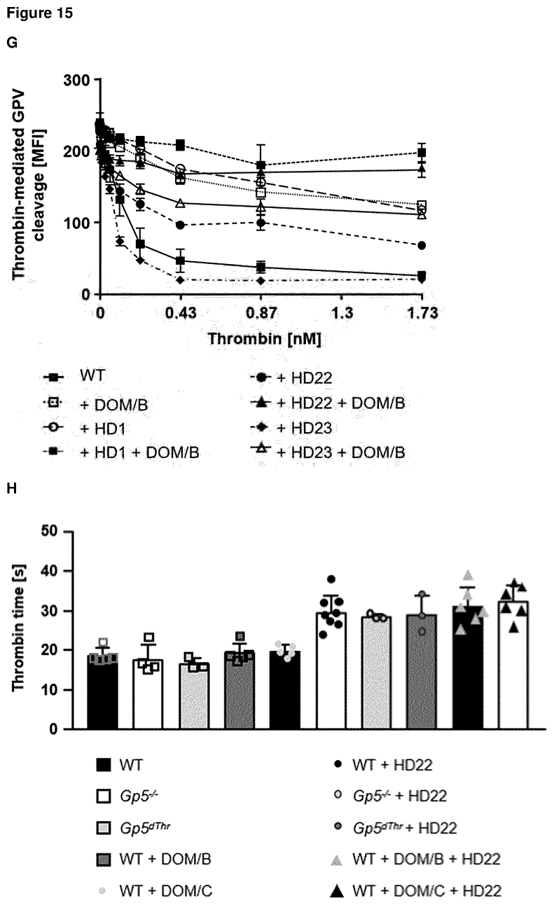

To further study thrombin interaction with GPV, the present inventors generated a panel of anti-GPV monoclonal antibodies (termed DOM mAbs; FIG. 15) and first evaluated their ability to inhibit thrombin mediated GPV cleavage (FIG. 4A). Cleavage of substrates by thrombin involves binding and allosteric regulation by thrombin exosites I and II that flank the active site³⁸. Blockage of exosite I with the thrombin binding aptamer HD1³⁹ was more efficient than blocking exosite II with HD22⁴⁰, whereas a non-blocking aptamer HD23 was without effect on thrombin-mediated release of GPV from platelets (FIG. 15G). Thus, thrombin interaction with fibrin and GPV occurred through overlapping sites³⁶. With this screening assay, the present inventors identified mAb DOM/B that markedly reduced thrombin-mediated GPV cleavage and synergised with thrombin exosite-directed aptamers (FIG. 4A, 15G), whereas mAb DOM3 was non-inhibitory (FIG. 4A, F, 15B). In platelet-rich plasma, the present inventors tested inhibitory activities of DOM/B in a thrombin-induced clotting assay, in which fibrin is formed independent of platelet activation. Whereas thrombin exosite II blockade with HD22 prolonged clotting times, clotting was unaffected by DOM/B-treatment as well as was indistinguishable between WT, Gp5^{-/-} and Gp5^{dThr} samples (FIG. 15H). Thus, thrombin regulation by GPV specifically occurs under conditions of platelet activation under flow.

In addition, DOM/B had no effect on thrombin-induced platelet activation (FIG. 15D-F), indicating that this mAb did not sterically hinder the interaction of GPV with the GPIb-IX complex involved in GPIbα-thrombin-PAR platelet signalling. Remarkably, however, DOM/B significantly shortened time to fibrin formation and increased the amount of generated fibrin under flow conditions (FIG. 4B, C) as well as the thrombin activity in the outflow of the flow chamber (FIG. 16A), thereby reproducing the phenotypes seen with Gp5^{-/-} and Gp5^{dThr} mice. In line with reduced proteolytic release of GPV in the presence of DOM/B, the present inventors found less GPV colocalizing with fibrin compared to controls (FIG. 16B). The panel of anti-GPV mAbs was also evaluated for interference with collagen-dependent platelet activation. Whereas DOM/B and DOM3 were non-inhibitory (FIG. 15B, C), inhibition of platelet activation in this assay by DOM/C indicated that this mAb was directed against the collagen binding site of GPV (FIG. 15A). DOM/C did not interfere with thrombin mediated GPV cleavage (FIG. 4A), and did not enhance fibrin formation under flow, consistent with the crucial and specific role of sGPV release in this context (FIG. 4D, E). This data suggested that the collagen binding activity of (s)GPV is functionally not required for its ability to modulate fibrin formation under flow in vitro and in vivo.

The present inventors next evaluated the effect of blocking GPV-thrombin interaction with DOM/B on fibrin and thrombus formation in vivo. Of note, injection of DOM/B did not cause platelet depletion and the mAb remained detectable on the surface of circulating platelets for up to 6 days (FIG. 16C, D). In line with the observed increased fibrin formation under flow in vitro, DOM/B treatment caused accelerated thrombus formation in FeCl₃-injured mesenteric arterioles in vivo, whereas neither blockade of the collagen binding site on GPV with DOM/C nor the non-inhibitory DOM3 affected thrombus formation (FIG. 4F, G). These data showed that the release of sGPV acts as a safety valve to limit thrombus growth after initial platelet activation required for haemostasis. The present inventors have previously shown that the absence of the two major collagen receptors GPVI and integrin α2β1 causes severe bleeding in mice⁴¹. In line with the reversal of bleeding defects caused by GPVI-deficiency in Gp5^{-/-} mice, DOM/B-treatment restored haemostasis and thrombus formation in the complete absence of the two major platelet collagen receptors GPVI and α2β1 (FIG. 16E-G, summarized in 16H).

These results suggested that interference with GPV cleavage can be utilised to enhance fibrin formation in the context of defective haemostasis caused by diverse mechanisms. The present inventors therefore investigated whether GPV cleavage blockade with DOM/B not only restored haemostasis in the case of defective (hem)ITAM signalling, but also other genetic and pharmacological impairments of platelet function. Lack of the small GTPase RhoA causes macrothrombocytopenia and defective platelet activation, resulting in a bleeding defect⁴². Similarly, Nbeal2-deficiency leads to macrothrombocytopenia and lack of α-granules resulting in severely impaired haemostasis⁴³. Interestingly, DOM/B-treatment improved and restored haemostasis in mice with platelet RhoA deficiency or lacking Nbeal2 (FIG. 4H, I).

Thrombocytopenia is a major clinical challenge occurring frequently in the context of a variety of pathologies or medical treatments that is associated with increased bleeding and often with the need of immediate therapeutic intervention^{44,45}. To test a possible benefit of a GPV cleavage blockade in this setting, the present inventors induced severe thrombocytopenia by reducing platelet counts to 5-10% of normal by injecting a platelet-depleting antibody^{46,47}. While a resulting severe bleeding defect was observed in all 9 platelet-depleted control mice, this was significantly attenuated by DOM/B-treatment, and remarkably 9/11 DOM/B-treated thrombocytopenic mice managed to stop bleeding within the observation period (FIG. 4J). The current clinical standard of care to reduce the risk of heart attack and ischaemic stroke is the pharmacological inhibition of platelet function by P2Y₁₂ ADP receptor blockers alone or in combination with acetyl salicylic acid (ASA). As seen in humans, mice treated with the P2Y₁₂-blocker clopidogrel exhibit increased bleeding that was reversed by treatment with DOM/B blocking thrombin-dependent GPV release from platelets (FIG. 4K). Thus, specific targeting of GPV with DOM/B prevented the prolongation of the bleeding time caused by thrombocytopenia, genetic defects, and anti-platelet therapy, indicating clinical potential of anti-GPV treatment to restore haemostasis by improving thrombin-dependent fibrin formation.

### Specific Blockade of Thrombin-Mediated GPV Cleavage in Human Blood Increases Fibrin Formation

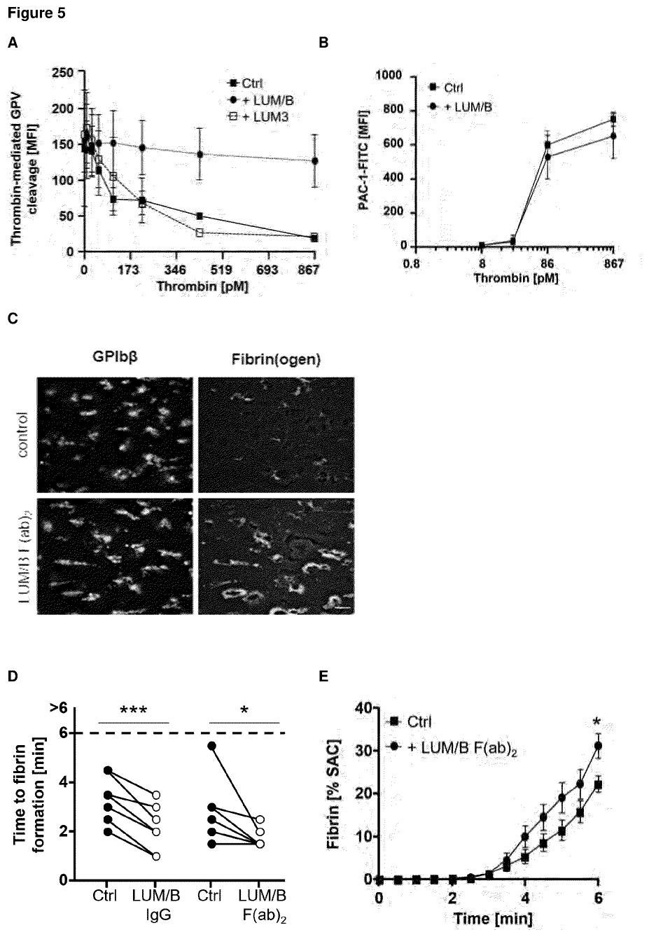

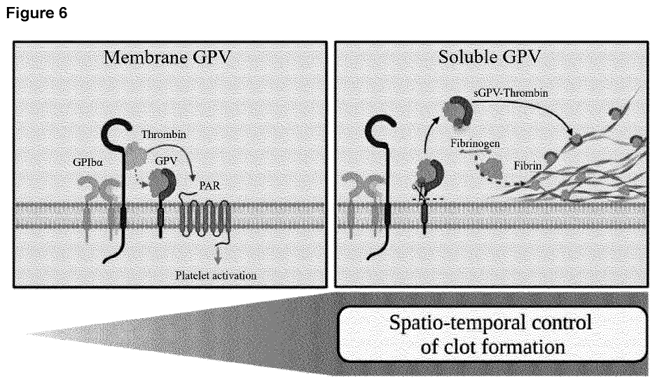

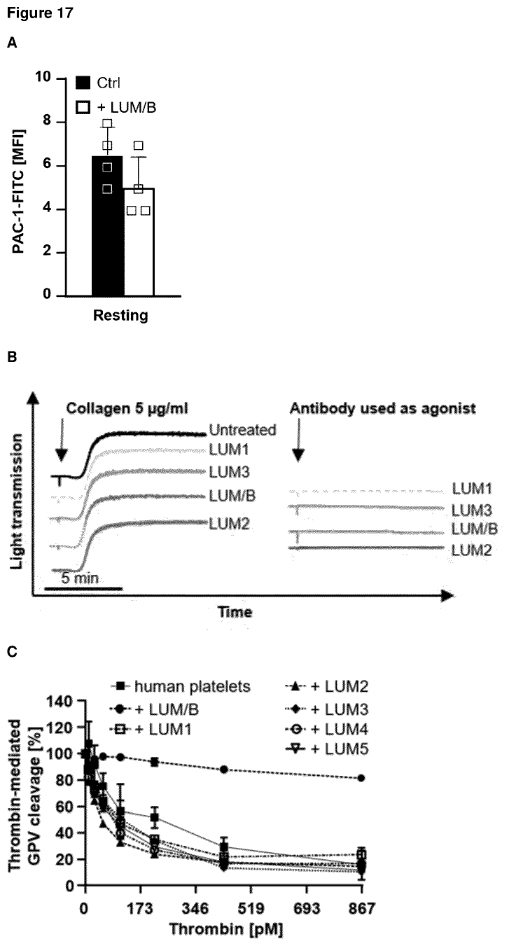

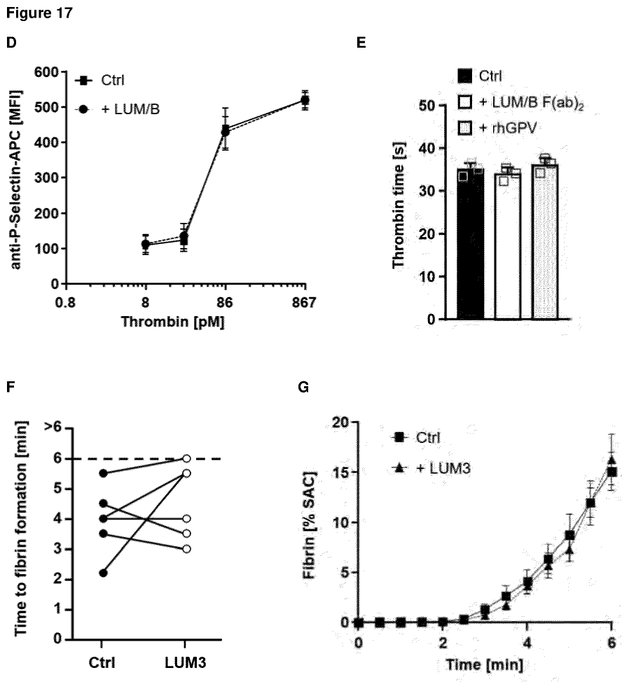

The present inventors therefore evaluated the relevance of this concept for human platelet function and screened a panel of newly generated anti-hGPV mAbs (termed LUM mAbs) for their ability to interfere with thrombin-mediated cleavage of GPV. Recapitulating the inhibitory properties of DOM/B in the mouse system, LUM/B prevented thrombin-mediated cleavage of GPV on human platelets, whereas other anti-hGPV mAbs (LUM1-5) were non-inhibitory (FIGS. 5A and 17C). LUM/B per se neither activated human platelets (FIG. 17A, B) nor influenced thrombin clotting (FIG. 17E) nor thrombin-induced platelet activation as shown by unaltered integrin αIIbβ3 activation or P-selectin exposure in the presence of the antibody (FIG. 5B, 17D). In the established flow assay on immobilised collagen/TF, GPV blockage on human platelets with LUM/B significantly accelerated fibrin formation in recalcified whole blood (FIG. 5C-E), whereas the non-inhibitory LUM3 was without effect (FIG. 17F, G). Thus, spatio-temporal control of fibrin formation on thrombogenic surfaces by GPV is a species-conserved mechanism to restrict thrombosis while preserving haemostasis.

Exact p-Values

FIG. 1a: p<0.0001; FIG. 1c: p=0.0010; FIG. 1e: 4.3 μM thrombin: p=0.0025, 6.45 μM thrombin: p=0.0286, 8.6 μM thrombin: p=0.0020, 17.2 μM thrombin: p=0.0179.; FIG. 1h: WT vs. Gp5^{-/-} and Gp5^{-/-} vs. Gp5^{dThr}: p<0.0001.

FIG. 2a: 4.5 min: p=0.0072, 5.5 min: p=0.0296, 6 min: p=0.0006; FIG. 2c: p=0.0286; FIG. 2d: 6 min: p=0.0287; FIG. 2f: p=0.0317; FIG. 2j: p=0.0086.

FIG. 3c: p<0.0001; FIG. 3e: open vs. occluded vessels: WT vs. rhGPV: p<0.0108, Comparison of occluded vessels: p=0.002; FIG. 3f: p=0.0298; FIG. 3h: WT vs. WT+rhGPV: p=0.0135, WT+rhGPV vs. Gp5^{-/-}: p=0.0116, WT+rhGPV vs. Gp5^{dThr}: p=0.0440.

FIG. 4c: p=0.0010; FIG. 4f: DOM/B vs. WT: p=0.0008; FIG. 4h: RhoA^{fl/fl Pf4-cre}+DOM/B vs. RhoA^{fl/fl Pf4-cre}: p=0064; DOM/B vs. RhoA^{fl/fl Pf4-cre}: p=0.002; FIG. 4i: Open vs. occluded vessels: WT vs. Nbeal2^{-/-}: p<0.0001, Nbeal2^{-/-}+DOM/B vs. Nbeal2^{-/-}: p<0.0001, comparison of occluded vessels: Nbeal2^{-/-}+DOM/B vs. WT: p<0.0001; WT vs. Nbeal2^{-/-}: p=0.0006; FIG. 4j: Open vs. occluded vessels: WT vs. thrombocytopenic mice: p<0.0001, thrombocytopenic mice+DOM/B vs. thrombocytopenic mice: p=0.0003, Comparison of occluded vessels: thrombocytopenic mice+DOM/B vs. WT: p=0.0251; FIG. 4k: Open vs. occluded vessels: WT vs. WT+clopidogrel: p=0.0031, WT+DOM/B+clopidogrel vs. WT+clopidogrel: p=0.0188, Comparison of occluded vessels: WT+DOM/B+clopidogrel vs. WT+clopidogrel: p=0.0383.

FIG. 5d: Ctrl vs. 1B1 IgG: p=0.006; Ctrl vs. 1B1 F(ab)2: p=0.0313; FIG. 5e: 6 min: p=0.02.

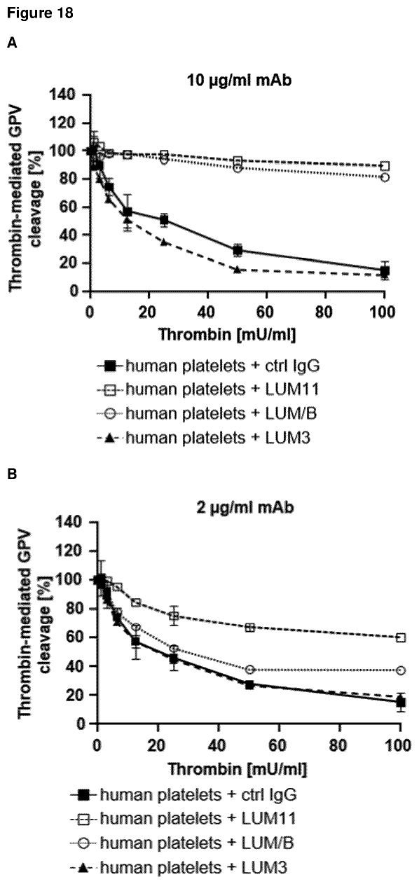

### Example 2.1—Methods for Experiments for FIGS. 18 to 21

### Animals

Mice were maintained under specific pathogen-free conditions (constant temperature of 20-24° C. and 45-65% humidity with a 12-h light-dark cycle, ad libitum water and food access) and experiments were performed in accordance with German law and the governmental bodies, and with approval from the District of Lower Franconia.

hGp5^{KIN} mice were generated by replacing the extracellular domain of murine GPV by the human All mice were kept on a C57BI/6J background, and all animal experiments and the analysis of the corresponding data were performed blinded.

### Human Blood Samples

For this study, blood samples were obtained from healthy volunteers, free from anticoagulant or anti-platelet therapy for at least 4 weeks, following written informed consent in accordance with the Declaration of Helsinki and after approval by the Institutional Review Boards (IRB) of University of Wurzburg.

### Antibodies

Control rat IgG (#14131) was from Sigma. The platelet-depletion antibody R300 (rat anti-GPIbα IgG antibody) was from Emfret Analytics (Eibelstadt, Germany).

Table 4. In house generated antibodies

| | | | |
|---|---|---|---|
| Mouse GPIX | pp6 | In-house generated | 54 |
| Mouse/human GPlbß | p0p1 | In-house generated | 57 |
| Human GPV | LUM/B | In-house generated | (see above) |
| Human GPV | LUM1 | In-house generated | |
| Human GPV | LUM11 | In-house generated | |

LUM11 was generated by hybridoma technology following immunisation of Wistar rats with recombinant hGPV protein and GPV immunoprecipitated from human platelet lysates.

### Antibody Treatment

For the antibody treatment experiments the mice were separated into antibody and control treatment group in a randomized manner using https://www.random.org/lists/. 100 μg LUM11 (or control IgG) were injected intravenously directly before the experiment.

### Platelet Depletion

Thrombocytopenia was induced by intravenous injection of rat anti-GPIbα IgG antibody R300 (Emfret Analytics, Eibelstadt, Germany, 0.14-0.18 μg/g body weight). This low dose of platelet depletion antibody reduced the platelet count to 5-10% of the initial platelet count⁴⁶. Peripheral platelet count was determined by flow cytometry 16 h after platelet depletion (prior to tail bleeding time experiment).

Treatment with Clopidogrel

Mice were fed orally with 3 mg/kg clopidogrel 48 h and 24 h before the experiment.

### Flow Cytometry

To determine glycoprotein surface expression levels, whole blood was diluted 1:20 with Ca²⁺-free Tyrode's buffer or PBS and stained with saturating amounts of fluorophore-conjugated antibodies for 15 min at RT in the dark. Washed platelets adjusted to 50000/μl in Tyrode's buffer with Ca²⁺ were stimulated with the indicated agonists and incubated with saturating amounts of fluorophore-conjugated antibodies to determine platelet activation or thrombin-mediated cleavage of GPV. Human Thrombin (Sigma #10602400001) was used to stimulate platelets. All samples were analysed directly after addition of 500 μl PBS on a FACSCalibur (BD Biosciences, Heidelberg, Germany). Exemplified gating strategy based on FSC/SSC characteristics are shown in FIG. 7.

### Coagulation Flow Chamber

Glass coverslips were coated with collagen type I (10 μl, 50 μg/ml) and tissue factor (TF; 10 μl, 100 pM or 10 pM for experiments with human blood or mouse blood, respectively) and blocked with 1% BSA/PBS. Citrated whole blood was recalcified by co-infusion with 6.3 mM CaCl₂ (f.c.) and 3.2 mM MgCl₂ (f.c.) and perfused over the collagen/TF spots for up to 6 min at a shear rate of 1000 s^{-1 60}. Before each experiment, blood samples were pre-labeled with Alexa Fluor™ (AF) 488-conjugated fibrinogen, an anti-GPIX derivative (mouse) or anti-GPIbβ derivative (human) to stain platelets. For human samples, control and antibody-treated samples of the same donor were always run in parallel.

Before each experiment, blood samples were preincubated with AF488-conjugated fibrinogen, AF647-conjugated anti-GPIX derivative (mouse) or AF647-conjugated anti-GPIbβ derivative (human) platelets. For time series experiments, fluorescence microscopic images were captured at 30 s intervals, to evaluate kinetics for up to 6 min (Leica DMI 6000 B, 63× objective). Recorded images were further processed with the background subtraction method ICC (Instant Computational Clearing) to remove out-of-focus blur on a Leica Thunder microscope. Next, the exported images were analysed for surface area coverage of fibrin formation with self-written Python scripts⁶¹. In detail, in the first step an entropy filter with a disk size of 5 pixel was applied, followed by a median filter (disk size 10 pixel) and Otsu-thresholding. To compensate for a nearly empty field of view during the first images in the time series, the present inventors introduced a “scaling factor” sf with which the present inventors multiplied the found Otsu-threshold value, and thus increased the threshold slightly for the first images (usually the first 60-150 s, with sf=1.03-1.05). The thresholded area (as fraction of the whole field of view) represents the area covered by fibrin.

### Tail Bleeding Time

Mice were anaesthetised by intraperitoneal injection of triple anaesthesia (Dormitor 0.5 μg/g, Midazolam 5 μg/g, and Fentanyl 0.05 μg/g body weight) and a 1-mm segment of the tail tip was removed using a scalpel. Tail bleeding was monitored by gently absorbing blood on filter paper at 20 s intervals without directly contacting the wound site. When no blood was observed on the paper, bleeding was determined to have ceased. The experiment was manually stopped after 20 min by cauterization.

FeCl₃-Induced injury of Mesenteric Arterioles

3 to 4-weeks old mice were anaesthetised, and the mesentery was exteriorised. Arterioles (35-60 μm diameter) were visualised with a Zeiss Axiovert 200 inverted microscope (10×/0.25 air objective) equipped with a 100-W HBO mercury lamp and a CooISNAP-EZ camera (Visitron, Munich, Germany). Endothelial injury was induced by topical application of a 3 mm² filter paper saturated with ferric chloride (FeCl₃; 20%). Adhesion and aggregation of fluorescently labeled platelets (Dylight 488-conjugated anti-GPIX derivative) was monitored for 40 min or until complete occlusion occurred (blood flow stopped for >1 min).

### Data Analysis

The presented results are mean±SD from three independent experiments per group and lines represent mean values, if not stated otherwise. Normal distribution was tested using the Shapiro-Wilk normality test. If passed, p-values were calculated using the two tailed unpaired t-test (2 groups), if the values were not normally distributed, differences between two groups were analysed using the Mann-Whitney two-tailed test. For more than two groups, one-way ANOVA (Kruskal-Wallis test) followed by Dunn's test for multiple comparisons was performed using GraphPad Prism software (V7.05.). Two tailed paired t-test (2 groups, normally distributed), Wilcoxon matched-pairs signed rank test (2 groups, not normally distributed) and Friedman test followed by Dunn's test for multiple comparisons (more than 2 groups, not normally distributed) were used for paired comparisons. For statistical analysis of non-occluded vs. occluded vessels, Fisher's exact t-test was used. P-values <0.05 were considered statistically significant.

### Example 2.2—Results for Experiments of Example 2.1

In view of e.g. the above experiments, it could be shown that LUM11 is a monoclonal rat IgG that potently inhibits thrombin-mediated GPV cleavage and supports haemostasis in a GPV-humanised mouse model (hGp5KIN).

In particular, prevention of thrombin-mediated cleavage of GPV by anti-hGPV mAb LUM11 accelerates and increases fibrin formation.

In the present inventor's first set of experiments (see examples 1.1 and 1.2 above), the present inventors delineate an unexpected spatio-temporal control mechanism of thrombin activity that is platelet orchestrated and locally limits excessive fibrin formation after initial haemostatic platelet deposition. During platelet activation, the abundant platelet glycoprotein (GP) V is cleaved from the platelet surface by thrombin. Thrombin-mediated shedding of GPV specifically limits thrombin-dependent formation of fibrin after initial platelet deposition. The anti-mouse GPV antibody DOM/B interfered with thrombin-mediated cleavage of GPV, increased fibrin formation and rescued pharmacologic defects in haemostatic platelet function, indicating that the spatio-temporal control of thrombin-dependent fibrin generation also represents a potential therapeutic target to improve haemostasis.

Based on these results, the present inventors therefore evaluated the relevance of this concept for human platelet function and screened a panel of newly generated anti-hGPV mAbs (termed LUM mAbs) for their ability to interfere with thrombin-mediated cleavage of GPV. Recapitulating the inhibitory properties of DOM/B in the mouse system, two anti-human GPV antibodies prevented thrombin-mediated cleavage of GPV on human platelets (LUM/B, LUM11), whereas other anti-hGPV mAbs (LUM1-5) were non-inhibitory (FIG. 18 A).

At high antibody concentrations (10 μg/ml mAb in vitro), LUM/B and LUM11 prevented thrombin-mediated cleavage of GPV to a similar extent (FIG. 18A). However, LUM11 was significantly more potent than LUM/B and very effectively inhibited thrombin-mediated cleavage of hGPV at lower antibody concentrations down to 2 μg/ml (FIG. 18 B). LUM11 is a monoclonal rat anti-human GPV antibody. It was generated by fusion of immortalized AG14 myeloma cells and spleen cells of rats, which had been repeatedly immunized with recombinant human GPV protein.

In a coagulation flow assay on immobilised collagen/tissue factor (TF) to study thrombin-mediated fibrin formation, blockage of GPV cleavage on human platelets with LUM11 significantly accelerated and increased fibrin formation in recalcified whole blood (FIG. 18C-E). Again, LUM11 was more potent compared to LUM/B since it increased fibrin formation more effectively (FIG. 18D, LUM11: 186% fibrin surface coverage compared to control IgG vs. LUM/B: 134% fibrin surface coverage compared to control IgG).

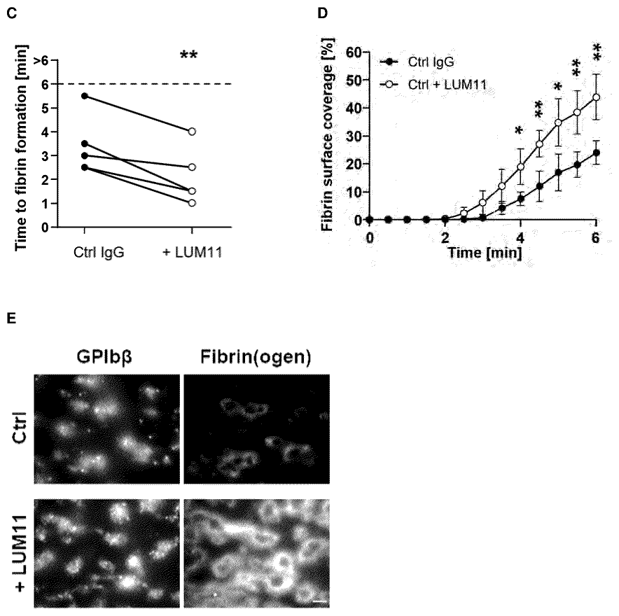

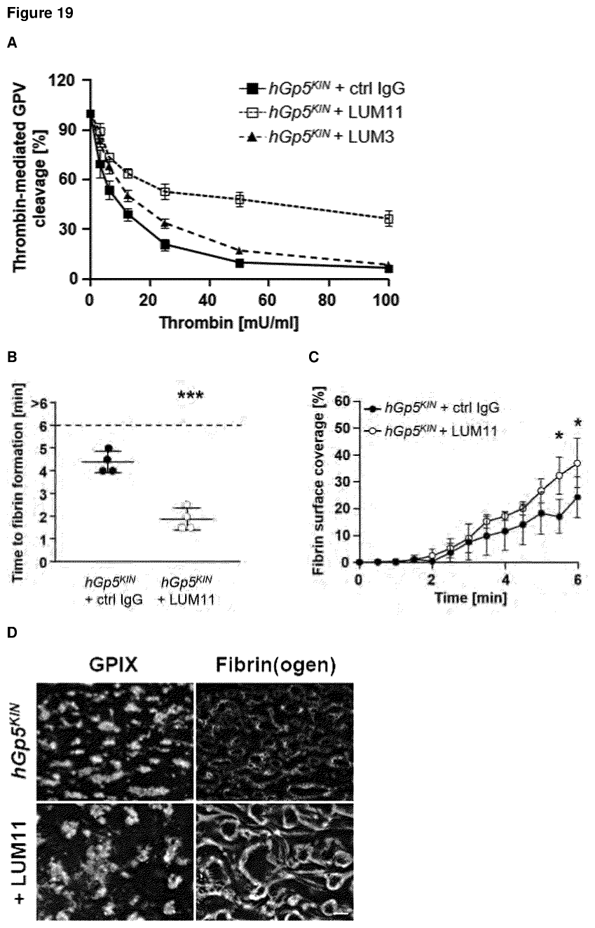

LUM11 was studied in more detail in a humanized GPV mouse model (hGp5^{KIN}). Here, the extracellular domain of murine GPV was replaced by the human sequence (transmembrane and intracellular domain remained mouse GPV). Similar to human blood, LUM11 interfered with thrombin-mediated cleavage of GPV in hGp5^{KIN} platelets (FIG. 19A) and consequently accelerated, and increased thrombin-mediated fibrin formation in recalcified hGp5^{KIN} whole blood on collagen/TF microspots under flow (FIG. 19B-D), recapitulating its effects on human platelets.

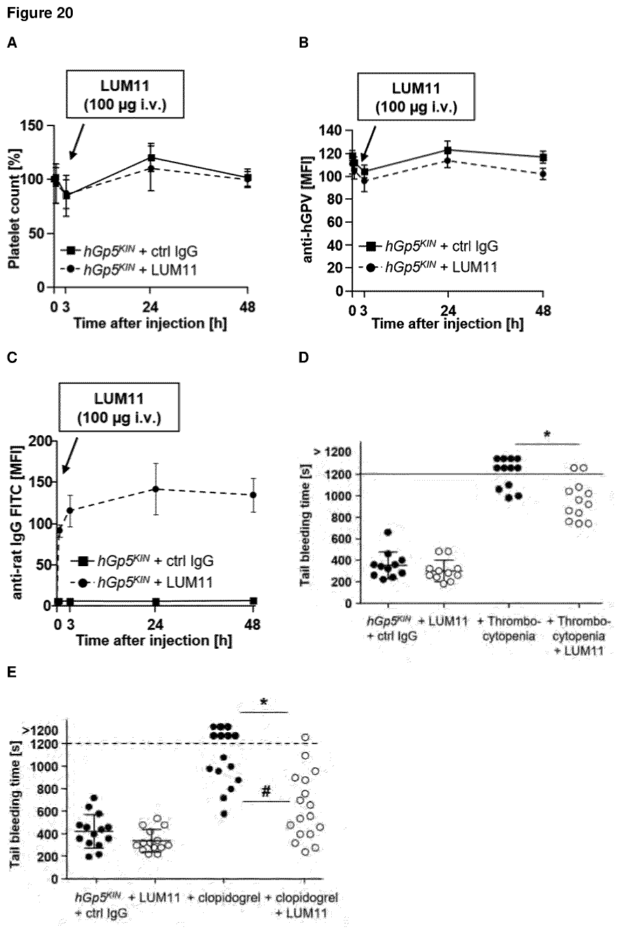

In vivo treatment of hGp5^{KIN} mice with LUM11 (i.v. injection) had no effect on platelet counts (FIG. 20A), in contrast to previously published anti-hGPV antibodies (Vollenberg et al.⁶⁶). LUM11 binds to the extracellular domain of human GPV without affecting GPV surface expression levels (FIG. 3B, C).

Thrombocytopenia is a major clinical challenge occurring frequently in the context of a variety of pathologies or medical treatments that is associated with increased bleeding and often with the need of immediate therapeutic intervention 44,45 To test a possible benefit of a GPV cleavage blockade in this setting, the present inventors induced severe thrombocytopenia by reducing platelet counts to 5-10% of normal by injecting a platelet-depleting antibody. ⁴⁶ While a resulting severe bleeding defect was observed in all platelet-depleted control mice, this was significantly attenuated by LUM11-treatment, and remarkably 10/12 LUM11-treated thrombocytopenic hGp5^{KIN} mice managed to stop bleeding within the observation period (FIG. 20D). The current clinical standard of care to reduce the risk of heart attack and ischaemic stroke is the pharmacological inhibition of platelet function by P2Y₁₂ ADP receptor blockers alone or in combination with acetyl salicylic acid (ASA). As seen in humans, hGp5^{KIN} mice treated with the P2Y₁₂-blocker clopidogrel exhibit increased bleeding that was reversed by treatment with LUM11 blocking thrombin-dependent GPV release from platelets (FIG. 20E). Thus, specific targeting of GPV with LUM11 prevented the prolongation of the bleeding time caused by thrombocytopenia and anti-platelet therapy, indicating clinical potential of anti-GPV treatment to restore haemostasis by improving thrombin-dependent fibrin formation. Of note, LUM11 accelerated thrombus formation upon FeCl₃-induced injury of mesenteric arterioles in hGp5^{KIN} mice (FIG. 21).

### REFERENCES

1 Swieringa, F., Spronk, H. M. H., Heemskerk, J. W. M. & van der Meijden, P. E. J. Integrating platelet and coagulation activation in fibrin clot formation. Res Pract Thromb Haemost 2(3), 450-460, doi:10.1002/rth2.12107 (2018).
2 Furie, B. & Furie, B. C. The molecular basis of blood coagulation. Cell 53(4), 505-518, doi:10.1016/0092-8674(88)90567-3 (1988).
3 Tomaiuolo, M., Brass, L. F. & Stalker, T. J. Regulation of platelet activation and coagulation and its role in vascular injury and arterial thrombosis. Interv Cardiol Clin 6(1), 1-12, doi:10.1016/j.iccl.2016.08.001 (2017).
4 Kahn, M. L. et al. A dual thrombin receptor system for platelet activation. Nature 394(6694), 690-694, doi:10.1038/29325 (1998).
5 Stalker, T. J. et al. Hierarchical organization in the hemostatic response and its relationship to the platelet-signaling network. Blood 121(10), 1875-1885, doi:10.1182/blood-2012-09-457739 (2013).
6 Versteeg, H. H., Heemskerk, J. W., Levi, M. & Reitsma, P. H. New fundamentals in hemostasis. Physiol. Rev. 93(1), 327-358, doi:10.1152/physrev.00016.2011 (2013).
7 Sang, Y., Roest, M., de Laat, B., de Groot, P. G. & Huskens, D. Interplay between platelets and coagulation. Blood Rev. 46, 100733, doi:10.1016/j.blre.2020.100733 (2021).
8 Silva, L. M. et al. Fibrin is a critical regulator of neutrophil effector function at the oral mucosal barrier. Science 374(6575), eabl5450, doi:10.1126/science.abl5450 (2021).
9 Ryu, J. K. et al. Fibrin-targeting immunotherapy protects against neuroinflammation and neurodegeneration. Nat. Immunol. 19, 1212-1223, doi:10.1038/s41590-018-0232-x (2018).
10 Mosnier, L. O., Zlokovic, B. V. & Griffin, J. H. The cytoprotective protein C pathway. Blood 109(8), 3161-3172, doi:10.1182/blood-2006-09-003004 (2007).
11 Ruggeri, Z. M. et al. Unravelling the mechanism and significance of thrombin binding to platelet glycoprotein Ib. Thromb. Haemost. 104(5), 894-902, doi:10.1160/TH10-09-0578 (2010).
12 Kahn, M. L. et al. Glycoprotein V-deficient platelets have undiminished thrombin responsiveness and do not exhibit a Bernard-Soulier phenotype. Blood 94(12), 4112-4121 (1999).
13 Li, R. & Emsley, J. The organizing principle of the platelet glycoprotein Ib-IX-V complex. J. Thromb. Haemost. 11(4), 605-614, doi:10.1111/jth.12144 (2013).
14 Mo, X., Liu, L., Lopez, J. A. & Li, R. Transmembrane domains are critical to the interaction between platelet glycoprotein V and glycoprotein Ib-IX complex. J. Thromb. Haemost. 10(9), 1875-1886, doi:10.1111/j.1538-7836.2012.04841.x (2012).
15 Moog, S. et al. Platelet glycoprotein V binds to collagen and participates in platelet adhesion and aggregation. Blood 98(4), 1038-1046 (2001).
16 Nieswandt, B. & Watson, S. P. Platelet-collagen interaction: is GPVI the central receptor? Blood 102(2), 449-461, doi:10.1182/blood-2002-12-3882 (2003).
17 Clemetson, K. J. A short history of platelet glycoprotein Ib complex. Thromb. Haemost. 98(1), 63-68 (2007).
18 Katsutani, S. et al. Cloning and characterization of the gene encoding the murine glycoprotein V: the conserved thrombin-cleavable protein on platelet surface. Thromb. Res. 92(1), 43-51, doi:10.1016/s0049-3848(98)00111-x (1998).
19 Ravanat, C. et al. Gene cloning of rat and mouse platelet glycoprotein V: identification of megakaryocyte-specific promoters and demonstration of functional thrombin cleavage. Blood 89(9), 3253-3262 (1997).
20 Ni, H. Increased thrombogenesis and embolus formation in mice lacking glycoprotein V. Blood 98(2), 368-373, doi:10.1182/blood.V98.2.368 (2001).
21 Rabie, T., Strehl, A., Ludwig, A. & Nieswandt, B. Evidence for a role of ADAM17 (TACE) in the regulation of platelet glycoprotein V. J. Biol. Chem. 280(15), 14462-14468, doi:10.1074/jbc.M500041200 (2005).
22 Ramakrishnan, V. et al. Increased thrombin responsiveness in platelets from mice lacking glycoprotein V. Proc. Natl. Acad. Sci. U.S.A 96(23), 13336-13341 (1999).
23 Nonne, C., Hechler, B., Cazenave, J. P., Gachet, C. & Lanza, F. Reassessment of in vivo thrombus formation in glycoprotein V deficient mice backcrossed on a C57Bl/6 strain. J Thromb. Haemost. 6(1), 210-212, doi:10.1111/j.1538-7836.2007.02825.x (2008).
24 McGowan, E. B., Ding, A. & Detwiler, T. C. Correlation of thrombin-induced glycoprotein V hydrolysis and platelet activation. J Biol. Chem. 258(18), 11243-11248 (1983).
25 Estevez, B. et al. Signaling-mediated cooperativity between glycoprotein Ib-IX and protease-activated receptors in thrombin-induced platelet activation. Blood 127(5), 626-636, doi:10.1182/blood-2015-04-638387 (2016).
26 Celikel, R. et al. Modulation of α-thrombin function by distinct interactions with platelet glycoprotein Iba. Science 301(5630), 218-221, doi:10.1126/science.1084183 (2003).
27 Dumas, J. J., Kumar, R., Seehra, J., Somers, W. S. & Mosyak, L. Crystal structure of the GpIbalpha-thrombin complex essential for platelet aggregation. Science 301(5630), 222-226, doi:10.1126/science.1083917 (2003).
28 Massberg, S. et al. A crucial role of glycoprotein VI for platelet recruitment to the injured arterial wall in vivo. J Exp. Med. 197(1), 41-49 (2003).
29 Stegner, D. et al. Foudroyant cerebral venous (sinus) thrombosis triggered through CLEC-2 and GPIIb/IIIa dependent platelet activation. Nature Cardiovascular Research 1(2), 132-141, doi:10.1038/s44161-021-00017-1 (2022).
30 Bender, M. et al. Combined in vivo depletion of glycoprotein VI and C-type lectin-like receptor 2 severely compromises hemostasis and abrogates arterial thrombosis in mice. Arterioscler. Thromb. Vasc. Biol. 33(5), 926-934, doi:10.1161/ATVBAHA.112.300672 (2013).
31 Nieswandt, B. et al. Long-term antithrombotic protection by in vivo depletion of platelet glycoprotein VI in mice. J Exp. Med. 193(4), 459-469, doi:10.1084/jem.193.4.459 (2001).
32 Mangin, P. et al. Thrombin overcomes the thrombosis defect associated with platelet GPVI/FcRγ deficiency. Blood 107(11), 4346-4353, doi:10.1182/blood-2005-10-4244 (2006).
33 Mangin, P. H. et al. Immobilized fibrinogen activates human platelets through glycoprotein VI. Haematologica 103(5), 898-907, doi:10.3324/haematol.2017.182972 (2018).
34 de Witt, S. M. et al. Identification of platelet function defects by multi-parameter assessment of thrombus formation. Nat Commun 5, 4257, doi:10.1038/ncomms5257 (2014).
35 Nagy, M. et al. Comparative analysis of microfluidics thrombus formation in multiple genetically modified mice: link to thrombosis and hemostasis. Front Cardiovasc Med 6, 99, doi:10.3389/fcvm.2019.00099 (2019).
36 Fredenburgh, J. C., Stafford, A. R., Leslie, B. A. & Weitz, J. I. Bivalent binding to gammaA/gamma′-fibrin engages both exosites of thrombin and protects it from inhibition by the antithrombin-heparin complex. J. Biol. Chem. 283(5), 2470-2477, doi:10.1074/jbc.M707710200 (2008).
37 Stoll, G. & Nieswandt, B. Thrombo-inflammation in acute ischaemic stroke—implications for treatment. Nat. Rev. Neurol. 15(8), 473-481, doi:10.1038/s41582-019-0221-1 (2019).
38 Petrera, N. S. et al. Long range communication between exosites 1 and 2 modulates thrombin function. J Biol. Chem. 284(38), 25620-25629, doi:10.1074/jbc.M109.000042 (2009).
39 Bock, L. C., Griffin, L. C., Latham, J. A., Vermaas, E. H. & Toole, J. J. Selection of single-stranded DNA molecules that bind and inhibit human thrombin. Nature 355(6360), 564-566, doi:10.1038/355564a0 (1992).
40 Tasset, D. M., Kubik, M. F. & Steiner, W. Oligonucleotide inhibitors of human thrombin that bind distinct epitopes. J Mol. Biol. 272(5), 688-698, doi:10.1006/jmbi.1997.1275 (1997).
41 Griner, S. et al. Anti-glycoprotein VI treatment severely compromises hemostasis in mice with reduced u201 levels or concomitant aspirin therapy. Circulation 110(18), 2946-2951, doi:10.1161/01.CIR.0000146341.63677.3C (2004).
42 Pleines, I. et al. Megakaryocyte-specific RhoA deficiency causes macrothrombocytopenia and defective platelet activation in hemostasis and thrombosis. Blood 119(4), 1054-1063, doi:10.1182/blood-2011-08-372193 (2012).
43 Deppermann, C. et al. Gray platelet syndrome and defective thrombo-inflammation in Nbeal2-deficient mice. J Clin. Invest. 123(8), 3331-3342, doi:10.1172/JCI69210 (2013).
44 Bakchoul, T. & Marini, I. Drug-associated thrombocytopenia. Hematology Am. Soc. Hematol. Educ. Program 2018, 576-583, doi:10.1182/asheducation-2018.1.576 (2018).
45 Aster, R. H. & Bougie, D. W. Drug-induced immune thrombocytopenia. N. Engl. J. Med 357(6), 580-587, doi:10.1056/NEJMra066469 (2007).
46 Morowski, M. et al. Only severe thrombocytopenia results in bleeding and defective thrombus formation in mice. Blood 121(24), 4938-4947, doi:10.1182/blood-2012-10-461459 (2013).
47 Sekhon, U. D. S. et al. Platelet-mimicking procoagulant nanoparticles augment hemostasis in animal models of bleeding. Sci. Transl. Med. 14, eabb8975, doi:10.1126/scitranslmed.abb8975 (2022).
48 Mast, A. E. & Ruf, W. Regulation of coagulation by tissue factor pathway inhibitor: Implications for haemophilia therapy. J Thromb. Haemost. 20(6), 1290-1300, doi:10.1111/jth.15697 (2022).
49 Holtkotter, O. et al. Integrin alpha 2-deficient mice develop normally, are fertile, but display partially defective platelet interaction with collagen. J. Biol. Chem. 277(13), 10789-10794, doi:10.1074/jbc.M112307200 (2002).
50 Jackson, B. et al. RhoA is dispensable for skin development, but crucial for contraction and directed migration of keratinocytes. Mol. Biol. Cell 22(5), 593-605, doi:10.1091/mbc.E09-10-0859 (2011).
51 Knight, C. G. et al. Collagen-platelet interaction: Gly-Pro-Hyp is uniquely specific for platelet Gp VI and mediates platelet activation by collagen. Cardiovasc. Res. 41(2), 450-457, doi:Doi 10.1016/S0008-6363(98)00306-X (1999).
52 Schulte, V. et al. Targeting of the collagen-binding site on glycoprotein VI is not essential for in vivo depletion of the receptor. Blood 101(10), 3948-3952, doi:10.1182/blood-2002-10-3242 (2003).
53 Bergmeier, W. et al. Flow cytometric detection of activated mouse integrin alphaIIbbeta3 with a novel monoclonal antibody. Cytometry 48(2), 80-86, doi:10.1002/cyto.10114 (2002).
54 Stegner, D. et al. Thrombopoiesis is spatially regulated by the bone marrow vasculature. Nat Commun 8, 127, doi:10.1038/s41467-017-00201-7 (2017).
55 Nieswandt, B., Bergmeier, W., Rackebrandt, K., Gessner, J. E. & Zirngibl, H. Identification of critical antigen-specific mechanisms in the development of immune thrombocytopenic purpura in mice. Blood 96(7), 2520-2527 (2000).
56 Shida, Y. et al. Analysis of the role of von Willebrand factor, platelet glycoprotein VI-, and α2β1-mediated collagen binding in thrombus formation. Blood 124(11), 1799-1807, doi:10.1182/blood-2013-09-521484 (2014).
57 Nieswandt, B. et al. Acute systemic reaction and lung alterations induced by an antiplatelet integrin GPIIb/IIIa antibody in mice. Blood 94(2), 684-693 (1999).
58 Hemker, H. C. & Beguin, S. Thrombin generation in plasma: its assessment via the endogenous thrombin potential. Thromb. Haemost. 74(1), 134-138 (1995).
59 Hemker, H. C. et al. Calibrated automated thrombin generation measurement in clotting plasma. Pathophysiol. Haemost. Thromb. 33(1), 4-15, doi:10.1159/000071636 (2003).
60 Brouns, S. L. N. et al. Platelet-primed interactions of coagulation and anticoagulation pathways in flow-dependent thrombus formation. Sci. Rep. 10, 11910, doi:10.1038/s41598-020-68438-9 (2020).
61 van der Walt, S. et al. scikit-image: image processing in Python. PeerJ 2, e453, doi:10.7717/peerj.453 (2014).
62 Schindelin, J. et al. Fiji: an open-source platform for biological-image analysis. Nat. Methods 9(7), 676-682, doi:10.1038/nmeth.2019 (2012).
63 Amich, J. et al. Three-dimensional light sheet fluorescence microscopy of lungs to dissect local host immune-Aspergillus fumigatus interactions. mBio 11(1), doi:10.1128/mBio.02752-19 (2020).
64 Kleinschnitz, C. et al. Targeting platelets in acute experimental stroke: impact of glycoprotein Ib, VI, and IIb/IIIa blockade on infarct size, functional outcome, and intracranial bleeding. Circulation 115(17), 2323-2330, doi:10.1161/CIRCULATIONAHA.107.691279 (2007).
65 Carstairs, K. C. The identification of platelets and platelet antigens in histological sections. J. Pathol. Bacteriol. 90(1), 225-231, doi:10.1002/path.1700900124 (1965).
66 Vollenberg, R. et al. Glycoprotein V is a relevant immune target in patients with immune thrombocytopenia. Haematologica 104(6), 1237-1243, doi:10.3324/haematol.2018.211086 (2019).
67 Wahl et al. Improved Radioimaging and Tumor Localization with Monoclonal F(ab′)2 J. Nucl. Med. 1983; 24(4): 316-325
68 Harlow and Lane, “Antibodies, A Laboratory Manual” CSH Press 1988, Cold Spring Harbor N.Y.
69 Morrison. Transfectomas Provide Novel Chimeric Antibodies Science. 1985; 229(4719):1202-7
70 Oi et al. Chimeric Antibodies BioTechniques. 1986; 4(3): 214-221
71 Gillies et al. High-level expression of chimeric antibodies using adapted cDNA variable region cassettes J. Immunol. Methods 1989; 125(1-2): 191-202;
72 Riechmann et al. Reshaping human antibodies for therapy. Nature 1988. 332(6162): 323-7
73 Padlan. A possible procedure for reducing the immunogenicity of antibody variable domains while preserving their ligand-binding properties. Mol Immunol. 1991; 28(4-5): 489-498
74 Studnicka et al. Human-engineered monoclonal antibodies retain full specific binding activity by preserving non-CDR complementarity modulating residues. Prot Eng. 1994; 7(6): 805-814
75 Roguska et al. Humanization of murine monoclonal antibodies through variable domain resurfacing. PNAS 1994; 91(3): 969-973
76 Jespers et al. Guiding the Selection of Human Antibodies from Phage Display Repertories to a Single Epitope of an Antigen. Nature Biotechnology. 1994; 12(9): 899-903
77 Canfield and Morrison. The Binding Affinity of Human IgG for its High Affinity Fc Receptor Is Determined by Multiple Amino Acids in the CH2 Domain and Is Modulated by the Hinge Region. J Exp Med. 1991; 173(6): 1483-1491
78 Lund et al. Human Fc gamma RI and FC gamma RII interact with distinct but overlapping sites on human IgG. J Immunol. 1991; 147(8): 2657-2662
79 Hellstrom et al., Antibodies for Drug Delivery. Controlled Drug Delivery, 2nd Ed., 623-53 (Robinson et al., eds., 1987)
80 Thorpe et al. The Preparation and Cytotoxic Properties of Antibody-Toxin Conjugates. Immunol Rev. 1982; 62(1): 119-58
81 Dubowchik et al. Receptor-mediated and enzyme-dependent targeting of cytotoxic anticancer drugs. Pharmacology and Therapeutics 1999; 83(2): 67-123
82 Poly(ethyleneglycol) Chemistry, Biotechnical and Biomedical Applications, (J. Milton Harris (ed.), Plenum Press, New York, 1992)
83 Poly(ethyleneglycol) Chemistry and Biological Applications, (J. Milton Harris and S. Zalipsky, eds., American Chemical Society, Washington D. C, 1997)
84 Bioconjugation Protein Coupling Techniques for the Biomedical Sciences, (M. Aslam and A. Dent, eds., Grove Publishers, New York, 1998)
85 Chapman. PEGylated antibodies and antibody fragments for improved therapy: a review. Advanced Drug Delivery Reviews 2002; 54(4): 531-545
86 Azorsa D O, et al. Measurement of GPV Released by Activated Platelets Using a Sensitive Immunocapture ELISA—Its Use to Follow Platelet Storage in Transfusion. Thrombosis and Haemostasis. 1999; 81(1): 131-8
87 Moog S, et al. Platelet glycoprotein V binds to collagen and participates in platelet adhesion and aggregation Blood. 2001; 98(4): 1038-46
88 Tatusova et al. BLAST 2 SEQUENCES, a new tool for comparing protein and nucleotide sequences. FEMS Microbiol. Lett. 1999; 174: 247-250

1-4. (canceled)

5. An antibody, or a functional fragment or derivative thereof, comprising:
(i) a VH domain comprising a CDR1 having an amino acid sequence as shown in SEQ ID NO:15, a CDR2 having an amino acid sequence as shown in SEQ ID NO:16, and a CDR3 having an amino acid sequence as shown in SEQ ID NO:17, and a VL domain comprising a CDR1 having an amino acid sequence in accordance with the amino acid sequence as shown in SEQ ID NO:18, a CDR2 having an amino acid sequence in accordance with the amino acid sequence as shown in SEQ ID NO:19, and a CDR3 having an amino acid sequence in accordance with the amino acid sequence as shown in SEQ ID NO:20;
(ii) a VH domain comprising a CDR1 having an amino acid sequence as shown in SEQ ID NO:1, a CDR2 having an amino acid sequence as shown in SEQ ID NO:2, and a CDR3 having an amino acid sequence as shown in SEQ ID NO:3, and a VL domain comprising a CDR1 having an amino acid sequence in accordance with the amino acid sequence as shown in SEQ ID NO:4, a CDR2 having an amino acid sequence in accordance with the amino acid sequence as shown in SEQ ID NO:5, and a CDR3 having an amino acid sequence in accordance with the amino acid sequence as shown in SEQ ID NO:6.

6. The antibody, fragment, or derivative according to claim 5, wherein:
(i) the VH domain comprises an amino acid sequence as shown in SEQ ID NO:21, and the VL domain comprises an amino acid sequence as shown in SEQ ID NO:23; or
(ii) the VH domain comprises an amino acid sequence as shown in SEQ ID NO:7, and the VL domain comprises an amino acid sequence as shown in SEQ ID NO:9.

7-8. (canceled)

9. A nucleic acid encoding the antibody, fragment, or derivative according to claim 5.

10. A pharmaceutical composition comprising the antibody, fragment, or derivative according to claim 5.

11-15. (canceled)

16. A method of treating a haemorrhagic condition in a subject in need thereof, comprising administering to the subject an effective amount of:
(i) an antibody, fragment, or derivative according to claim 5;
(ii) a nucleic acid encoding the antibody, fragment, or derivative of (i), or
(iii) a pharmaceutical composition comprising the antibody, fragment, or derivative of (i) or the nucleic acid of (ii).

17. The method of claim 16, wherein:
(i) the haemorrhagic condition is caused by a platelet disorder; and/or
(ii) the haemorrhagic condition is chosen from inflammatory bleeding, haemophilia/FVIII, bleeding due to anti-platelet therapy, bleeding due to anti-coagulant therapy, haemorrhagic stroke, excessive bleeding due to sepsis, excessive bleeding due to thrombocytopenia, excessive bleeding due to disseminated intravascular coagulation (DIC), excessive bleeding due to chemotherapy, excessive bleeding due to haemolytic-uremic syndrome, excessive bleeding upon administration of soluble GPV, and excessive bleeding due to HIV infection.

18. The method of claim 16, further comprising administering to the subject an effective amount of a coagulant other than said antibody, fragment, derivative, nucleic acid, or pharmaceutical composition.

19. The method of claim 18, wherein the coagulant other than said antibody, fragment, derivative, nucleic acid, or pharmaceutical composition is chosen from an anti-fibrinolytic agent, a platelet concentrate, a coagulation factor concentrate, and fresh frozen plasma.

20. A pharmaceutical composition comprising the nucleic acid according to claim 9.

21. The pharmaceutical composition of claim 10, further comprising a pharmaceutically acceptable excipient.


SEQUENCE LISTING (38 sequences)

SEQ ID NO: 1 - protein, 5 aa, unidentified
DYNMA

SEQ ID NO: 2 - protein, 17 aa, unidentified
TFIYDGSNTYYGDSVKG

SEQ ID NO: 3 - protein, 9 aa, unidentified
HTWANWFAY

SEQ ID NO: 4 - protein, 11 aa, unidentified
RGSQNINNYLA

SEQ ID NO: 5 - protein, 7 aa, unidentified
STNSLQT

SEQ ID NO: 6 - protein, 8 aa, unidentified
YQYNNGYT

SEQ ID NO: 7 - protein, 118 aa, unidentified
EVQLVESGGGLVQPGRSLKLSCAASGFTFSDYNMAWVRQAPKKGLEWVATFIYDGSNTYYGDSVKGRFTVSRDNAKSTLYLQMDSLRSEDTATYYCTTHTWANWFAYWGQGTLVTVSS

SEQ ID NO: 8 - protein, 137 aa, unidentified
  SIGNAL 1..19
MDIRLSLVFLVLFIKGVQCEVQLVESGGGLVQPGRSLKLSCAASGFTFSDYNMAWVRQAPKKGLEWVATFIYDGSNTYYGDSVKGRFTVSRDNAKSTLYLQMDSLRSEDTATYYCTTHTWANWFAYWGQGTLVTVSS

SEQ ID NO: 9 - protein, 106 aa, unidentified
NIQLTQSPALLSASVGDRVTLSCRGSQNINNYLAWYQQKLGEAPKLLIYSTNSLQTGIPSRFSGSGSGTDYTLTISSLQPEDVATYFCYQYNNGYTFGPGTKLELK

SEQ ID NO: 10 - protein, 126 aa, unidentified
  SIGNAL 1..20
MMAPVQLLVLLLLWLPAMRCNIQLTQSPALLSASVGDRVTLSCRGSQNINNYLAWYQQKLGEAPKLLIYSTNSLQTGIPSRFSGSGSGTDYTLTISSLQPEDVATYFCYQYNNGYTFGPGTKLELK

SEQ ID NO: 11 - DNA, 354 bp, unidentified
  source 1..354: mol_type = unassigned DNA
gaggtgcagctggtggagtctgggggcggcttagtgcagcctggaaggtccctgaaactctcctgtgcagcctcaggattcactttcagtgactataacatggcctgggtccgccaggctccaaagaagggactggagtgggtcgcaacctttatttatgatggtagtaacacttactatggagactccgtgaagggccgattcactgtctccagagataatgcaaaaagcaccctatacctgcaaatggacagtctgaggtctgaggacacggccacttattactgtacaacacacacctgggccaattggtttgcttactggggccaaggcactctggtcactgtctcttca

SEQ ID NO: 12 - DNA, 411 bp, unidentified
  source 1..411: mol_type = unassigned DNA
  sig_peptide 1..57
atggacatcaggctcagcttggttttccttgtccttttcataaaaggtgtccagtgtgaggtgcagctggtggagtctgggggcggcttagtgcagcctggaaggtccctgaaactctcctgtgcagcctcaggattcactttcagtgactataacatggcctgggtccgccaggctccaaagaagggactggagtgggtcgcaacctttatttatgatggtagtaacacttactatggagactccgtgaagggccgattcactgtctccagagataatgcaaaaagcaccctatacctgcaaatggacagtctgaggtctgaggacacggccacttattactgtacaacacacacctgggccaattggtttgcttactggggccaaggcactctggtcactgtctcttca

SEQ ID NO: 13 - DNA, 318 bp, unidentified
  source 1..318: mol_type = unassigned DNA
aacatccaactgacccagtctcctgcactcctgtctgcatctgtgggagacagagtcactcttagctgcagaggaagtcagaatattaacaattacttagcctggtaccaacaaaagcttggagaagctcccaaactcctgatatatagtacaaacagtttgcaaacgggcatcccatcaaggttcagtggcagtggatctgggacagattacacactcaccatcagcagcctgcagcctgaagatgttgccacatatttctgctatcagtataacaacgggtacacgtttggacctgggaccaagctggaactgaaa

SEQ ID NO: 14 - DNA, 378 bp, unidentified
  source 1..378: mol_type = unassigned DNA
  sig_peptide 1..60
atgatggcaccagttcaactcttagtgctgctgctgctctggctcccagccatgagatgtaacatccaactgacccagtctcctgcactcctgtctgcatctgtgggagacagagtcactcttagctgcagaggaagtcagaatattaacaattacttagcctggtaccaacaaaagcttggagaagctcccaaactcctgatatatagtacaaacagtttgcaaacgggcatcccatcaaggttcagtggcagtggatctgggacagattacacactcaccatcagcagcctgcagcctgaagatgttgccacatatttctgctatcagtataacaacgggtacacgtttggacctgggaccaagctggaactgaaa

SEQ ID NO: 15 - protein, 5 aa, unidentified
SYGVN

SEQ ID NO: 16 - protein, 16 aa, unidentified
AISSGGSTYYNSSLKS

SEQ ID NO: 17 - protein, 12 aa, unidentified
GYYGYDYDYFDY

SEQ ID NO: 18 - protein, 11 aa, unidentified
KGSQNINNYLA

SEQ ID NO: 19 - protein, 7 aa, unidentified
NTNSLQT

SEQ ID NO: 20 - protein, 8 aa, unidentified
YQYNNGYT

SEQ ID NO: 21 - protein, 120 aa, unidentified
QVQLKESGPGLVQPSQTLSLTCTVSGFSLTSYGVNWVRQPPGKGLEWIAAISSGGSTYYNSSLKSRLSISRDTSKSQVFLKMNSLQTEDTAIYFCTRGYYGYDYDYFDYWGQGVMVTVSS

SEQ ID NO: 22 - protein, 139 aa, unidentified
  SIGNAL 1..19
MAVLVLLLCLVTFPNCVLSQVQLKESGPGLVQPSQTLSLTCTVSGFSLTSYGVNWVRQPPGKGLEWIAAISSGGSTYYNSSLKSRLSISRDTSKSQVFLKMNSLQTEDTAIYFCTRGYYGYDYDYFDYWGQGVMVTVSS

SEQ ID NO: 23 - protein, 106 aa, unidentified
NIQLTQSPSLLSASVGDRVTLSCKGSQNINNYLAWYQQKLGEAPKLLIYNTNSLQTGIPSRFSGGGSGADYTLTISSLQPEDVATYLCYQYNNGYTFGAGTKMELK

SEQ ID NO: 24 - protein, 125 aa, unidentified
  SIGNAL 1..19
MAPVQLLVLLLLWLPAMRCNIQLTQSPSLLSASVGDRVTLSCKGSQNINNYLAWYQQKLGEAPKLLIYNTNSLQTGIPSRFSGGGSGADYTLTISSLQPEDVATYLCYQYNNGYTFGAGTKMELK

SEQ ID NO: 25 - DNA, 360 bp, unidentified
  source 1..360: mol_type = unassigned DNA
caggtgcagctgaaagagtcaggacctggtctggtgcagccctcacagaccctgtctctcacctgcactgtctctggattctcattaaccagttatggtgtaaactgggttcgccagcctccagggaagggtctggagtggattgcagcaatatcaagtggtggaagcacatattataattcatctctcaaatcccgactgagcatcagcagggacacctccaagagccaagttttcttaaaaatgaacagtctgcaaactgaagacacagccatttacttctgtaccaggggatactacgggtatgactacgactactttgattattggggccaaggagtcatggtcacagtctcctca

SEQ ID NO: 26 - DNA, 417 bp, unidentified
  source 1..417: mol_type = unassigned DNA
  sig_peptide 1..57
atggctgtcctggtgctgttgctctgcctggtgacattcccaaactgtgtcctgtcccaggtgcagctgaaagagtcaggacctggtctggtgcagccctcacagaccctgtctctcacctgcactgtctctggattctcattaaccagttatggtgtaaactgggttcgccagcctccagggaagggtctggagtggattgcagcaatatcaagtggtggaagcacatattataattcatctctcaaatcccgactgagcatcagcagggacacctccaagagccaagttttcttaaaaatgaacagtctgcaaactgaagacacagccatttacttctgtaccaggggatactacgggtatgactacgactactttgattattggggccaaggagtcatggtcacagtctcctca

SEQ ID NO: 27 - DNA, 318 bp, unidentified
  source 1..318: mol_type = unassigned DNA
aacatccagctgacccagtctccttcactcctgtctgcatctgtgggagacagagtcactcttagctgcaaaggaagtcagaatattaacaattacttagcctggtaccaacaaaagcttggagaagctcccaaactcctgatatataatacaaacagtttgcaaacgggcatcccatcaaggttcagtggcggtggatctggtgcagattacacactcaccatcagcagcctgcagcctgaagatgttgccacatatctctgctatcagtataacaacgggtacacgtttggagctgggaccaagatggaactgaaa

SEQ ID NO: 28 - DNA, 375 bp, unidentified
  source 1..375: mol_type = unassigned DNA
  sig_peptide 1..57
atggcaccagttcaactcttagtgctgctgctgctctggctcccagccatgagatgtaacatccagctgacccagtctccttcactcctgtctgcatctgtgggagacagagtcactcttagctgcaaaggaagtcagaatattaacaattacttagcctggtaccaacaaaagcttggagaagctcccaaactcctgatatataatacaaacagtttgcaaacgggcatcccatcaaggttcagtggcggtggatctggtgcagattacacactcaccatcagcagcctgcagcctgaagatgttgccacatatctctgctatcagtataacaacgggtacacgtttggagctgggaccaagatggaactgaaa

SEQ ID NO: 29 - DNA, 3493 bp, Homo sapiens
  source 1..3493: mol_type = genomic DNA
  CDS 32..1714
agttactttggagtgcagaaccatttcagacatgctgagggggactctactgtgcgcggtgctcgggcttctgcgcgcccagcccttcccctgtccgccagcttgcaagtgtgtcttccgggacgccgcgcagtgctcggggggcgacgtggcgcgcatctccgcgctaggcctgcccaccaacctcacgcacatcctgctcttcggaatgggccgcggcgtcctgcagagccagagcttcagcggcatgaccgtcctgcagcgcctcatgatctccgacagccacatttccgccgttgcccccggcaccttcagtgacctgataaaactgaaaaccctgaggctgtcgcgcaacaaaatcacgcatcttccaggtgcgctgctggataagatggtgctcctggagcagttgtttttggaccacaatgcgctaaggggcattgaccaaaacatgtttcagaaactggttaacctgcaggagctcgctctgaaccagaatcagctcgatttccttcctgccagtctcttcacgaatctggagaacctgaagttgttggatttatcgggaaacaacctgacccacctgcccaaggggttgcttggagcacaggctaagctcgagagacttctgctccactcgaaccgccttgtgtctctggattcggggctgttgaacagcctgggcgccctgacggagctgcagttccaccgaaatcacatccgttccatcgcacccggggccttcgaccggctcccaaacctcagttctttgacgctttcgagaaaccaccttgcgtttctcccctctgcgctctttcttcattcgcacaatctgactctgttgactctgttcgagaacccgctggcagagctcccgggggtgctcttcggggagatggggggcctgcaggagctgtggctgaaccgcacccagctgcgcaccctgcccgccgccgccttccgcaacctgagccgcctgcggtacttaggggtgactctgagcccgcggctgagcgcgcttccgcagggcgccttccagggccttggcgagctccaggtgctcgccctgcactccaacggcctgaccgccctccccgacggcttgctgcgcggcctcggcaagctgcgccaggtgtccctgcgccgcaacaggctgcgcgccctgccccgtgccctcttccgcaatctcagcagcctggagagcgtccagctcgaccacaaccagctggagaccctgcctggcgacgtgtttggggctctgccccggctgacggaggtcctgttggggcacaactcctggcgctgcgactgtggcctggggcccttcctggggtggctgcggcagcacctaggcctcgtgggcggggaagagcccccacggtgcgcaggccctggggcgcacgccggcctgccgctctgggccctgccggggggtgacgcggagtgcccgggcccccggggcccgcctccccgccccgctgcggacagctcctcggaagcccctgtccacccagccttggctcccaacagctcagaaccctgggtgtgggcccagccggtgaccacgggcaaaggtcaagatcatagtccgttctgggggttttattttctgcttttagctgttcaggccatgatcaccgtgatcatcgtgtttgctatgattaaaattggccaactctttcgaaaattaatcagagagagagcccttgggtaaaccaatgggaaaatcttctaattacttagaacctgaccagatgtggctcggaggggaatccagacccgctgctgtcttgctctccctcccctccccactcctcctctcttcttcctcttctctctcactgccacgccttcctttccctcctcctccccctctccgctctgtgctcttcattctcacaggcccgcaacccctcctctctgtgtcccccgcccgttcctggaaactgagcttgacgtttgtaaactgtggttgcctgccttccccagctcccacgcgggtgtgcgctgacactgccgggggcgctggactgtgttggacccatccgtgctccgctgtgcctggcttggcgtctggtggagagaggggcctcttcagtgtctactgagtaaggggacagctccaggccggggcctgtctcctgcacagagtaagccggtaaatgtttgtgaaatcaatgcgtggataaaggaacacatgccatccaagtgatgatggcttttcctggagggaaaggataggctgttgctctatctaattttttgtttttgtttttggacagtctagctctgtggcccaggctggcgtgcagtgggccgtctcagttcactgcagcctccgcctcccaggttcaagtgattctcatgcctcagcgttctgagtagctgggattagaggcgtgtgccactacacccggctaatttttgtactttttaaagtagagacggggctttgccatattggcctggctgatctcaaactcctggtcttgaactcctggccacaagtgatctgcccgccttggcctcccaaagtgctgggattacaggcgtaagccactacacctggccctcttcatcgaattttatttgagaagtagagctcttgccattttttcccttgctccatttttctcactttatgtctctctgacctatgggctacttgggagagcactggactccattcatgcatgagcattttcaggataagcgacttctgtgaggctgagagaggaagaaaacacggagccttccctccaggtgcccagtgtaggtccagcgtgtttcctgagcctcctgtgagtttccacttgctttacatccatgcaacatgtcattttgaaactggattgatttgcatttcctggaactctgccacctcatttcacaagcatttatggagcagttaacatgtgactggtattcatgaatataatgataagcttgattctagttcagctgctgtcacagtctcatttgttcttccaactgaaagccgtaaaacctttgttgctttaattgaatgtctgtgcttatgagaggcagtggttaaaacaggggctggcgagttgacaactgtgggttcaaatcccagctctaccacttactaactgcatgggactttgggtaagacacctgcttacattctctaagccttggtttcctgaaccttaaaacaggataacatagtacctgcttcgtagagtttttgtgagaattaaaggcaataaagcatataatgacttagcccagcggcctgcaggcaatacatgttaatgaatgttagctattattactaaaggatgagcaattattattggcatcatgatttctaaagaagagctttgagttggtatttttctctgtgtataagggtaagtccgaactttctcagactggaggttacattcacatcagtctgtcttcccctgcggatggcctcagccctgggtggccagactctgtgctcacaatccagagcaatggatcc

SEQ ID NO: 30 - protein, 560 aa, Homo sapiens
MLRGTLLCAVLGLLRAQPFPCPPACKCVFRDAAQCSGGDVARISALGLPTNLTHILLFGMGRGVLQSQSFSGMTVLQRLMISDSHISAVAPGTFSDLIKLKTLRLSRNKITHLPGALLDKMVLLEQLFLDHNALRGIDQNMFQKLVNLQELALNQNQLDFLPASLFTNLENLKLLDLSGNNLTHLPKGLLGAQAKLERLLLHSNRLVSLDSGLLNSLGALTELQFHRNHIRSIAPGAFDRLPNLSSLTLSRNHLAFLPSALFLHSHNLTLLTLFENPLAELPGVLFGEMGGLQELWLNRTQLRTLPAAAFRNLSRLRYLGVTLSPRLSALPQGAFQGLGELQVLALHSNGLTALPDGLLRGLGKLRQVSLRRNRLRALPRALFRNLSSLESVQLDHNQLETLPGDVFGALPRLTEVLLGHNSWRCDCGLGPFLGWLRQHLGLVGGEEPPRCAGPGAHAGLPLWALPGGDAECPGPRGPPPRPAADSSSEAPVHPALAPNSSEPWVWAQPVTTGKGQDHSPFWGFYFLLLAVQAMITVIIVFAMIKIGQLFRKLIRERALG

SEQ ID NO: 31 - protein, 544 aa, Homo sapiens
QPFPCPPACKCVFRDAAQCSGGDVARISALGLPTNLTHILLFGMGRGVLQSQSFSGMTVLQRLMISDSHISAVAPGTFSDLIKLKTLRLSRNKITHLPGALLDKMVLLEQLFLDHNALRGIDQNMFQKLVNLQELALNQNQLDFLPASLFTNLENLKLLDLSGNNLTHLPKGLLGAQAKLERLLLHSNRLVSLDSGLLNSLGALTELQFHRNHIRSIAPGAFDRLPNLSSLTLSRNHLAFLPSALFLHSHNLTLLTLFENPLAELPGVLFGEMGGLQELWLNRTQLRTLPAAAFRNLSRLRYLGVTLSPRLSALPQGAFQGLGELQVLALHSNGLTALPDGLLRGLGKLRQVSLRRNRLRALPRALFRNLSSLESVQLDHNQLETLPGDVFGALPRLTEVLLGHNSWRCDCGLGPFLGWLRQHLGLVGGEEPPRCAGPGAHAGLPLWALPGGDAECPGPRGPPPRPAADSSSEAPVHPALAPNSSEPWVWAQPVTTGKGQDHSPFWGFYFLLLAVQAMITVIIVFAMIKIGQLFRKLIRERALG

SEQ ID NO: 32 - DNA, 2215 bp, Mus musculus
  source 1..2215: mol_type = genomic DNA
  CDS 30..1733
tcagtccagggtgcagaactgcttcagacatgctaagaagcgccctgctgtccgcggtgctcgcactcttgcgtgcccaaccttttccctgccccaaaacctgcaagtgtgtggtccgcgatgccgcgcagtgctcgggcggcagcgtggctcacatcgctgagctaggtctgcctacgaacctcacacacatcctgctcttccgaatggaccagggcatattgcggaaccacagcttcagcggcatgacagtccttcagcgcctgatgctctcagatagccacatttccgccatcgaccccggcaccttcaatgacctggtaaaactgaaaaccctcaggttgacgcgcaacaaaatctctcgtcttccacgtgcgatcctggataagatggtactcttggaacagctgttcttggaccacaatgcactaagggaccttgatcaaaacctgtttcagcaactgcgtaaccttcaggagctcggtttgaaccagaatcagctctcttttcttcctgctaaccttttctcgagcctgagagaactgaagttgttggatttatcgcgaaacaacctgacccacctgcccaagggactgcttggggctcaagttaagcttgagaaactgctgctctattcaaaccagctcacgtctgtggattcggggctgctgagcaacctgggcgccctgactgagctgcggctggagcggaatcacctccgctccgtagccccgggtgccttcgaccgcctcggaaacctgagctccttgactctatccggaaacctcctggagtctctgccgcccgcgctcttccttcacgtgagcagcgtgtctcggctgactctgttcgagaaccccctggaggagctcccggacgtgttgttcggggagatggccggcctgcgggagctgtggctgaacggcacccacctgagcacgctgcccgccgctgccttccgcaacctgagcggcttgcagacgctggggctgacgcggaacccgcgcctgagcgcgctcccgcgcggcgtgttccagggcctacgggagctgcgcgtgctcgcgctgcacaccaacgccctggcggagctgcgggacgacgcgctgcgcggcctcgggcacctgcgccaggtgtcgctgcgccacaaccggctgcgggccctgccccgcacgctcttccgcaacctcagcagcctcgagagcgtgcagctagagcacaaccagctggagacgctgccaggagacgtgttcgcggctctgccccagctgacccaggtcctgctgggtcacaacccctggctctgcgactgtggcctgtggcccttcctccagtggctgcggcatcacccggacatcctgggccgagacgagcccccgcagtgccgtggcccggagccacgcgccagcctgtcgttctgggagctgctgcagggtgacccgtggtgcccggatcctcgcagcctgcctctcgaccctccaaccgaaaatgctctggaagccccggttccgtcctggctgcctaacagctggcagtcccagacgtgggcccagctggtggccaggggtgaaagtcccaataacaggctctactggggtctttatattctgcttctagtagcccaggccatcatagccgcgttcatcgtgtttgccatgattaaaatcggccagctgtttcgaacattaatcagagagaagctcttgttagaggcaatgggaaaatcgtgtaactaatgaaactgaccagagcattgtggacggggccccaaggagaatgcagtcaggatgctggcgtgccattacactatttcccaggccttttctcctctcccgtgctcttagtgtctcttcttctcccctctcttcagaagtagcttttgtaaatcgctactgctttctagcctggcctgggttacctcctctgctgttagtttcaagggggctgagggtgggggttcgacgggacttggctcatcaggtccaactgtgcagcgctgggtgcctagtggagagaggagccctttcttggtttctgaatttgaggacacatcctgccagtgggcaagacctctccgggacccagcaagggttgagtaacatttgctgaaggaacaccggcttaaaacgaaccctaggtccaagagatgaaggctcttcccaaaataaaggtggagtgttcttgtccctttacctgaaaggaaaaaaaaaaaaaaaaa

SEQ ID NO: 33 - protein, 567 aa, Mus musculus
MLRSALLSAVLALLRAQPFPCPKTCKCVVRDAAQCSGGSVAHIAELGLPTNLTHILLFRMDQGILRNHSFSGMTVLQRLMLSDSHISAIDPGTFNDLVKLKTLRLTRNKISRLPRAILDKMVLLEQLFLDHNALRDLDQNLFQQLRNLQELGLNQNQLSFLPANLFSSLRELKLLDLSRNNLTHLPKGLLGAQVKLEKLLLYSNQLTSVDSGLLSNLGALTELRLERNHLRSVAPGAFDRLGNLSSLTLSGNLLESLPPALFLHVSSVSRLTLFENPLEELPDVLFGEMAGLRELWLNGTHLSTLPAAAFRNLSGLQTLGLTRNPRLSALPRGVFQGLRELRVLALHTNALAELRDDALRGLGHLRQVSLRHNRLRALPRTLFRNLSSLESVQLEHNQLETLPGDVFAALPQLTQVLLGHNPWLCDCGLWPFLQWLRHHPDILGRDEPPQCRGPEPRASLSFWELLQGDPWCPDPRSLPLDPPTENALEAPVPSWLPNSWQSQTWAQLVARGESPNNRLYWGLYILLLVAQAIIAAFIVFAMIKIGQLFRTLIREKLLLEAMGKSCN

SEQ ID NO: 34 - protein, 551 aa, Mus musculus
QPFPCPKTCKCVVRDAAQCSGGSVAHIAELGLPTNLTHILLFRMDQGILRNHSFSGMTVLQRLMLSDSHISAIDPGTFNDLVKLKTLRLTRNKISRLPRAILDKMVLLEQLFLDHNALRDLDQNLFQQLRNLQELGLNQNQLSFLPANLFSSLRELKLLDLSRNNLTHLPKGLLGAQVKLEKLLLYSNQLTSVDSGLLSNLGALTELRLERNHLRSVAPGAFDRLGNLSSLTLSGNLLESLPPALFLHVSSVSRLTLFENPLEELPDVLFGEMAGLRELWLNGTHLSTLPAAAFRNLSGLQTLGLTRNPRLSALPRGVFQGLRELRVLALHTNALAELRDDALRGLGHLRQVSLRHNRLRALPRTLFRNLSSLESVQLEHNQLETLPGDVFAALPQLTQVLLGHNPWLCDCGLWPFLQWLRHHPDILGRDEPPQCRGPEPRASLSFWELLQGDPWCPDPRSLPLDPPTENALEAPVPSWLPNSWQSQTWAQLVARGESPNNRLYWGLYILLLVAQAIIAAFIVFAMIKIGQLFRTLIREKLLLEAMGKSCN

SEQ ID NO: 35 - DNA, 15 bp, unidentified
  source 1..15: mol_type = other DNA
ggttggtgtggttgg

SEQ ID NO: 36 - DNA, 29 bp, unidentified
  source 1..29: mol_type = other DNA
agtccgtggtagggcaggttggggtgact

SEQ ID NO: 37 - DNA, 29 bp, unidentified
  source 1..29: mol_type = other DNA
agtccgtaataaagcaggttaaaatgact

SEQ ID NO: 38 - protein, 20 aa, unidentified
MVSAIVLYVLLAAAAHSAFA